(19) United States
(12) Patent Application Publication
(10) Pub. No.: US 2021/0330245 A1
(43) Pub. Date: Oct. 28, 2021

(54) TELEDERMATOLOGY SYSTEM AND METHODS

(71) Applicant: DermTech, Inc., La Jolla, CA (US)

(72) Inventors: John Daniel DOBAK, III, La Jolla, CA (US); Burkhard JANSEN, La Jolla, CA (US); Claudia IBARRA, Carlsbad, CA (US); James ROCK, La Jolla, CA (US); Zuxu YAO, San Diego, CA (US); Sarah DION, San Diego, CA (US)

(21) Appl. No.: 17/236,919

(22) Filed: Apr. 21, 2021

Related U.S. Application Data
(60) Provisional application No. 63/014,057, filed on Apr. 22, 2020.
(60) Provisional application No. 63/106,806, filed on Oct. 28, 2020.

(51) Int. Cl. A61B 5/00; G16H 10/20; G16H 50/30; G16H 80/00

(52) U.S. Cl. CPC A61B 5/444; A61B 5/0077; A61B 5/0004; G16H 80/00; A61B 5/744; G16H 10/20; G16H 50/30; A61B 5/0022

(57) ABSTRACT

Provided herein are systems, platforms, and methods for conducting remote dermatology evaluation and sampling.

### CROSS-REFERENCE TO RELATED APPLICATIONS

This application claims the benefit of U.S. Provisional Application No. 63/014,057 filed Apr. 22, 2020, and U.S. Provisional Application No. 63/106,806 filed Oct. 28, 2020, which applications are incorporated herein by reference for all purposes in their entirety.

### SUMMARY

Provided herein are novel systems and methods for utilizing software (e.g., smart phone app) to assist in the evaluation of and to initiate distribution of non-invasive tissue sampling kits for a patient. A variety of skin conditions can be evaluated and tested effectively using the telehealth solution disclosed herein, including but not limited to melanoma, non-melanoma skin cancer, inflammatory skin conditions, and the like. In one example, non-invasive melanoma skin cancer detection is possible with a pigmented lesion assay (PLA) genomic test that accurately assesses a variety of skin lesions, such as suspicious moles.

Previously, patients with suspicious skin conditions could only receive direct physical examinations through a visit with a clinician/licensed provider, such as a doctor, physician assistant or nurse practitioner. Many patients are unable to easily visit a doctor due to remote locale, physical condition, or even governmental restrictions, such self-isolation requirements. Timely visits to doctors may be critical with certain skin conditions, such as melanoma, where skin cancer is aggressive, and treatment should not be delayed. Additionally, health care costs and timely diagnoses can be difficult for many patients due to limited funds, health care insurance, or even time. Current telemedicine solutions cannot biopsy skin samples remotely and the scope of current telehealth and telemedicine solutions is therefore limited. In-person solutions may be impossible, too late, or may even risk the doctor's exposure in times of a pandemic.

The systems and method described herein provide patients with a computer-implemented solution that allows for easy evaluation of skin conditions and determination of whether a skin collection kit should be used for non-invasive skin collection thereof. Unlike prior teledermatological methods, in some embodiments herein, evaluation and skin sampling are done completely remote, wherein images of skin conditions are captured outside of physician's office. In some examples, a mobile application may be provided that documents and captures images of skin conditions, such as suspicious moles and further facilitates evaluation and determination of whether a genomic test sample collector should be sent to the patient to self-sample at home to enable the assessment of molecular risk factors or prognostic factors. The solution may further enable clinicians to communicate patient information, interact with patients to offer quality care, and to facilitate reporting, shipping and billing. The non-invasive PLA, as an example, is well-suited for use in a telehealth environment because patient care is not delayed, clinician-supervised remote collection avoids unnecessary exposure to patients and physician office staff and PLA test results may guide essential office visit decisions. As a result, the systems and methods allow for skin conditions (e.g., moles) to be evaluated non-invasively for molecular risk factors at performance levels that exceed the current care paradigm of visual inspection, surgical biopsy, and histopathologic assessment that may not be available during pandemic and other crises when many offices may be closed and resources need to be conserved. Further beyond times of crisis, both clinicians and their patients may appreciate the efficient, cost-effective and highly accurate method of evaluating skin conditions.

Provided herein are embodiments of a system for assessing a location on skin of an individual comprising: a first device comprising at least one processor and instructions executable by the at least one processor to provide a first application configured to perform operations comprising: accessing a camera to capture at least one photo of the individual's skin; and submitting a request for a virtual visit for a skin condition of the individual; and a second device comprising at least one processor and instructions executable by the at least one processor to provide a second application configured to perform operations comprising: receiving a notification that the virtual visit is completed by the individual; providing access to an interface for reviewing a record of the virtual visit; providing access to an interface for identifying at least one location on the individual's skin that requires further assessment; and submitting a request to send a non-invasive skin tissue sample kit to the individual. In some embodiments, the second application is configured to perform receiving a report of the non-invasive skin tissue sample. In further embodiments, the report comprises one or more genetic or epigenetic risk factors for the individual. In some embodiments, the second application is configured to perform providing an interface for interpreting the report and communicating results of the skin tissue sample to the individual. In various embodiments, the skin condition comprises melanoma and non-melanoma skin cancer, psoriasis, atopic dermatitis, or sun damage. In some embodiments, the virtual visit is asynchronous. In some embodiments, the second application is configured to perform initiating the virtual visit by sending link to the individuals. In some embodiments, the first application is implemented as a mobile application. In some embodiments, the second application is implemented as a web application. In other embodiments, the second application is implemented as a mobile application. In some embodiments, the record of the virtual visit comprises a location of the skin condition, one or more images of the skin condition, and responses to a survey about the skin condition. In some embodiments, the one or more images comprise at least one photo, at least one video, or a combination thereof. In some embodiments, the non-invasive skin tissue sample kit comprises an adhesive sampling material. In some embodiments, the second application is configured to perform providing an interface for initiating a follow-up visit for the individual. In some embodiments, the second application is configured to perform providing an interface for prescribing a treatment to the individual based at least in part on results of the skin tissue sample, the record of the virtual visit, or a combination thereof.

Provided herein are embodiments of a method for assessing a location on skin comprising: a first individual accessing an imaging implement to capture at least one image of the first individual's skin; the first individual submitting a request for a virtual visit for a skin condition of the first individual; a second individual receiving a notification that the virtual visit is completed by the first individual; the second individual accessing a portal to review a record of the virtual visit; the second individual identifying at least one location on the first individual's skin that requires further assessment; and the second person submitting a request to send a non-invasive skin tissue sample kit to the first individual. In some embodiments, the method further comprises the second individual receiving a report of the non-invasive skin tissue sample. In some embodiments, the report comprises one or more genetic or epigenetic risk factors for the first individual. In some embodiments, the method further comprises the second individual interpreting the report and communicating results of the skin tissue sample to the first individual. In various embodiments, the skin condition comprises melanoma and non-melanoma skin cancer, psoriasis, atopic dermatitis, or sun damage. In some embodiments, the virtual visit is asynchronous. In some embodiments, the method further comprises the second individual initiating the virtual visit by sending link for the first individual to access a mobile application. In some embodiments, the portal is implemented as a web application. In other embodiments, the portal is implemented as a mobile application. In some embodiments, the record of the virtual visit comprises a location of the skin condition, one or more images of the skin condition, and responses to a survey about the skin condition. In some embodiments, the one or more images comprise at least one photo, at least one video, or a combination thereof. In some embodiments, the non-invasive skin tissue sample kit comprises an adhesive sampling material. In some embodiments, the method further comprises the second individual initiating a follow-up visit for the first individual. In some embodiments, the method further comprises the second individual administering a treatment to the first individual based at least in part on results of the skin tissue sample, the record of the virtual visit, or a combination thereof.

Provided herein are embodiments of a teledermatology system comprising a device comprising at least one processor and instructions executable by the at least one processor to provide a teledermatology application configured to perform operations comprising: receiving a request for teledermatology visit for a skin condition; mapping a location of the skin condition; capturing one or more images of the skin condition; providing information about the skin condition; uploading the location, the one or more images, and the information to a back-end system; receiving a notification of provision of a non-invasive skin tissue sample kit; and receiving results of the skin tissue sample. In various embodiments, the skin condition comprises melanoma and non-melanoma skin cancer, psoriasis, atopic dermatitis, or sun damage. In some embodiments, the teledermatology visit is asynchronous. In some embodiments, the teledermatology application is configured to perform receiving a link from a healthcare provider to initiate the teledermatology visit. In some embodiments, mapping the location of the skin condition comprises mapping the location onto an avatar. In some embodiments, capturing the one or more images of the skin condition comprises adjusting illumination, focus, or framing. In some embodiments, the one or more images comprise a plurality of images and the images are captured substantially simultaneously using more than one camera of the device. In some embodiments, the one or more images comprise a photo, a video, or a combination thereof. In some embodiments, providing information about the skin condition comprises responding to a survey by selecting responses from pre-determined options. In some embodiments, the non-invasive skin tissue sample kit comprises an adhesive sampling material. In some embodiments, the results of the skin tissue sample comprise one or more genetic or epigenetic risk factors. In some embodiments, the teledermatology application is configured to perform receiving a request for a follow-up visit. In some embodiments, the teledermatology application is configured to perform receiving notification of a treatment in response to the results of the skin tissue sample, the teledermatology visit, or a combination thereof.

Provided herein are embodiments of a teledermatology method comprising: request a teledermatology visit for a skin condition of a patient; receive a notification that the teledermatology visit is completed by the patient; access a healthcare provider portal to review a record of the teledermatology visit; determine, based on the review, to administer a non-invasive skin tissue sample of the location of the skin condition and initiate sending a non-invasive skin tissue sample kit to the patient; receive a report of the non-invasive skin tissue sample; and interpret the report and communicate results of the skin tissue sample to the patient.

In some embodiments, the skin condition comprises melanoma and non-melanoma skin cancer, psoriasis, atopic dermatitis, or sun damage. In some embodiments, the skin condition comprises melanoma and non-melanoma skin cancer, psoriasis, atopic dermatitis, or sun damage the teledermatology visit is asynchronous. In some embodiments, the method further comprises initiating the teledermatology visit by sending a link for the patient to access a mobile teledermatology application.

In some embodiments, the healthcare provider portal is implemented as a web application. In some embodiments, the healthcare provider portal is implemented as a mobile application. In some embodiments, the record of the teledermatology visit comprises a location of the skin condition, one or more images of the skin condition, and responses to a survey about the skin condition. In some embodiments, the one or more images comprise a photo, a video, or a combination thereof.

In some embodiments, the non-invasive skin tissue sample kit comprises an adhesive sampling material. In some embodiments, the report comprises one or more genetic or epigenetic risk factors for the patient. In some embodiments, the method further comprises initiating a follow-up visit for the patient. In some embodiments, the method further comprises administering a treatment to the patient based at least in part on the results of the skin tissue sample, the record of the teledermatology visit, or a combination thereof.

Provided herein are embodiments of a teledermatology method comprising: receive a request for teledermatology visit for a skin condition; access a mobile teledermatology app via a mobile device; use the mobile teledermatology app to map a location of the skin condition; use the mobile teledermatology app to capture one or more images of the skin condition; use the mobile teledermatology app to provide information about the skin condition; use the mobile teledermatology app to upload the location, the images, and the information to a back-end system; receive a non-invasive skin tissue sample kit and perform the sampling; receive results of the skin tissue sample.

In some embodiments, the skin condition comprises melanoma and non-melanoma skin cancer, psoriasis, atopic dermatitis, or sun damage. In some embodiments, the teledermatology visit is asynchronous. In some embodiments, the method further comprises receiving a link from a healthcare provider to access a mobile teledermatology application. In some embodiments, mapping the location of the skin condition comprises mapping the location onto an avatar.

In some embodiments, capturing the one or more images of the skin condition comprises adjusting illumination, focus, or framing. In some embodiments, the one or more images comprises a plurality of images and the images are captured simultaneously using more than one camera of the mobile device. In some embodiments, the one or more images comprise a photo, a video, or a combination thereof.

In some embodiments, providing information about the skin condition comprises responding to a survey by selecting responses from pre-determined options. In some embodiments, the non-invasive skin tissue sample kit comprises an adhesive sampling material. In some embodiments, the results comprise one or more genetic or epigenetic risk factors.

In some embodiments, the method further comprises receiving a request for a follow-up visit. In some embodiments, the method further comprises receiving a treatment in response to the results of the skin tissue sample, the teledermatology visit, or a combination thereof.

Provided herein are embodiments of a cloud-based back-end system for teledermatology configured to perform operations comprising: receiving a request generated at a healthcare provider portal for a teledermatology visit for a skin condition of a patient; transmitting the request for the teledermatology visit to a patient at a patient mobile teledermatology application; receiving a record of the teledermatology visit performed at the patient mobile teledermatology application, the record comprising: a location of the skin condition, one or more images of the skin condition, and responses to a survey about the skin condition; transmitting a notification to the healthcare provider portal that the teledermatology visit is completed by the patient; providing the record of the teledermatology visit at the healthcare provider portal; receiving a request for a non-invasive skin tissue sample of the location of the skin condition and initiating sending a non-invasive skin tissue sample kit to the patient; receiving a report of the non-invasive skin tissue sample; and communicating the report of the skin tissue sample to the patient mobile teledermatology application and the healthcare provider portal.

Provided herein are embodiments of a teledermatology method comprising: receive, at a back-end system, a request generated at a healthcare provider portal for a teledermatology visit for a skin condition of a patient; transmit, by the back-end system, the request for the teledermatology visit to a patient at a patient mobile teledermatology application; receive, at the back-end system, a record of the teledermatology visit performed at the patient mobile teledermatology application, the record comprising: a location of the skin condition, one or more images of the skin condition, and responses to a survey about the skin condition; transmit, by the back-end system, a notification to the healthcare provider portal that the teledermatology visit is completed by the patient; provide, by the back-end system, the record of the teledermatology visit at the healthcare provider portal; receive, at the back-end system, a request for a non-invasive skin tissue sample of the location of the skin condition and initiate sending a non-invasive skin tissue sample kit to the patient; receive, at the back-end system, a report of the non-invasive skin tissue sample; and communicate, by the back-end portal, the report of the skin tissue sample to the patient mobile teledermatology application and the healthcare provider portal.

Provided herein in some embodiments are methods of remote skin evaluation and tissue sampling comprising: conducting a remote evaluation of a skin condition of a patient, wherein the remote evaluation is conducted without direct physical contact of the patient by a physician; and performing a subsequent procedure in response to the remote evaluation. In some embodiments, the subsequent procedure comprises ordering a non-invasive tissue collection kit, wherein the non-invasive tissue collection kit enables remote tissue sample collection without direct physical contact of the patient by a physician. In some embodiments, the methods further comprise reporting results of a tissue sample test. In some embodiments, the methods further comprise a step of performing the tissue sample test prior to reporting the results of the tissue sample test. In some embodiments, the subsequent procedure comprises capturing images of the skin condition at later date. In some embodiments, the subsequent procedure comprises treating the skin condition.

Provided herein, in some embodiments, are methods for providing remote dermatological skin evaluation and tissue sampling to a patient without necessary direct physical contact, including: initiating the remote dermatological skin evaluation; evaluating a skin condition; ordering a non-invasive skin collection kit; and reporting results of a skin tissue sample test. In some embodiments, initiating the remote dermatological skin evaluation is conducted by a physician, a licensed provider, a professional, a patient, a partner, a caretaker, an insurance company, an employer, a web search, a web advertisement, or a combination thereof. In some embodiments, the step of evaluating the skin condition is responsive to the step of initiating the remote dermatological skin evaluation. In some embodiments, the step of ordering the non-invasive skin collection kit is responsive to the step of evaluating the skin condition. In some embodiments, the method further comprises a step of validating the skin condition, wherein the step of ordering the non-invasive skin collection kit is responsive to the step of validating the skin condition. In some embodiments, the method further comprises a step of approving the ordering of the non-invasive skin collection kit. In some embodiments, the step of ordering the non-invasive skin collection kit is carried out by a first entity, and wherein the step of approving the ordering is carried out by a second entity, wherein the first entity and the second entity are different. In some embodiments, the method further comprises a step of testing the skin sample prior to the step of reporting results of the skin sample test.

Provided herein are embodiments of a computer implemented remote dermatological skin evaluation software system comprising: a patient-facing digital processing device comprising at least one processor, an operating system configured to perform executable instructions, a memory, and a computer program including instructions executable by the digital processing device to create a patient-facing skin evaluation application comprising: an image collection feature; and a physician-facing digital processing device comprising at least one processor, an operating system configured to perform executable instructions, a memory, and a computer program including instructions executable by the digital processing device to create a physician-facing skin evaluation application comprising: an imaging viewing feature, and an ordering feature for ordering a non-invasive skin collection kit. In some embodiments, the patient-facing skin evaluation application further comprises a questionnaire feature. In some embodiments, the downloadable the patient-facing skin evaluation application further comprises a skin tracking feature. In some embodiments, the patient-facing skin evaluation application further comprises a result viewing feature to display results of a skin tissue sample test. In some embodiments, the physician-facing skin evaluation application further comprises a result viewing feature to display results of a skin tissue sample test. In some embodiments, the patient-facing digital processing device further comprises at least one optical device for recording images. In some embodiments, the physician-facing digital processing device is configured to remotely capture images using the at least one optical device of the patient-facing digital processing device.

Provided herein are embodiments of a cloud-based system for providing remote dermatological skin evaluation and tissue sampling to a patient without necessary direct physical contact, comprising: a module for initiating the remote dermatological skin evaluation; a module for evaluating a skin condition; a module for ordering a non-invasive skin collection kit; and a module for reporting results of the testing of the skin tissue sample. In some embodiments, the system further comprises a module comprising a questionnaire to be completed by the patient. In some embodiments, the module for ordering the non-invasive skin collection kit is configured to receive instructions for approval from a third-party networked device comprising a processor configured to perform executable instructions. In some embodiments, the system further comprises a module to map a location of the skin condition. In some embodiments, the location of the skin condition is mapped onto an avatar representative of a body of the patient.

Provided herein are embodiments of a system comprising: a first networked device comprising a processor configured to perform executable instructions, the first device comprising a least one camera configured to capture one or more images; a second networked device comprising a processor configured to perform executable instructions, wherein the first and second networked devices each comprise a program including instructions executable by the device to create a software application comprising: a module for remote monitoring by a telemedical care provider; and a module for applying a diagnostic analysis of a health condition. In some embodiments, the software application further comprises a module for telecommunications between the first or second device. In some embodiments, the second device is operated by the telemedical care provider. In some embodiments, the software application further comprises a module to allow the telemedical care provider to remotely operate the first device. In some embodiments, the module allowing the telemedical care provider to remotely operate the first device allows the telemedical care provider to remotely adjust zoom, focus, lighting, and other parameters of the at least one camera of the first device and/or to remotely capture images using the at least one camera of the first device. In some embodiments, the system further comprises a module for transmitting the diagnostic analysis from the second device to the first device.

In some embodiments, the health condition is a skin condition. In some embodiments, the skin condition comprises inflammatory skin conditions, skin cancer, skin conditions caused by pathogens, immune mediated skin conditions, drug reactions, medical conditions, wound healing, radiation damage, ultraviolet light damage, or a combination thereof. In some embodiments, the pathogens comprise bacteria, viruses, fungi, protozoa, parasites, or a combination thereof. In some embodiments, the skin cancer comprises melanoma, carcinoma, or a combination thereof. In some embodiments, the inflammatory skin conditions comprise Psoriasis, Psoriatic Arthritis, Atopic Dermatitis, Atopic Asthma, Vitiligo, Lupus, Cutaneous T-cell lymphoma, Alopecia, Alopecia Areata, or a combination thereof.

In some embodiments, the system further comprises a module to provide a questionnaire to a patient on the first device. In some embodiments, the system further comprises a module to allow the telemedical care provider to order one or more diagnostic tools. In some embodiments, the one or more diagnostic tools comprise a diagnostic light, a magnifying lens, or a combination thereof. In some embodiments, the magnifying lens is configured to attach to the at least one of camera of the first device. In some embodiments, the diagnostic light emits light in the ultraviolet spectrum. In some embodiments, the system further comprises an ultraviolet camera to perform a spectral analysis of a skin area when the ultraviolet light is incident on said skin area. In some embodiments, the diagnostic light emits light in the infrared spectrum. In some embodiments, the system further comprises an infrared camera to perform a spectral analysis of a skin area when the infrared light is incident on said skin area.

In some embodiments, the system further comprises a tracking module configured to track one or more skin abnormalities. In some embodiments, the tracking module compiles a plurality of images captured by the camera of the first device taken over a period of time. In some embodiments, the tracking module maps the location of the one or more skin abnormalities on a body of a patient or user. In some embodiments, the tracking module is configured to display the location of the one or more skin abnormalities on an avatar representing the body of a patient or user.

In some embodiments, the system further comprises a module to allow the telemedical care provider to order a skin test. In some embodiments, further comprises a module to provide an alert when results from the skin test are available. In some embodiments, the system further comprises a module to display the results from the tissue skin test on the first device, the second device, or both.

In some embodiments, the skin test comprises a tissue sampling kit. In some embodiments, the tissue sampling kit comprises a microneedle. In some embodiments, a skin sample collected by the microneedle comprises from about 50 microgram to about 1 gram of cellular material. In some embodiments, the skin sample collected by the microneedle comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of RNA material.

In some embodiments, the tissue sampling kit comprises a skin brush. In some embodiments, the skin sample collected by the skin brush comprises from about 50 microgram to about 1 gram of cellular material. In some embodiments, the skin sample collected by the brush comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of RNA material.

In some embodiments, the tissue sampling kit comprises a scraper. In some embodiments, a skin sample collected by the scraper comprises from about 50 microgram to about 1 gram of cellular material. In some embodiments, the skin sample collected by the scraper comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of RNA material.

In some embodiments, the tissue sampling kit comprises an adhesive patch skin sample collector system. In some embodiments, the adhesive patch skin sample collector system comprises: a peelable release panel comprising 2 to 12 adhesive tapes; a placement area panel comprising a removable liner and 2 to 12 regions wherein each of the 2 to 12 regions is designated for the placement of a used adhesive tape; and a clear panel, wherein the adhesive tape is configured for application to a skin surface so that an effective amount of a skin sample adheres to an adhesive matrix of the adhesive tape. In some embodiments, the effective amount of the skin sample comprises from about 50 microgram to about 1 gram of cellular material. In some embodiments, the cellular material is a nucleic acid. In some embodiments, the cellular material is RNA or DNA. In some embodiments, the adhesive patch skin sample collector system is labeled with a unique barcode that is assigned to a patient sample. In some embodiments, the peelable release panel is configured to hold between 4 to 10 adhesive tapes, between 6 to 10 adhesive tapes, between 6 to 8 adhesive tapes, or between 4 to 8 adhesive tapes. In some embodiments, the placement area panel is configured to hold between 4 to 10 adhesive tapes, between 6 to 10 adhesive tapes, between 6 to 8 adhesive tapes, or between 4 to 8 adhesive tapes. In some embodiments, the peelable release panel is configured to hold 4 adhesive tapes and the placement area panel is configured to hold 4 adhesive tapes. In some embodiments, the peelable release panel is configured to hold 8 adhesive tapes and the placement area panel is configured to hold 8 adhesive tapes. In some embodiments, the effective amount of the skin sample comprises between about 50 microgram to about 500 microgram, between about 100 microgram to about 450 microgram, between about 100 microgram to about 350 microgram, between about 100 microgram to about 300 microgram, between about 120 microgram to about 250 microgram, or between about 150 microgram to about 200 microgram of RNA material.

In some embodiments, the adhesive tape comprises a first central collection area having a skin-facing surface comprising the adhesive matrix and a second area extending from the periphery of the first collection area creating a tab. In some embodiments, the first central collection area and the second area are comprised of different materials. In some embodiments, the first central collection area is comprised of a polyurethane carrier film. In some embodiments, the first central collection area has a second surface for demarcation of a zone around a skin lesion.

In some embodiments, the diagnostic analysis comprises a visual inspection by the telemedical care provider. In some embodiments, the visual inspection comprises an analysis of the one or more images captured by the at least one camera of the first device. In some embodiments, wherein the visual inspection comprises an analysis a video captured by the at least one camera of the first device.

Provided herein are embodiments of a method for diagnosing a skin condition of a patient comprising: capturing one or more images of one or more skin areas, the one or more skin areas being affected by a skin condition; sending the one or more images to a device of a telemedical care provider; evaluating the one or more images; and diagnosing the skin condition the patient.

In some embodiments, the method further comprises a step of performing a spectral analysis on the one or more skin areas prior to diagnosing the skin condition. In some embodiments, the step of performing a spectral analysis comprises emitting a light having a frequency of interest incident on the one or more skin areas. In some embodiments, the frequency of interest is an ultraviolent frequency, an infrared frequency, or a combination thereof.

In some embodiments, the method further comprises receiving reflected light from the one or more skin areas with a camera configured to capture the reflected light at the frequency of interest. In some embodiments, the method further comprises a step of tracking the one or more skin areas.

In some embodiments, the step of tracking the one or more skin areas comprises tracking sizes of the skin condition affecting each of the one or more skin areas. In some embodiments, tracking sizes of the skin condition comprises capturing one or more images of the skin condition over a duration of time. In some embodiments, the step of evaluating the one or more images comprises evaluating a change in color of the skin condition over the duration of time. In some embodiments, the step of evaluating the one or more images comprises evaluating a change in size of the skin condition over the duration of time. In some embodiments, the step of tracking the one or more skin areas comprises tracking one or more locations of the skin condition affecting each of the one or more skin areas.

In some embodiments, the method further comprises mapping the one or more locations of the skin conditions on the patient. In some embodiments, the one or more locations of the skin conditions on the patient are mapped onto an avatar representing a body of the patient.

In some embodiments, the method further comprises a step of ordering one or more skin tests prior to the step of diagnosing the skin condition. In some embodiments, the method further comprises steps of sending a tissue sampling kit to the patient; and collecting one or more tissue samples from the one or more skin areas for the one or more skin tests. In some embodiments, the patient conducts the step of using the tissue sampling kit to collect one or more tissue samples of the one or more skin areas. In some embodiments, the tissue sampling kit comprises an adhesive patch skin sample collector method.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a non-limiting example of a computing device; in this case, a device with one or more processors, memory, storage, and a network interface;

FIG. 2 shows a non-limiting example of a web/mobile application provision system; in this case, a system providing browser-based and/or native mobile user interfaces;

FIG. 3 shows a non-limiting example of a cloud-based web/mobile application provision system; in this case, a system comprising an elastically load balanced, auto-scaling web server and application server resources as well synchronously replicated databases;

FIG. 4 depicts an exemplary graphical user interface for presenting captured images of a skin area;

FIGS. 5A and 5B depict a workflow of a method for remote teledermatology;

FIGS. 6A-6AA depict an exemplary graphical user interfaces comprising a patient user experience (GUI/UX) for collecting images and information corresponding to a skin area to be submitted for evaluation;

FIGS. 7-14 depict an exemplary tissue sample collection kit and a method of use thereof;

FIG. 15 depicts a non-limiting example of a platform to connect healthcare professionals with patients in need of care, according to some embodiments described herein; and

FIG. 16 depicts an exemplary method for conducting a remote dermatology visit.

### DETAILED DESCRIPTION

Provided herein are systems and methods for a remote dermatology evaluation. In some embodiments, after an evaluation is completed a sample collection kit is sent to the location of a patient in need thereof. In some embodiments, the sample collection kit is a non-invasive sample collection kit which may be self-administered. After collection of one or more skin or tissue samples, the collection kit, along with the collected samples, may be sent to a lab for testing. The results of the test may be communicated by a medical professional to the patient. In some embodiments, results are communicated via a software application downloaded on to a user device accessible by the patient.

### I. Remote Dermatological Skin Evaluation

In some embodiments, a method for providing remote dermatological skin evaluation and tissue sampling to a patient without direct physical contact is provided herein. In some embodiments, the method comprises an initiation of skin evaluation. In some embodiments, the method comprises a process for determining or validating if a skin evaluation is needed for a patient or user.

In some embodiments, initiation of a skin evaluation is made by a physician. In some embodiments, the physician specializes in dermatology or is a dermatologist. In some embodiments, initiation of a skin evaluation is made by a licensed provider. The licensed provider may specialize in dermatology or be a licensed dermatologist. In some embodiments, initiation of a skin evaluation is made by another healthcare professional. The healthcare professional may be a general physician, nurse practitioner, or dermatology specialist. In some embodiments, methods of initiation of a skin evaluation, as described herein, are performed through modules or features of a software application, such as a mobile application or a web-based application.

In some embodiments, a skin care evaluation process is initiated during a communication between a patient or user and a telemedical operator, a physician, a licensed provider, healthcare professional, or another profession. The communication may be a phone call, a text message, a video call, email, automated phone call (including a call from a robot calling system) scheduled visit with a profession, or a communication via a software application. The initiation may begin with a request, questionnaire, or other communication from a patient or user expressing concern regarding a skin condition or symptom related to a skin condition. In some embodiments, initiation may begin with a telemedical operator, a physician, a licensed provider, healthcare professional, or another profession gathering information from a patient or user and determining that a skin evaluation may be necessary after evaluating the information received from the patient or user.

In some embodiments, a skin evaluation initiated after assessment of a patient's electronic medical record (EMR). The assessment of may be of a previous EMR, a current EMR, or an EMR being processed. In some embodiments, the initiation of a skin evaluation may occur after an assessment of an image captured in a clinical setting. In some embodiments, the captured image may include an image as part of an electronic medical record.

In some embodiments, a system for remote skin evaluation includes a physician-facing software application. The system for remote skin evaluation may include be implemented on various computer systems, as described herein. The physician-facing software may include an application directed to receive input from a physician, licensed, professional, telemedical healthcare provider, or other professional. The physician-facing software may be utilized to send their patient(s) an email with a link to download a mobile application and schedule a virtual visit to capture images of one or more skin conditions or suspected skin condition with the patient. In some embodiments, the patient receives an email providing information about the application. In some embodiments, the patient may click on a link provided in the email to download the software application onto their iOS or Android phone. The application (app) may be considered a patient-facing software application.

In some embodiments, a patient registers or confirms their pre-loaded patient information, such as name, age, mobile phone number, address, physician Information, electronic medical record, or other personal information during initiation of a skin evaluation via the software application. In some embodiments, the registration feature is provided in a software module of the application. In a cloud-based system, the registration module may be provided via a web based application. The patient information may be confirmed or registered with an electronic medical record. In some embodiments, a patient registers or confirms their pre-loaded patient information via a phone call. The phone call may be a 3-way phone call. The 3-way phone call may include the patient, a medical technician, and a physician, licensed, professional, telemedical healthcare provider, or other professional.

In some embodiments, a skin evaluation may be initiated by a person other than a licensed clinician. A skin evaluation may be initiated by patient, partner, caretaker. In some embodiments, a patient initiates a skin evaluation upon discovery of a suspicious looking lesion. In some embodiments, an initiation feature or module is integrated with a patient-management software or patient-facing application. In some embodiments, one or more caretakers are granted access to initiate skin evaluations for group of clients or patients. For example, in a skilled nursing facility, one or more caretakers could initiate skin care evaluations for residents or patients of the skilled nursing facility.

In some embodiments, an insurance company may initiate a skin evaluation. The skin evaluation may be used during assessment for preexisting conditions for an applicant for health, life, or other type of insurance requiring a health assessment. In some embodiments, an insurance company may initiate a skin evaluation, as described herein, as part of a regular risk analysis for patients or applicants. In some embodiments, the insurance company may initiate a skin evaluation for patients having a history of skin conditions. In some embodiments, the insurance company may initiate a skin evaluation for patients above a predetermined age.

In some embodiments, initiation of a skin evaluation may be automated. Automated initiation may be passed on information provided in an EMR, risk assessment, and/or other medical history. In some embodiments, initiation of a skin evaluation by an insurance company may be automated.

In some embodiments, a skin evaluation may be initiated by an employer, wellness program, wellness fair, or in a concierge medicine setting/programs. In some example embodiments, a patient enters a skin spa and in the registration an image is captured using facial scanning technology. A profile of the patient may be stored, processed, and used to initiate a skin evaluation process. The patient may be contacted to initiate the skin evaluation or sent a questionnaire to determine if a skin evaluation is recommended.

In some embodiments, a skin evaluation may be initiated by a website, posting, electronic advertisement, text recognition (via input received in a search engine), or other means which may utilize a user's electronic history. In some examples, a patient may conduct a web search using keywords such as skin cancer, melanoma, psoriasis, and other skin conditions that may be tested by non-invasive tissue sampling described herein. The web search may then be utilized to produce electronic banners or advertisements to direct the user to initiate a skin evaluation. In some examples, a user or patient will click on a presented electronic advertisement or banner and is then taken to a registration/initiation page.

In some embodiments, a skin evaluation may be initiated in response to ongoing skin tracking (e.g., MoleMapper™), UV scores (e.g., such as store in HealthKit), in response to an artificial intelligence-enabled lesion analysis, or image recognition algorithm. In some embodiments, a skin evaluation may be initiated by a software application that assist in patient-facing dermatological evaluation, e.g., SkinVision, UMSkinCheck, Dermatology A to Z, CureSkin, Skin Image Search by First Derm, and the like.

In some embodiments, initiation of a skin evaluation occurs after data collected from a clinical trial, marketing study, marketing analysis, layman's assessment of use is evaluated and risk of a skin condition is determined.

In some embodiments, a determination of the necessity of a skin evaluation is made by a physician. In some embodiments, the physician specializes in dermatology or is a dermatologist. In some embodiments, a determination of the necessity of a skin evaluation is made by a licensed provider. The licensed provider may specialize in dermatology or be a licensed dermatologist. In some embodiments, a determination of the necessity of a skin evaluation is made by another healthcare professional. The healthcare professional may be a general physician, nurse practitioner, or dermatology specialist.

Provided herein are methods and systems for evaluation of a skin condition. In some embodiments, methods for evaluation of a skin condition are performed via features or modules of software, such as a mobile application or a web-based application. In some embodiments, a skin evaluation is responsive to an initiation as described above. In some embodiments, a validation step occurs wherein initiation of a skin evaluation is reviewed by a telemedical operator, a physician, a licensed provider, healthcare professional, or another profession. The validation may be provided in the context of a cloud-connected evaluation application or software application loaded onto a connected device.

In some embodiments, the skin evaluation utilizes image collection software, such as video or photographic capturing hardware and software. In some embodiments, an image capture feature captures characteristics of mole through software-based image capture and analysis.

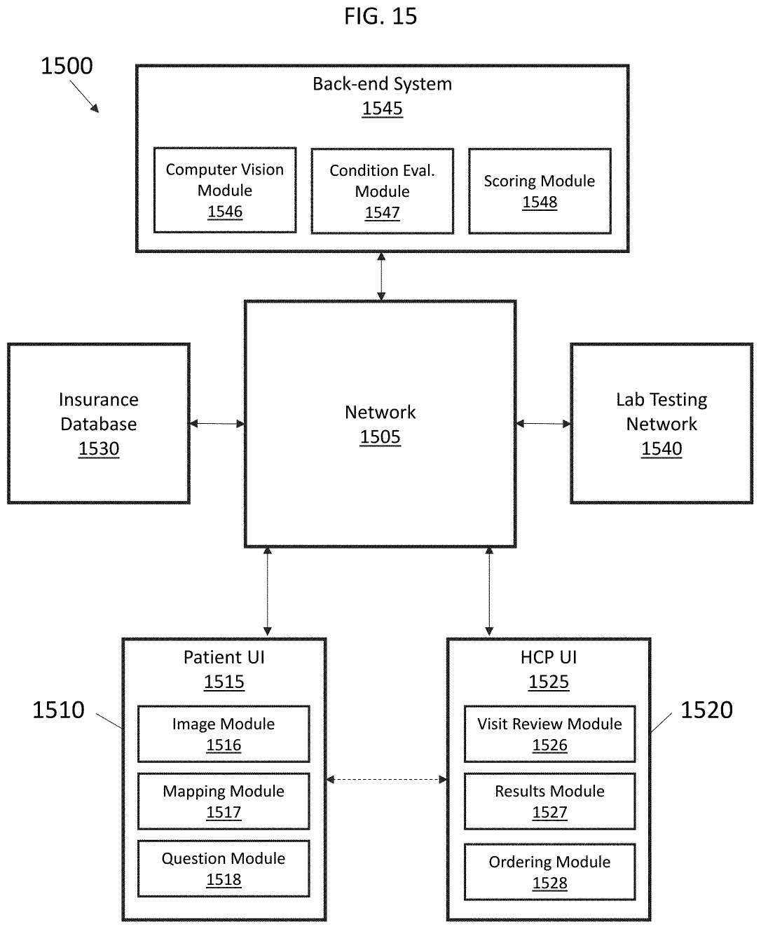

In some embodiments, a software based analysis utilizes provided patient information. In some embodiments, the information is provided by a patient questionnaire (see, e.g., FIG. 15, 1518). In some embodiments, the information is provided by a telemedical operator, a physician, a licensed provider, healthcare professional, partner, or caretaker initiating the skin evaluation. In some embodiments, the information includes skin condition information. In some embodiments, the skin condition information includes the size, color, borders, or other characteristics of a mole, lesion, or affected skin area. In some embodiments, color may include an assessment of the redness of the affected skin area. Augmenting factors and data can also include family and patient history, patient age, gender, underlying and additional health conditions. In some embodiments, computer-implemented algorithms are used to assess the effect of augmenting factors and data in relation to a skin condition.

In some embodiments, an image collection software, software application, feature, or module includes instructions on how to capture a usable image. In some embodiments, instructions for capturing a proper image are displayed on a screen of a user device utilizing the patient-facing software.

In some embodiments, a patient will utilize a camera of a user device to capture an image of a mole, lesion, or affected skin area. In some embodiments, the image capturing software includes a feature or module (see e.g., FIG. 15, 1516) to capture multiple images of the affected skin area simultaneously. In some embodiments, the image capturing software allows simultaneously capture an overview image and cropped close-up image during a single image capturing event.

In some embodiments, one or more cameras and/or one or camera accessories are used to take a regional image and a close-up image of same lesion or area of skin affected by a skin condition. Multiple images may be collected simultaneously with one tap of the screen for convenience and accuracy. In some embodiments, the close-up image utilizes a zoom which could be a 2×, 3×, 4×, or any other suitable zoom range for capturing a close-up image of the lesion. The zoom may be a digital zoom or an optical zoom. In some embodiments, an image capturing system may use multiple lenses together (e.g., on iPhone X, Samsung Galaxy S10, or other mobile devices having multiple lenses and/or cameras). In some embodiments, images are captured. with each of the different cameras simultaneously. The software application or module may show split a screen result with both regional and zoomed views. In some embodiments, the user then selects/delineates lesion on the zoom view (and/or the regional view). The user may then identify the mole or lesion of interest to differentiate from other moles in an image frame to make clear what lesion the clinician (or analysis software) should focus on.

In some embodiments, image capture of affected skin areas includes a spectral analysis of the skin. In some embodiments, light comprised of a specific wavelengths emitted onto the skin an analysis is performed on reflected light captured by an imaging device of the system. In some embodiments, spectral analysis is conducted in a frequency band to include ultraviolet (UV) and/or infrared (IR) wavelengths. Light sources may be polarized. Use of particular frequency bands may enable looking at deeper layers of the skin, compared to use of light in the visible spectrum. The imaging capture system may use special cameras, optical devices, and imaging devices for spectral analysis. In some embodiments, the imaging devices are capable of detecting heat differences. In some embodiments, infrared photography is utilized to differentiate skin lesions with blood flow differences due to inflammation, infection, or malignant change. In some embodiments, spectral analysis utilizes UV and short-wave imaging and photography. UV and short-wave imaging may be used to detect phenotypic changes in pigment and other factors such as aging, skin cancer risk, skin cancer assessment. Filters over the camera or applied to a digital image may be used to highlight aspects of lesional or non-lesional areas of the skin.

In some embodiments, the system may include an additional imaging apparatus to be used in conjunction with a camera and/or light of a mobile device. In some embodiments, the imaging apparatus is removably attached to a mobile device. In some embodiments, the imaging apparatus is distributed to a patient, partner, or caretaker prior to the skin evaluation.

In some embodiments, an image capturing software records information regarding size, color, borders, or other characteristics of a mole, lesion, or affected skin area with analysis of the captured image(s).

In some embodiments, results of the skin evaluation, such as determination of size or color of lesion, enables automated triggering of additional actions, such as scheduling an in-person appointment and/or biopsy. In some embodiments, results of the skin evaluation populate options such as requests for a tissue sampling kit, consultation, or follow-up pictures. In some embodiments, the options are populated based on each captured image of an affected skin area.

In some embodiments, an affected skin area is tracked over a duration of time to determine if further imaging, processing, sampling, or consulting is necessary. In some embodiments, a skin evaluation based on a capture image of an affected area results for a request and instructions to capture follow up images after a specified time period. In some embodiments, the specified time period is about 7 days to about 120 days. In some embodiments, the specified time period is about 7 days to about 14 days, about 7 days to about 30 days, about 7 days to about 60 days, about 7 days to about 90 days, about 7 days to about 120 days, about 14 days to about 30 days, about 14 days to about 60 days, about 14 days to about 90 days, about 14 days to about 120 days, about 30 days to about 60 days, about 30 days to about 90 days, about 30 days to about 120 days, about 60 days to about 90 days, about 60 days to about 120 days, or about 90 days to about 120 days. In some embodiments, the specified time period is about 7 days, about 14 days, about 30 days, about 60 days, about 90 days, or about 120 days, including increments therein. In some embodiments, the specified time period is at least about 7 days, about 14 days, about 30 days, about 60 days, or about 90 days, including increments therein. In some embodiments, the specified time period is at most about 14 days, about 30 days, about 60 days, about 90 days, or about 120 days, including increments therein.

In an embodiment, two or more images captured over a specified period of time are presented. The images may be displayed on a user-facing device, physician-facing device, or provided in another form for assessment by a patient, partner, caretaker, telemedical operator, a physician, licensed provider, or other healthcare professional. The images may be of the same affected region, mole, or skin condition (including non-lesion skin conditions). In some embodiments, the images are displayed side by side in an evaluation module. In some embodiments, the image may include data relating to observed changes in the image. The data may include data captured by an imaging sensor, analyzed by imaging software, input by a user, or a combination thereof. In some embodiments, displayed images include an automated change identifier. An automated change identifier may include an overlaid image. The overlaid image may include representation of a border of a mole, lesion, or affected skin condition.

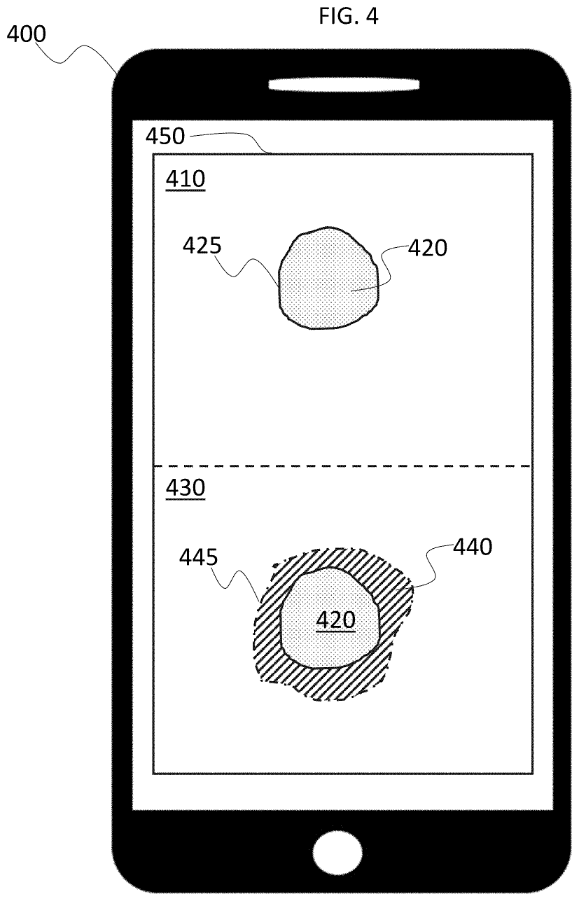

With reference to FIG. 4, an embodiment of an evaluation user interface 450 is depicted as being displayed on a device 400. In some embodiments, a first image 410 of an affected region, mole, or skin condition 420 is displayed. In some embodiments, the image comprises an outline 425 of the skin condition to highlight the border of the skin condition at the time the image is captured.

In some embodiments, the outline 425 is generated by a user input or drawing using an interface of the device 400. In some embodiments, a computer vision algorithm is implemented (see, e.g., at FIG. 15, 1546) to apply the outline 425 to the image of the skin condition. In some embodiments, the computer vison applied algorithm is verified or adjusted via user input. In some embodiments, the application of an outline to an image of a skin condition is completely automated using computer vision techniques.

In some embodiments, a second image 430 of an affected region, mole, or skin condition 440 is displayed. In some embodiments, the second image 430 is of the same affect region, mole, or skin condition captured at a later time after a specified time period has passed since the first image 410 was captured. In some embodiments, the second image comprises an outline 445 of the skin condition to highlight the border of the skin condition.

In some embodiments, a first image of the skin condition 420 is overlaid onto a second image 440 of the skin condition. In some embodiments, an earlier captured image of a skin condition is overlaid on to one or more later captured images of the same skin condition. Overlaying earlier images on top of later images allows for a visual comparison of the changes in size, shape, and color of the skin condition. In some embodiments, images of other lesional areas or moles are captured for comparison to a lesion or mole of interest. In some embodiments, images on non-lesional regions captured for comparison to a lesion or mole of interest.

In some embodiments, computer vision techniques and methods are utilized to assess captured images of lesions or other skin conditions of interest. In some embodiments, computer vision techniques and methods are utilized to identify features of captured images of lesions or other skin conditions of interest. In some embodiments, computer vision techniques and methods are utilized to identify a size, shape, symmetry, elevation, variability, color, and/or border of a lesion or mole. In some embodiments, computer vision techniques may identify diffusion of a lesion border from captured images of the lesion. Two or more images of lesions may be analyzed and compared using computer vision techniques to highlight moles or pigmentation which may be concerning due to characteristics which do not match other moles or pigmentation on an individual. In some embodiments, computer vision methods are utilized to compare images of a skin condition captured over a duration of time. Computer vision methods may be utilized to compare changes in, for example, size, shape, symmetry, elevation, variability, and/or color of a lesion or mole. Computer vision techniques may be able to produce more accurate analysis of changes in a lesion over time than achievable by a trained physician comparing images which may have been captured under different lighting.

In some embodiments, feature detection and extraction methods are utilized to identify a region of interest, such as a region of a skin condition or lesion. In some embodiment, feature detection and extraction methods comprise computing processing of images to analyze contrasts in pixel brightness to recognize features. Feature detection and extractions methods may include edge detection, corner detection, blob detection, ridge detection, and combinations thereof.

In some embodiments, an edge detection algorithm is utilized to identify an outline or border 425, 445 of a lesion or skin condition. In some embodiments, a nearest neighbor, thresholding, clustering, partial differential equation, and/or other digital image processing methods are utilized to identify an outline or border 425, 445 of a lesion or skin condition. In some embodiments, such diagnoses are performed by identifying and categorizing sections or pixels and then labeling or annotating each section/pixel as “normal skin” or “lesion skin.” Canny, Deriche, differential, Sobel, Prewitt, and Roberts cross edge detection techniques may be utilized to identify a region or border of a skin condition or lesion. In some embodiments, Gaussian or Laplacian techniques are utilized to smooth or improve the accuracy of the identified region or border of a skin condition or lesion. Edge detection may also be utilized to evaluate diffusion in the border of a lesion.

In some embodiments, segmentation of symmetry regions from normal skin, segmentation of asymmetry regions from normal skin, segmentation of pigment network regions from normal skin, segmentation of blotches from normal skin, segmentation of dots/globules from normal skin, and segmentation of dermatoscopic features such as: pigment network, amorphous structureless areas (blotches), leaf-like areas, blue ovoid masses, milia-like cysts, fissures and comedo-like openings, blood vessels, etc. are achievable using feature detection and extraction techniques.

Computer vision techniques, methods, and algorithms are implemented in a variety of ways to assess captured images of lesions or other skin conditions of interest. In some embodiments, the teledermatology platform, as shown in FIG. 15, includes a back-end system 1545, such as a cloud computing system or application, accessible via a network 1505. In further embodiments, the back-end system 1545 includes one or more computer vision modules 1546 each configured to apply one or more computer vision techniques, methods, and/or algorithms to captured images (e.g., photos or videos). Such computer vision module(s) 1546 may perform the feature identification, detection, extraction, and/or evaluation described herein (such as the edge detection described herein), the image comparison described herein, and/or the labeling or annotation described herein, etc. In a particular embodiment, one or more computer vision modules are implemented at an edge device, such as a patient device 1510 and/or a healthcare provider device 1520, such that computer vision techniques, methods, and algorithms can be performed in the local environment of the user.

In some embodiments, the image capture system or software includes a video conferencing solution. In some embodiments, video conferencing allows a telemedical operator, a physician, a licensed provider, or other healthcare professional to provide a live evaluation of one or more affected skin areas. In some embodiments, a video conference may include a 3-way conference to include a medical technician for documentation.

In some embodiments, the system provides remote control of a user device. A remote user may be able to initiate image capture using remote control of the user device, such that a successful image of a lesion or skin area of interest may be captured when the patient is unable to properly orient the camera of their device. For example, if a patient wishes to capture an image of a mole on their back, they may set their device on a stand pointed towards them and the remote user may initiate the capture of one or more images of the mole. Remote actuation of the device's camera may allow for a successful capture of images of the mole on the patient's back by a remote user, when the patient is unable to properly orient or view their device for image capturing.

In some embodiments, the image capturing system includes capture and evaluation features. In some embodiments, the image capturing system includes storage and data transmission of skin images. In some embodiments, the image capturing system includes mobile skin evaluation software. The skin evaluation software implemented may be MoleScope™ MoleMapper™, Miiskin™, or the like.

In some embodiments, the skin evaluation includes collection of patient information. In some embodiments, patient information is collected in a survey or questionnaire (provided, for example, by FIG. 15, 1518) completed by a patient, partner, caretaker, telemedical operator, a physician, licensed provider, or other healthcare professional. In some embodiments, the survey or questionnaire includes questions about a specific skin condition, lesion, or other health condition related to a skin condition to be assessed. The skin evaluation may further include evaluation of EMR, medical history, or the like. Additional information may come from spectroscopy, impedance measurements, protein and lipid analyses, and microbiome analyses.

A variety of skin conditions may be evaluated and tested using the telehealth solution described herein, including inflammatory skin conditions, skin cancer, skin conditions caused by pathogens (bacteria, viruses, fungi, protozoa, parasitic), immune mediated skin conditions, drug reactions, medical conditions where the skin is a surrogate target organ, wound healing/trauma (e.g., post-surgery), radiation damage, UV damage, and the like. In some embodiments, embodiments, the evaluation includes a skin cancer risk assessment (provided, for example, at a back-end system (see FIG. 15, 1545) at a skin condition evaluation module (see FIG. 15, 1547)).

In some embodiments, the skin evaluation includes a skin cancer evaluation as further described herein. In some embodiments, the skin care evaluation may be a melanoma evaluation as further described herein. In some embodiments, the skin evaluation is a skin cancer risk assessment.

In some embodiments the skin evaluation is a UV damage evaluation, as further described herein. In some embodiments, the skin evaluation is an inflammatory skin evaluation. In some embodiments, the skin evaluation is a psoriasis evaluation, as further described herein. In some embodiments, the skin evaluation is a skin health assessment. In some embodiments, the skin evaluation is a skin condition and/or age assessment. In some embodiments, the skin evaluation assesses microbiome flora on the skin.

In some embodiments, the skin evaluation provides an analysis or assessment of a skin condition. Following receipt of the analysis or assessment a patient may click on “order” or “need test” in system software application or module. In some embodiments, the user selecting an order command can trigger one or more captured images to be sent to a doctor for confirmation and/or approval, if needed.

### II. Ordering of a Skin Collection Kit

Provided herein are methods and systems for ordering of a skin sample or tissue sample collection kit. In some embodiments, the ordering is responsive to the evaluation of a skin condition. In some embodiments, provided herein, are systems and methods for distributing a skin or tissue collection kit. In some embodiments the sample collection kit is a non-invasive skin collection kit. In some embodiments, steps of ordering and approval of collection kits are carried out via modules (see e.g., FIG. 15, 1528) or features supported by software, such as a mobile or a web-based application.

In some embodiments, ordering of a skin sample or tissue sample collection kit is responsive to an approval or validation of an order for a collection kit based on the evaluation of a skin condition. In some embodiments, the approval or validation is conducted by a separate entity. In some embodiments, the approval or validation is conducted by a physician, licensed provider, or other healthcare professional.

In some embodiments, a method of ordering and/or approval of ordering of a collection kit includes steps of storing and forwarding images to a physician, licensed provider, or other healthcare professional. The method may be considered as store-and-forward (SAF) teledermatology.

In some embodiments, images and data collected from a skin evaluating are stored and forwarded to physician's device. In some embodiments, the physician's device is a mobile device allowing for reviewing and/or approval by the physician at any given location or time. In some embodiments, the information is provided to the physician's device as a banner message or graphical representation.

In some embodiments, an ordering module (see, e.g., FIG. 15, 1528) provides options for ordering a sample collection kit or follow-up procedure. Ordering options may include order tissue sampling kit, order consult, order follow up picture in 2-3 months. Ordering options may be provided for each image collected as part of a skin evaluation.

In some embodiments, orders are submitted by a patient, partner, or caretaker via a patient-facing software application or web-based application. In some embodiments, the orders are submitted through an ordering software feature or module of the application. In some embodiments, a user orders a follow-up procedure by selecting an option on a device interface. Options to order follow-up procedures or sampling kits may be available based specified workflow. In some embodiments, various ordering options are based on results of evaluation, insurance benefits, payer-based requirements, physician-recommendation, the like, or a combination thereof.

In some embodiments, a request to order requires physician and/or insurance approval. In some embodiments, a physician selects an option for approval on a physician-facing device interface through an ordering or approval software feature or module. In some embodiments, approval of one or more parties is required prior to distribution of a sample collection kit. In some embodiments, when evaluation data or a request to order a sample collection kit uploads, an email, banner notification, or other communication may be sent to a patient's physician and/or insurance representative. In some embodiments, images and information from the skin evaluation will be saved in their patient profile. The physician may select the communication and be linked to a module or software application. The module or software application may comprise a review portal. The physician may select the patient name and review the images. The physician may then use the provided information to determine if a skin or tissue sample needs to be collected. The provided information may include information collected during a skin evaluation, as disclosed above.

In some embodiments, a skin collection kit is ordered based from physician-facing software. In some embodiments, the ordering software feature or module allows a physician click on a link or otherwise interface with the software to order a follow-on procedure. In some embodiments, the follow-up procedure includes ordering of a skin or tissue sample collection kit. Insurance approval may be required from the app at the time of the request for test.

In some embodiments, follow-up procedures or sample collection kits are ordered automatically from patient-facing software based on the results of the evaluation step above. In some embodiments, the evaluation step includes image-analysis, patient information, medical history, insurance benefits, and/or the like. In some embodiments, the evaluation step includes automated evaluation of information by a programmed software feature or module.

In some embodiments, distribution of a sample collection kit is carried out by a professional courier service. In some embodiments, distribution of a sample collection kit includes automatic ordering, packaging, shipping, and or tracking. In some embodiments, a sample collection kit may be delivered by a drone. Drone delivery may limit contact and handling by persons to reduce contamination or health risks to a patient receiving the order.

In some embodiments, the sample collection kit is a non-invasive sample collection kit. Non-invasive tissue sample collection may utilize an adhesive patch, microneedles, skin brushes, scrapers, and the like.

In some embodiments, the teledermatological software application provides a guided tissue-sample collection feature. Clinician guided tissue-sample collection may be scheduled on demand or automatically to assist in proper sampling. In some embodiments, a clinician guided tissue-sample collection session is a video teleconference. In some embodiments, the video teleconference is carried out via the skin evaluation software or web-based application module.

In some embodiments, sample collection is performed by a certified technician. In such embodiments, the certified technician is scheduled to go to the home of a patient and collect the sample for the patient upon ordering of a skin or tissue sample. An on-site certification may enable sampling for elderly or those living alone when the lesion is not accessible by the patient.

### III. Testing and Reporting of Results

Provided herein are systems and methods for testing and reporting results of a skin tissue sample (see, e.g., FIG. 15, 1527). In some embodiments, the skin tissue sample is provided by a skin tissue sampling kit. In some embodiments, the sampling kit is a non-invasive skin tissue sampling. In some embodiments, sampling and tissue collection is done by a patient, partner or caretaker.

In some embodiments, results of a tissue sample test are sent to a physician-facing software/application. In some embodiments, results of a tissue sample test are sent directly to a patient-facing software/application. The results may be provided as a module or feature of a programmed software application or web-based application to provide and/or display the results of the testing. In some embodiments, results of a tissue sample test are sent to an electronic medical record (EMR). In some embodiments, results are transmitted directly from the testing lab to the patient, physician, and/or EMR.

In embodiments, where a results of a tissue sample test are sent to a physician-facing software/application, an alert may be sent to the physician when results are ready. The results may be provided as a module or feature of a programmed software application or web-based application to provide and/or display the results of the testing. The software or application may provide reminders to contact the patient regarding the results.

In some embodiments, results of a skin tissue sample analysis in combination with a skin evaluation, automatically triggering of follow-up actions. Follow-on actions may include scheduling an in-person appointment and/or biopsy. Automated actions may be triggered by specific sets of data gathered from the skin evaluation and sample testing. Automated actions may be carried out as a feature of a programmed software application or web-based application.

If it is determined that a skin sample collection error has occurred, another sampling kit may be sent out to the patient for re-sampling. Skin sample collection errors may include too little or no tissue sample received, contamination, or distribution errors. An additional sampling kit may be reordered by a lab, lab technician, physician, patient, caretaker, or other party involved in the collection or testing process.

In some embodiments, updates regarding the status of ordering, distributing, and/or testing of the tissue sample collection kit are provided. The updates may be provided via a software application or module. Communication or results and other information may be relayed by push notifications, emails, text messages, phone calls, or other communication means. In some embodiments, a physician-facing or patient-facing mobile device with a corresponding software application may display notifications or banner messaging to be reviewed anywhere and time. In some embodiments, updates may include a sample kits ordered, sample kits delivered, sample kits shipped, sample kits received by testing lap, testing complete, results reviewed, or other updates as to the status of the sample collection, distribution, and testing processes.

In some embodiments, algorithms, logic, and/or workflows may be used to produce a drug/procedure or additional test recommendations to the physician as part of the system and methods. Additional procedures may include options to order consult or order follow up picture in 2-3 months.

In some embodiments, result reports are generated and placed into a medical history file. In some embodiments, result reports are placed into the patient's EMR. In some embodiments, result reports may be sent to a medical billing agent. In some embodiments, the result reports are converted to an electronic file format such as a portable document format (PDF) to patients file and also sent to biller.

### IV. System to Connect Patients with Healthcare Specialists

In some embodiments, a system is provided to connect a patient with a healthcare professional for purposes of evaluating lesions, moles, or other skin anomalies. The healthcare professional may be a general physician, nurse practitioner, or dermatology specialist. The system may allow a patient or user to upload images and information corresponding to a skin area of concern to a network. The images may then be placed in a queue to be reviewed by a healthcare professional/provider who has registered with the network. In some embodiments, the healthcare professional is able to view the queue and select a submission for review. Based on the review, the healthcare provider can elevate the case and prescribe a PLA test to be sent to the patient.

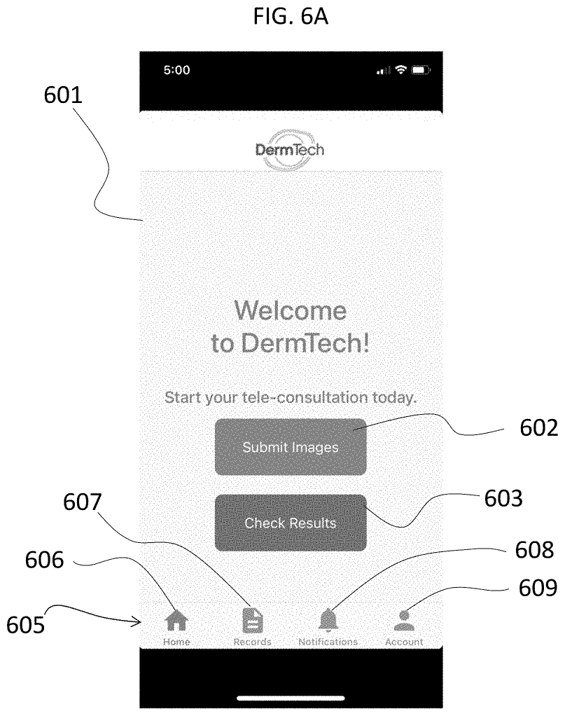

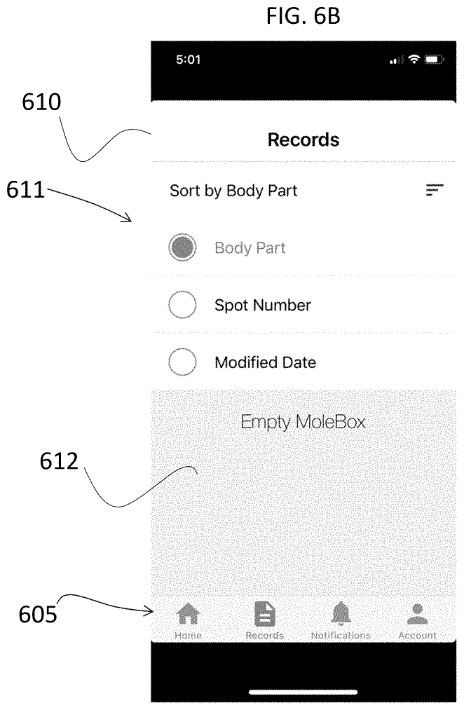

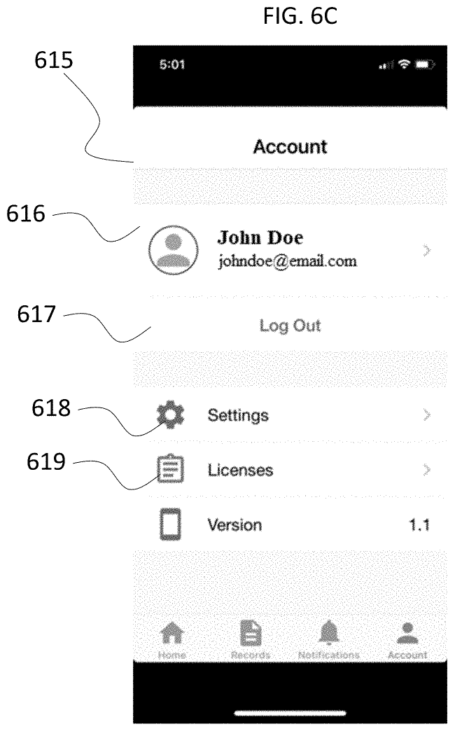

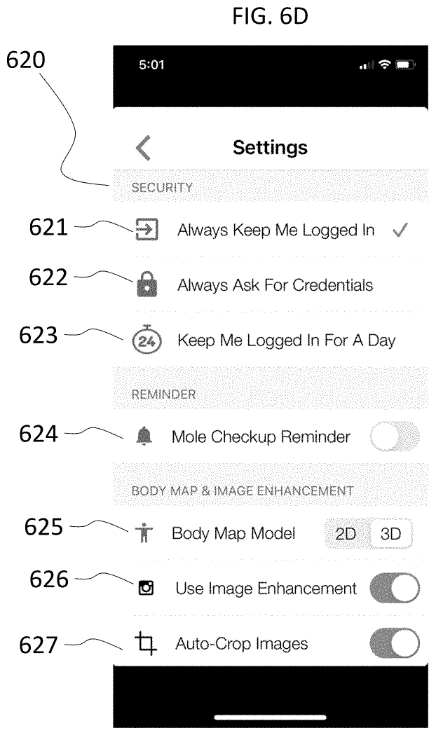

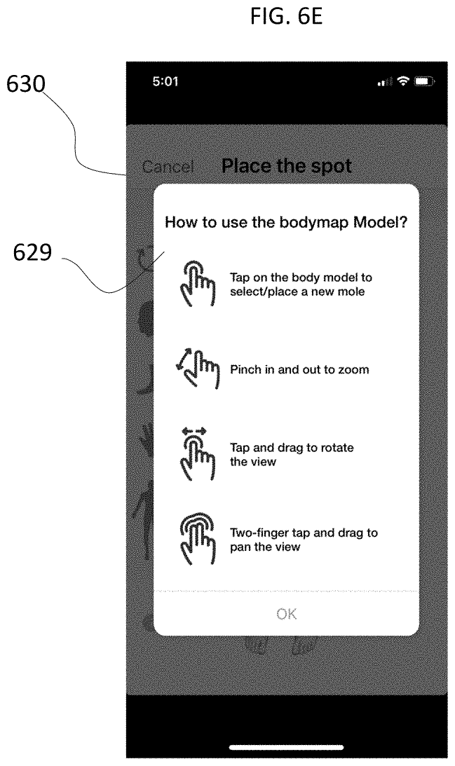

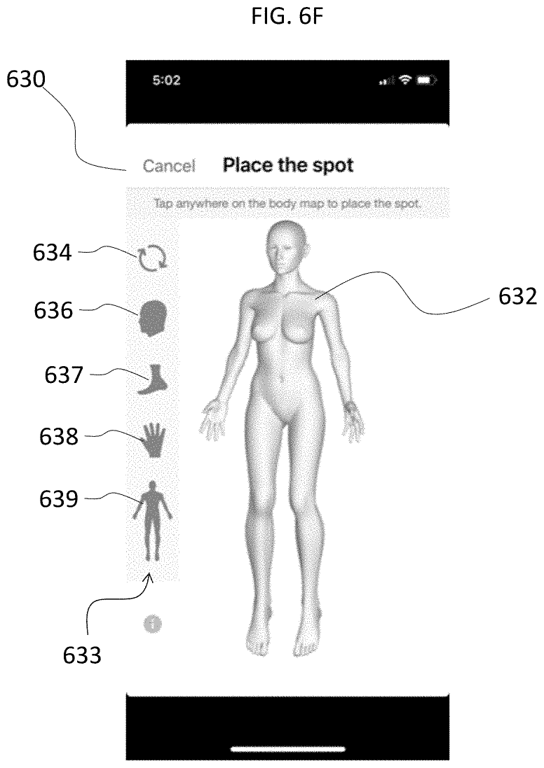

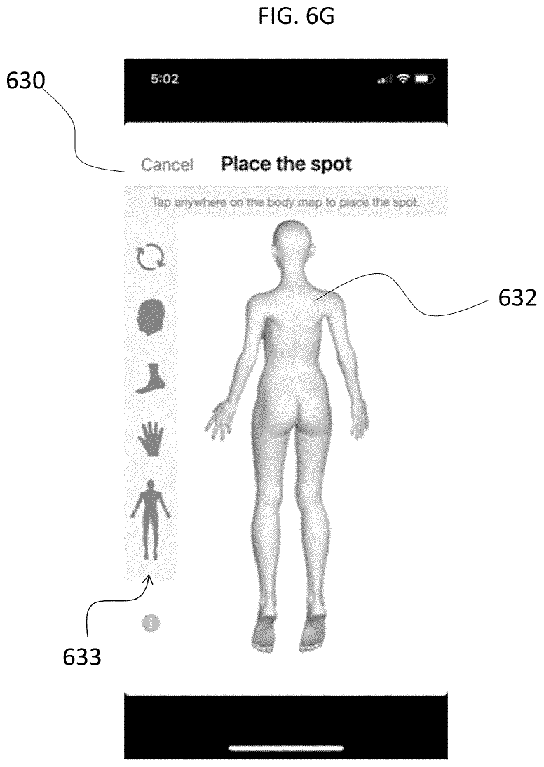

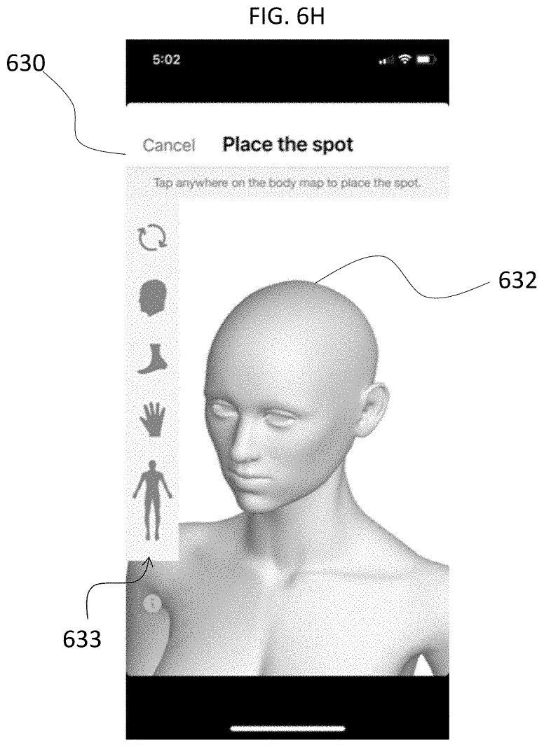

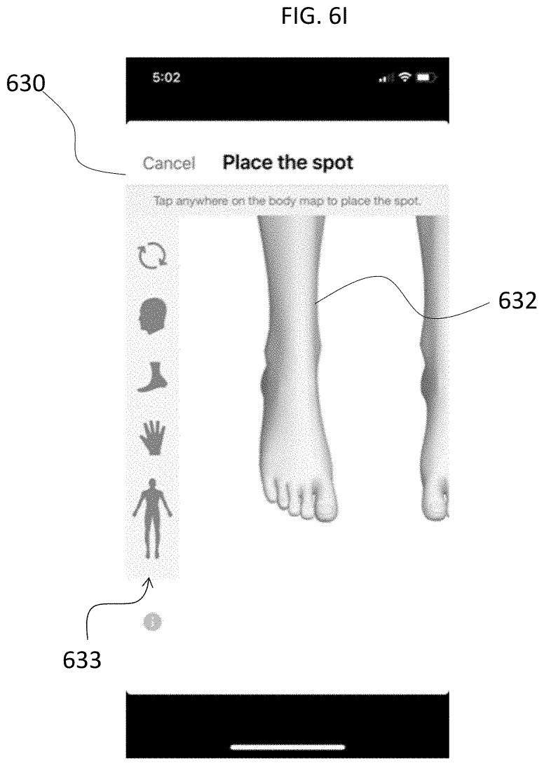

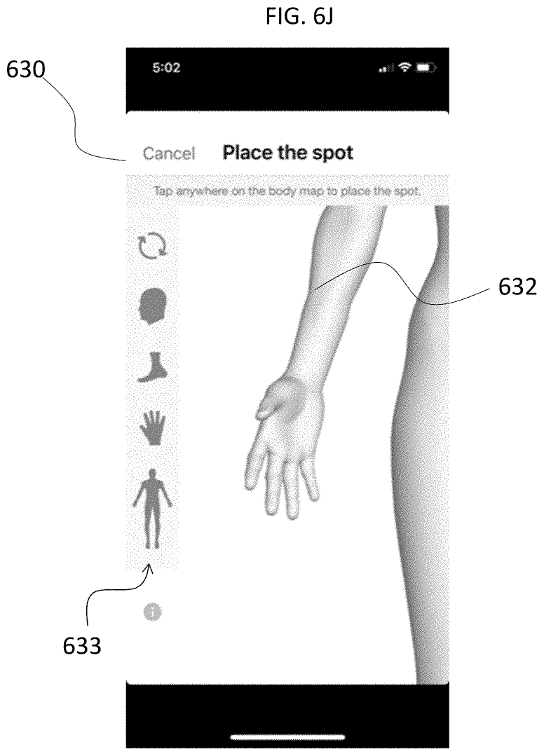

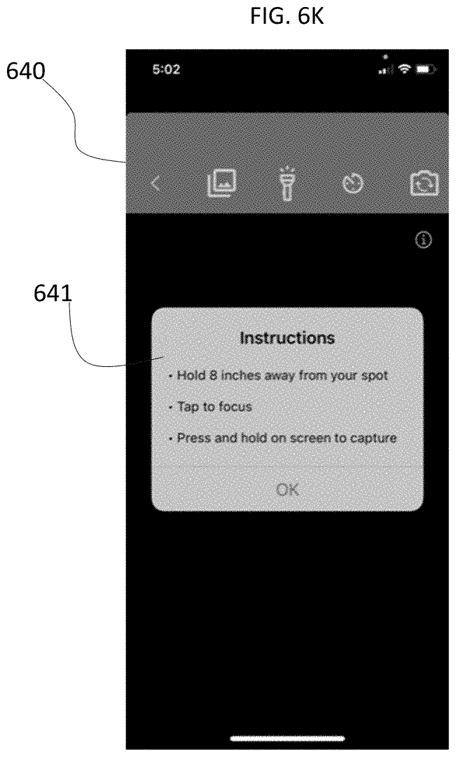

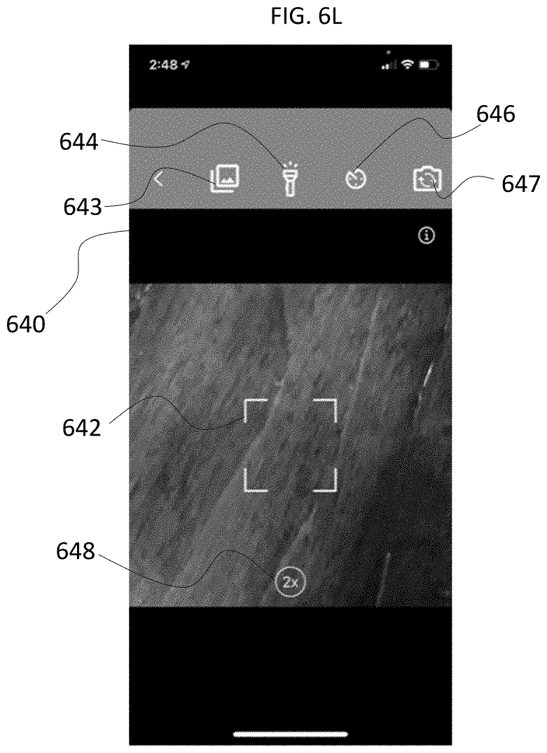

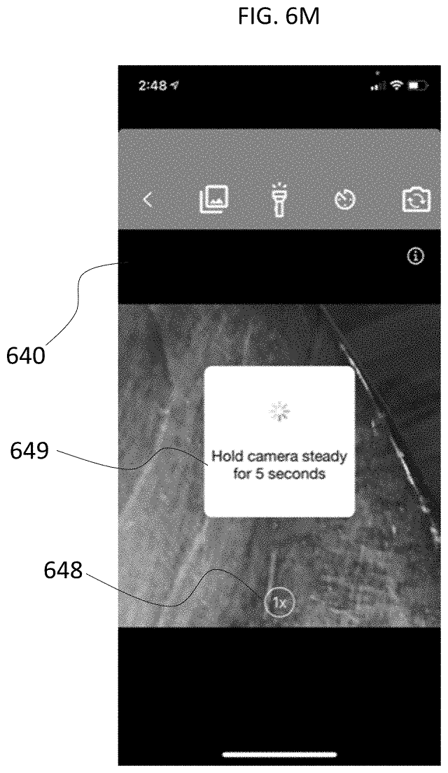

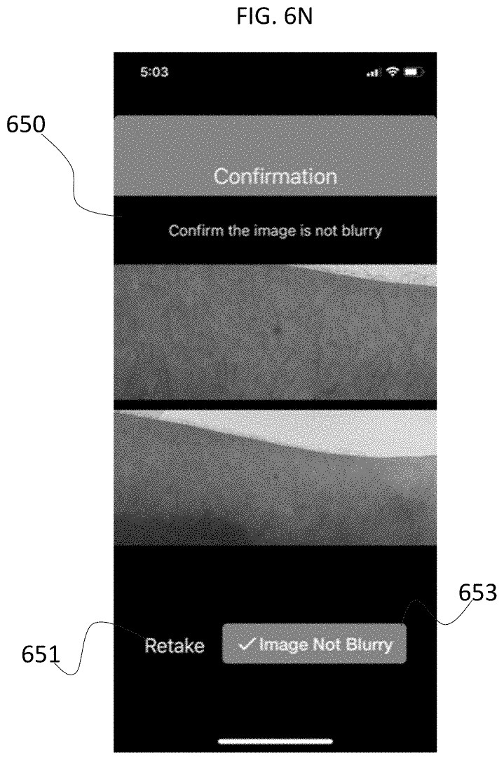

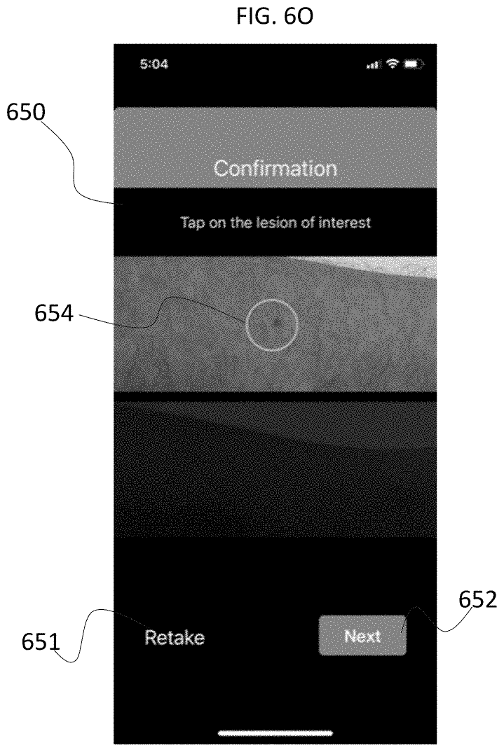

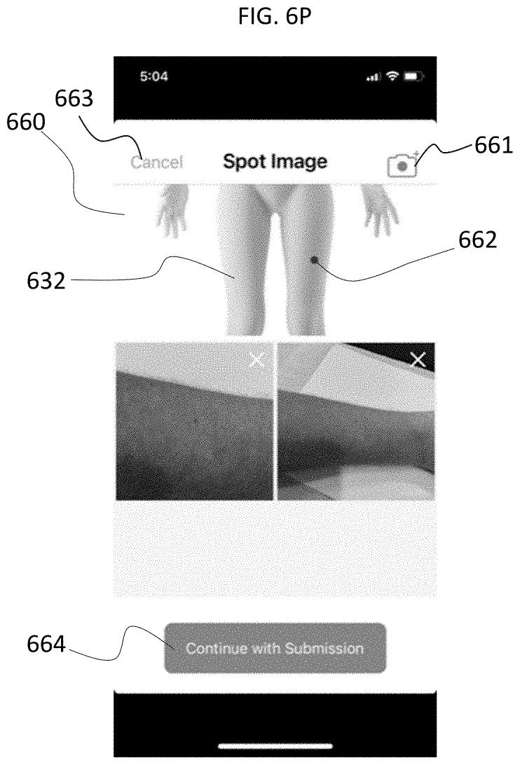

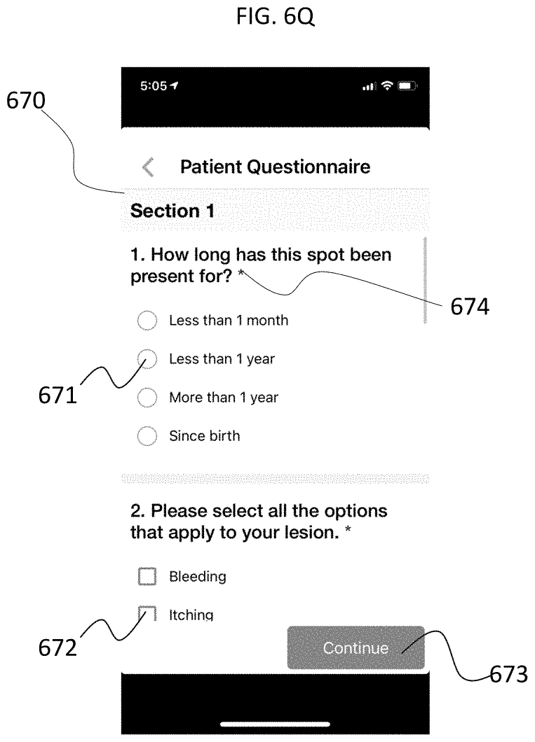

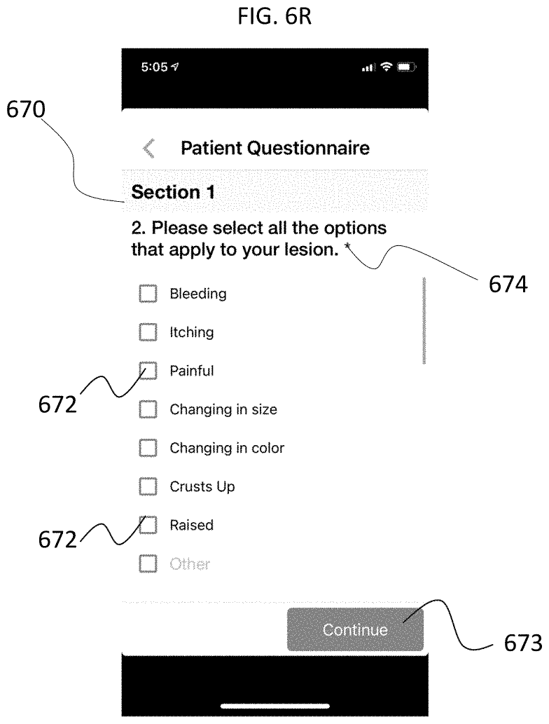

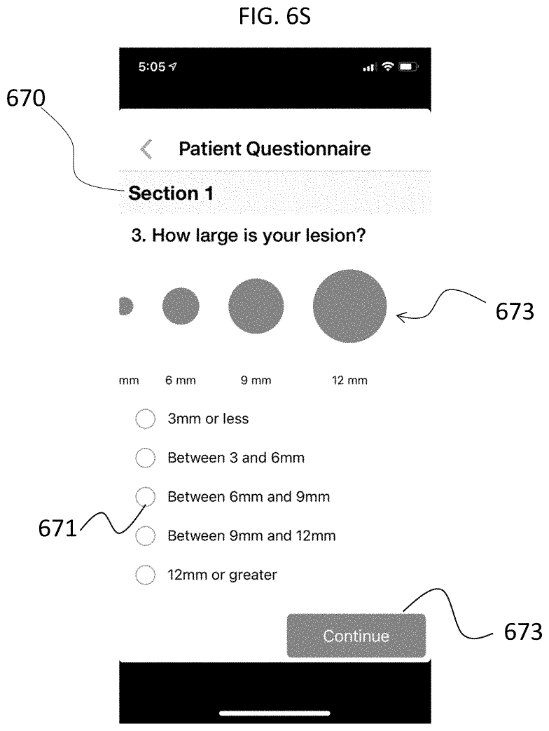

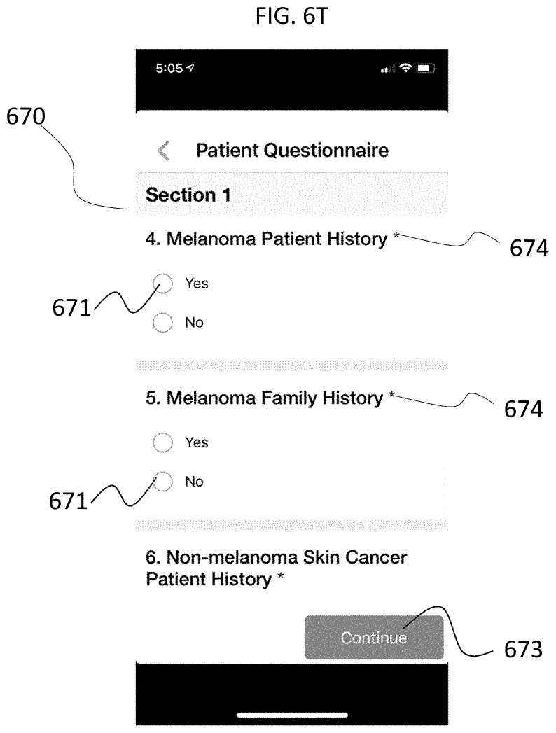

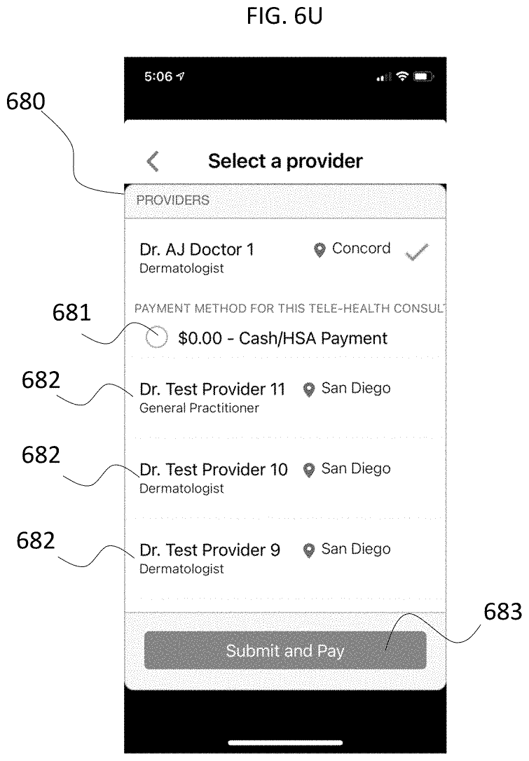

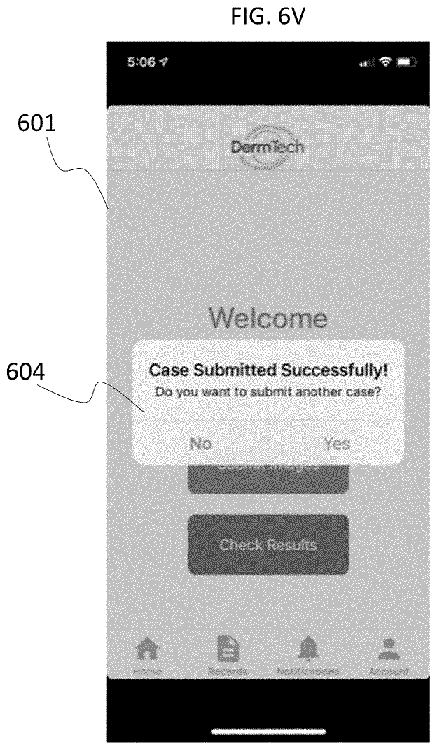

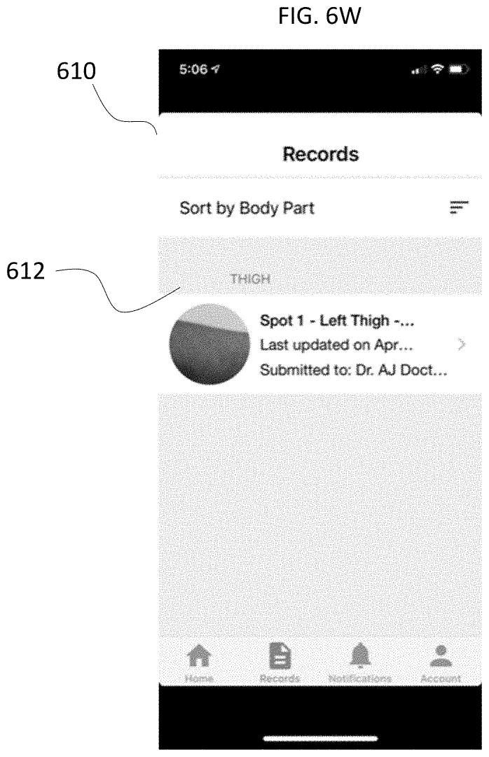

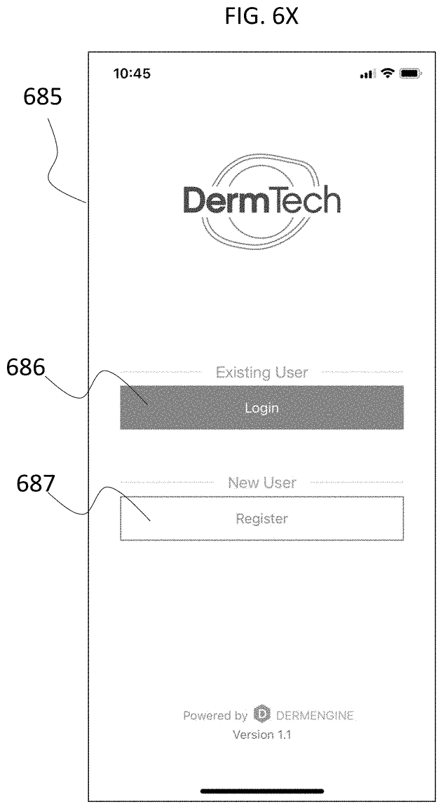

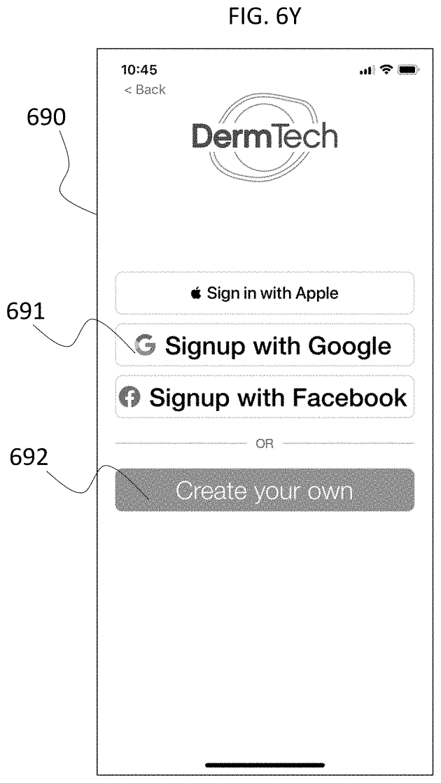

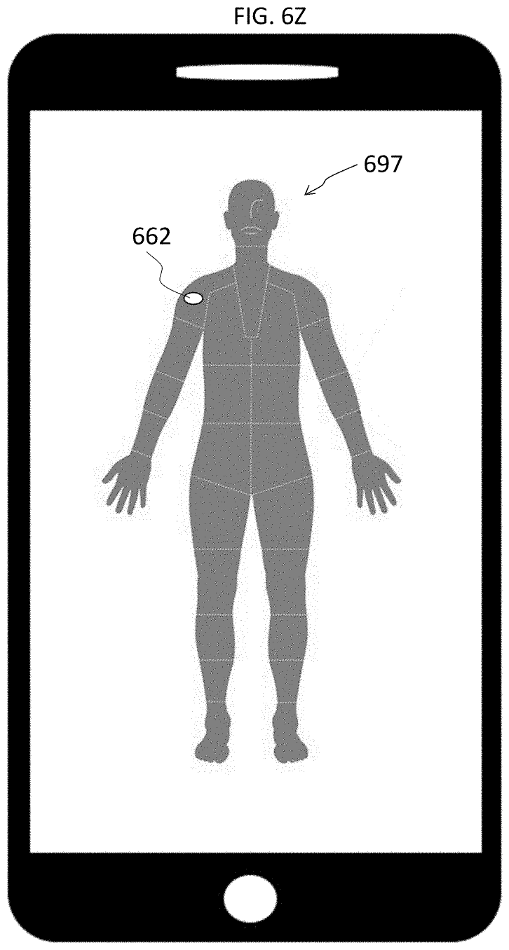

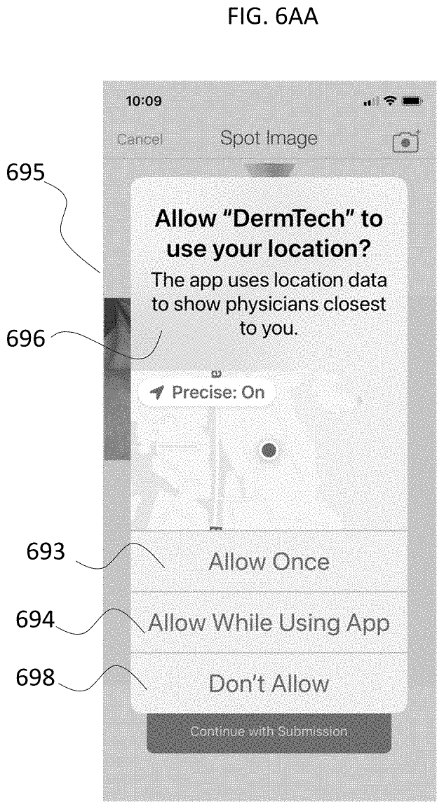

In some embodiments, the system allows for feedback from the user for reviewing the quality of health care they received. In some embodiments, cumulative feedback from users of the system is presented when the user goes to select a provider, e.g., at the provider selection user interface 680 (depicted in FIG. 6U). This implementation may further motivate the healthcare providers to quickly evaluate and provide feedback to the users and improve overall telehealth response times across the system.

A. Graphical User Interface/User Experience

With reference to FIGS. 6A-6V, exemplary graphical user interface/user experience (GUI/UX) designs are depicted. In some embodiments, the GUI/UX facilitates evaluation of a skin lesion, affected area, or region affected by a skin condition. In some embodiments, the GUI/UX facilitates evaluation of a skin lesion, affected area, or region affected by a skin condition via a software application. In some embodiments, the GUI/UX facilitates evaluation of a skin lesion, affected area, or region affected by a skin condition via a web-based application.

The following will be given with reference to a device that detects inputs on a touch-sensitive surface display. In some embodiments, the device detects contact with the touch-sensitive display at locations that correspond to respective locations on the display. In this way, user inputs detected by the device on the touch-sensitive display are used by the device to manipulate the user interface on the display. In some embodiments, the device includes one or more contact intensity sensors for detecting intensity of contacts on a touch-sensitive display. In some embodiments, the device comprises one or more tactile output generators generating tactile outputs for a user of device. It should be understood that similar methods are, optionally, used for other user interfaces described herein.

Additionally, while the following examples are given primarily with reference to finger inputs (e.g., finger contacts, finger tap gestures, finger swipe gestures, etc.), it should be understood that, in some embodiments, one or more of the finger inputs are replaced with input from another input device (e.g., a mouse based input or a stylus input), or input of another type, on the same device (e.g., a button press). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

In some embodiments, a predefined set of functions that are performed through a touchscreen and/or a touchpad. In some embodiments, functions are performed using one or more input devices, such as a mouse and keyboard. In some embodiments, functions include navigation between user interfaces. In some embodiments, the touchpad, when touched by the user, navigates device to a main, home, or root menu from any user interface that is displayed on the device. In some embodiments, other embodiments, the menu button is a physical push button or other physical input control device instead of a touchpad or touchscreen input. In some embodiments, the electronic device is in communication with a separate remote control through which it receives user inputs (e.g., the remote control includes a touch-sensitive surface or a touchscreen through which a user interacts with the electronic device).

FIG. 6A, illustrates a home screen user interface 601 on a device with a touch-sensitive display, according to some. In some embodiments, one or more navigational objects 605 are provided for navigation between user interfaces. In some embodiments, the one or more navigation objects comprise one or more virtual buttons which are selectable by a user. In some embodiments, one or more navigational objects 605 are provided on multiple user interfaces of the software application to facilitate navigation.

In some embodiments, the navigation objects 605 include a “home” button 606. The “home” button may be selected by a user to return to the home screen user interface 601. In some embodiments, the navigation objects 605 include a “records” button 607. The “records” button 607 may be selected by a user to navigate to the records user interface 610. In some embodiments, the navigation object 605 include a “notifications” button 608. The “notifications” button 608 may be selected by a user to navigate to a notifications user interface. In some embodiments, the notifications user interface displays alerts, notifications, and/or messages regarding status of an evaluation or lab results. In some embodiments, the navigation objects 605 include an “account” button 609. The “account” button 609 may be selected by a user to navigate to the account user interface 615.

In some embodiments, the home screen user interface 601 comprises one or more user input objects. In some embodiments, the user input object comprises one or more virtual buttons which are selectable by a user. In some embodiments, the user input objects include a “submit images” button 602. In some embodiments, the submit images button 602 is selectable by a user to begin an image capture (utilizing, for example, FIG. 15, 1516), mapping (utilizing, for example, FIG. 15, 1517), and transmission procedure, as described herein. The image capture, mapping, and transmission procedure may be designed to capture images and information corresponding to a lesion or region of interest. Through a series of graphic user interfaces to present a user experience, data corresponding to a lesion or region of interest may be assembled into a submission package or case for submission to a physician for examination (at, for example, FIG. 15, 1526). In some embodiments, the user input objects include a “check results” button 603. In some embodiments, the “check results” button is selectable to display results, including lab results or physician evaluations, from previously submitted evaluations.

FIG. 6B illustrates a records user interface 610 on a device with a touch-sensitive display. In some embodiments, one or more navigational objects 605 are provided on the records interface 610 for navigation between user interfaces.

In some embodiments, the records user interface 610 comprises one or more selectable radio buttons 611. In some embodiments, the radio buttons are selectable to sort records of previously submitted evaluation packages in the records display window 612. FIG. 6B illustrates a records display window 612 prior to submission of any evaluation packages. FIG. 6X depicts a records display window 612 after submission of an evaluation packages. In some embodiments, an evaluation package refers to the captured images of a region of interest affected by a skin condition, or a lesion, combined with submitted information used to describe the region of interest affected by a skin condition, or the lesion.

In some embodiments, the radio buttons 611 allow a user to sort the evaluation package records by body part, by spot number, or by modified date. In some embodiments, only one radio button is selectable at a time. In some embodiments, the spot number refers to the order in which the evaluation package was submitted, i.e., a first evaluation package corresponding to images of a first lesion or skin condition and the corresponding data would be assigned spot number one, and a second evaluation package corresponding to images of a second lesion or skin condition and the corresponding data would be assigned spot number two. In some embodiments, wherein images and/or evaluation packages of the same lesion, affected area, or spot are captured at two different times, evaluation package submitted at the later date will be assigned the same spot number.

FIG. 6C, illustrates an account user interface 615 on a device with a touch-sensitive display. In some embodiments, one or more navigational objects 605 are provided on the account interface 610 for navigation between user interfaces.

In some embodiments, the account interface comprises a user information window 616. In some embodiments, the user information window displays the name of a user logged into the application. In some embodiments, the user information window displays a recorded email address of the user logged into the application. In some embodiments, the user information window is selectable. In some embodiments, selection of the user information window navigates the user to a page to edit user information.

In some embodiments, the account user interface comprises a selectable “log out” button 617. In some embodiments, selection of the “log out” button 617 exits the application, such that input of user information and/or a password is required to log into the application and access data associated with a user. In some embodiments, the log out button 617 returns the user to a login user interface (690 as depicted in FIG. 6Y).

In some embodiments, the account user interface 615 further comprises a selectable “settings” button 618 for navigation to a settings user interface 620. In some embodiments, the account user interface 615 further comprises a selectable “licenses” button 619 for displaying information of the licenses associated with the application. In some embodiments, the account user interface 615 displays the installed version of the application. In some embodiments, the account user interface 615 provides a selectable “version” button to navigate to a new interface for updated the version of the application. In some embodiments, a selectable “version” button opens a new software application loaded onto the device for updated the application. The software application for updating the skin evaluation software application may be an app store, e.g., Google Play.

FIG. 6D, illustrates a settings user interface 620 on a device with a touch-sensitive display, according to some embodiments. In some embodiments, the setting user interface comprises one or more user input objects associated with security settings. In some embodiments, one or more selectable security setting buttons allow for toggling of security preferences. In some embodiments the one or more selectable security buttons comprise an “always keep me logged in” button 621, a “always ask for credentials” button 622, and/or a “keep me logged in for a day” button 623. In some embodiments, an “always keep me logged in” button 621 is selectable such that a user does not need to enter credentials upon opening the software application. In some embodiments, an “always ask for credentials” button 622 is selectable such that a user will need to enter credentials upon every instance of opening the software application and/or upon every instance of locking the device which the software application is loaded on. In some embodiments, if the always ask for credentials button 622 is selected the user will return to a login user interface (685 as depicted in FIG. 6X) each time the application is closed, the device is locked, and/or the device is powered off. In some embodiments, a “keep me logged in for a day” button 623 is selectable such that a user does not need to enter credentials for a duration of 24 hours after an instance of entering credentials to log into the application.

In some embodiments, the settings user interface comprises one or more user input objects associated with reminder settings. In some embodiments, a reminder toggle switch 624 is provided to turn on or turn off notifications associated with reminders to submit evaluation packages for lesions or affected skin areas which should be monitored. In some embodiments, the notifications are device notifications (e.g., bubble or pop-up notifications), email notifications, phone notifications, and/or text notifications (e.g., SMS or MMS messages).

In some embodiments, the setting user interface comprises one or more user input objects associated with body map and image enhancement settings. In some embodiments, a body map model toggle switch 625 is provided to toggle between an avatar having a two-dimension representation (697 as depicted in FIG. 6Z) or a three-dimensional representation. In some embodiments, an image enhancement toggle switch 626 is provided to turn on or off image enhancement features, such as automated image brightening. In some embodiments, an auto-crop images toggle switch 627 is provided to turn on or off an auto-crop feature configured to automatically crop images one or more images of a lesion or affected region of skin after the images are captured.

In some embodiments, after selection of the “submit images” button on the home user interface 601, location marker interface 630 is displayed. In some embodiments, a gesture information user interface 629 is presented as a user lands on the location marker interface 629. In some embodiments, the gesture information user interface 629 displays a window comprising the acceptable inputs for controlling a virtual avatar (632 as depicted in FIGS. 6F-6J). In some embodiments, touchscreen gestures to manipulate a virtual avatar include tapping on the avatar to select a location of a lesion or affected skin region, pinching to zoom in or out of the avatar, taping and dragging to rotate the view of the avatar, and two-finger dragging to pan the view of the avatar. In some embodiments, tapping and dragging to rotate the view of the avatar is only available if the 3D model of the avatar is being utilized.

FIGS. 6F-6J depict an avatar user interface 630 (provided, for example, by FIG. 15, 1517), according to some embodiments. In some embodiments, the avatar user interface 630 displays a virtual avatar 632 to provide a representation of a human body. As discussed herein, the avatar 632 may be manipulated and regions of the avatar may be selected to place a location where an affected skin area is being captured. In some embodiments, the avatar user interface 630 includes one or more quick manipulation buttons 633 for quickly manipulating the avatar and facilitate placement of a lesion location. In some embodiments, the manipulation buttons comprise a flip button 634 to quickly flip the avatar 180 degrees about a front plane. In some embodiments, the manipulation buttons comprise a head zoom button 636 to quickly zoom into the head region of the avatar. In some embodiments, the manipulation buttons comprise a feet zoom button 637 to quickly zoom into region near the feet of the avatar. In some embodiments, the manipulation buttons comprise a hands zoom button 638 to quickly zoom into region near the hands of the avatar. In some embodiments, the manipulation buttons comprise a full profile button 639 to quickly zoom out to display the full body of the avatar.

FIG. 6F depicts a front-facing view of the avatar 632, according to some embodiments. In some embodiments, this view is the default view of the location marker interface 630. In some embodiments, a user can quickly switch to this view by selecting the full profile button 639.

FIG. 6G depicts a rear-facing view of the avatar 632, according to some embodiments. In some embodiments, a user can quickly switch to this view by selecting the full profile button 639, then the flip button 634. In some embodiment, a user can quickly switch to this view by selecting the full profile button 639 twice.

FIG. 6H depicts a zoomed in view of the head of the avatar 632, according to some embodiments. In some embodiments, a user can quickly switch to this view by selecting the head zoom button 637.

FIG. 6I depicts a zoomed in view of a foot of the avatar 632, according to some embodiments. In some embodiments, a user can quickly switch to this view by selecting the feet zoom button 638.

FIG. 6J depicts a zoomed in view of a hand of the avatar 632, according to some embodiments. In some embodiments, a user can quickly switch to this view by selecting the hand zoom button 639.

After a location of an affected skin area is selected, an image capturing user interface 640 is provided (provided, for example, by FIG. 15, 1516), according to some embodiments. FIG. 6K depicts an image capturing user interface 640 and instruction window 641, which provides instructions for the successful capture of image of the affected skin area, according to some embodiments. In some embodiments, the instructions direct a user to hold the device/camera about 8 inches away from the affected area, spot, or lesion; tap the touchscreen of the device to focus the image; and press and hold the touch screen to capture the image or images of the affected area. In some embodiments, the instruction window 641 is displayed for a predetermined amount of time. In some embodiments, the instruction window 641 is displayed until the user taps the screen.

FIG. 6L depicts an image capturing user interface 640, according to some embodiments. In some embodiments, the image capturing user interface 640 comprises a reticle 642 to facilitate placement of the target spot within the frame of the image to be captured. In some embodiments, the image capturing user interface 640 comprises an upload button 643 to select an image which is stored on the device's memory for uploading to the software application. In some embodiments, the image capturing user interface 640 comprises a light button 644 to turn on the device's flashlight. In some embodiments, the image capturing user interface 640 comprises a timer button 646 for setting a timer to capture an image using the device. In some embodiments, the image capturing user interface 640 comprises a camera flip button 647 to flip between the rear- and front-facing cameras of the device. In some embodiments, the image capturing user interface 640 comprises a zoom indicator 648 for displaying the current zoom level of the camera. The zoom indicator 648 may be selectable to adjust the zoom level of the camera.

FIG. 6M depicts a capturing user interface 640 after picture capture has been initiated, according to some embodiments. In some embodiments, after a user presses and holds the touchscreen of the device to initiate an image capture sequence, a prompt 649 is displayed instructing the user to hold the camera steady. In some embodiments, the software application utilizes the devices camera to capture two images of the region of interest. In some embodiments, the images are captured sequentially. In some embodiments, the images are captured simultaneously.

FIG. 6N depicts a confirmation user interface 650, according to some embodiments. In some embodiments, the confirmation user interface 650 displays one or more images showing the affected skin area as captured. In some embodiments, the confirmation user interface 650 displays an image showing the affected skin area which is zoomed in and cropped. In some embodiments, the confirmation user interface 650 displays the zoomed in image next to the image captured without zoom or magnification. In some embodiments, the confirmation user interface 650 comprises one or more selectable buttons. In some embodiments, the selectable buttons comprise a “retake” button 651 provided to navigate back to the image capturing user interface 640 if the user confirms that the captured images are blurry. In some embodiments, the selectable buttons comprise a “image not blurry” confirmation button 653 provided to continue to the lesion highlighting procedure if the user confirms the captured images are not blurry. Automated or cloud-based verification of quality image captures may be used to verify that captured images are not blurry.

FIG. 6O depicts the confirmation user interface 650 during a lesion highlighting procedure, according to some embodiments. In some embodiments, a user taps on the captured image to highlight the lesion of interest with an outline 654. In some embodiments, outline 654 is displayed a circle. In some embodiments, outline 654 is displayed an oval, square, triangle, or other suitable geometric shape. If the user is satisfied with the quality of the image, they may select the “next” button to continue to an image review user interface 655. In some embodiments, outline 654 is automatically applied to automatically applied. In some embodiment, computer vision techniques are utilized to identify the lesion of interest and apply the outline 654. In some embodiments, a user verifies the correct placement of the outline 654.

FIG. 6P depicts an image review user interface 660, according to some embodiments. In some embodiments, the image review user interface 660 displays the avatar 632 with the lesion location 662. In some embodiments, the image review user interface 660 displays the images captured by the software application. In some embodiments, the image review user interface 660 displays both the regular and zoomed in images captured by the software application. In some embodiments, if a user is not satisfied with the captured images a camera button 661 is provided to reload the image capturing user interface 640. In some embodiments, if a user is not satisfied with the marked lesion location 662 or wants to quite the submission process, a “cancel” button 663 is provided as part of the image review user interface 660. If the user is satisfied with the captured image and the marked location 662, the “continue with submission” button 664 may be selected to provide a questionnaire user interface 670.

FIGS. 6Q-6T depict a questionnaire user interface 670, according to some embodiments. In some embodiments, the questionnaire user interface presents a series of questions to obtain information from a user corresponding to the lesion or affected area, of which the images were captured using the camera of the device. In some embodiments, the questionnaire user interface 670 comprises one or more radial buttons 671 for selecting a single answer to a presented question. In some embodiments, the questionnaire user interface 670 comprises one or more checkboxes 671 for possible selection of multiple answers to a presented question.

In some embodiments, the questionnaire user interface 670 comprises a “continue” button 673 for continuing on to the provider selection user interface 680. In some embodiments, a designation 674 is used to mark questions which are required before proceeding to the provider selection user interface 680. In some embodiments, the designation comprises a red asterisk.

As depicted in FIG. 6S, one question presented by the questionnaire user interface 670 may require a user to input the size of the lesion of interest. In some embodiments, the selectable sizes comprise 3 millimeters (mm) or less, 6 mm, 9 mm, and 12 mm or greater. In some embodiments, a size key 675 is provided to facilitate estimation of the size of the lesion of interest. In some embodiments, the size key 675 displays reference images which are to scale or a 1:1 of the size being identified.

FIG. 6U depicts a provider selection user interface 680, according to some embodiments. In some embodiments, the provider selection user interface 680 provides one or more selectable provider buttons 682 for choosing eligible providers. In some embodiments, eligible providers are listed and/or sorted by distance to a user's location or selected location. In some embodiments, the software applications interfaces with an insurance provider network, as described herein, such that only providers compatible with the user's insurance are displayed. In some embodiments, a selected provider is displayed at the top of the selection user interface 680. In some embodiments, the selection user interface 680 comprises one or more radial buttons 681 to allow a user to select a payment method. In some embodiments, the software applications interfaces with an insurance provider network, as described herein, and the cost of the selectable payment methods is displayed. In some embodiments, the selection user interface 680 provides a “submit and pay” button 683 for submission of the payment information, images, and data collected by the software application. Upon submission the home screen user interface 601 is reloaded.

In some embodiments, with reference to FIG. 6V, the home screen user interface 601 may provide a pop-up window 604 confirming the submission of the images and information. In some embodiments, the pop-up window 604 provides a question to inquire if the user wishes to submit another case.

FIG. 6X depicts a login user interface 685, according to some embodiments. In some embodiments, the login user interface comprises a selectable “login” button 686 and a selectable “register” button. In some embodiments, selection of the “login” button 687 loads a user credential interface for a user to enter their credentials to log into the software application. In some embodiments, selection of the “register” button 687 loads a user registration interface 690.

FIG. 6Y depicts a registration user interface 690, according to some embodiments. In some embodiments, the registration user interface 690 comprises one or more selectable linked registration buttons 691 for linking an existing user account with the software application. An existing user account may be a social networking account, an email account, or other database account. In some embodiments, the registration user interface 690 comprises a selectable custom registration button 692. In some embodiments, upon selection of the custom registration button 692, the registration user interface 690 provides fields for a user to create credentials for the software application.

As depicted in FIG. 6AA, a user interface 695 of the application may display a pop-up window 696 requesting that the software application access the devices location hardware to identify a current location of the patient. The user may select the “allow once” button 693 to allow the application to access the user location for just the current instance. The user may select the “allow while using app” button 694 to allow the application to access the user location whenever the software application is in use. The user may select the “don't allow” button 698 to deny the application to access the user location.

B. Teledermatology Platform

In some embodiments, as depicted by FIG. 15, a user connects to a network 1505 using with a patient-facing device 1510. The network may comprise one or more websites to load a patient user interface (UI) 1515 to the patient-facing device 1510. In some embodiments, the patient UI 1515 comprises, by way of examples, imaging features (provided, for example, by an image module 1516), mapping features (provided, for example, by a mapping module 1517), questionnaire/survey features (provided, for example, by a question module 1518), and the like. In some embodiments, the patient UI 1515 comprises a selectable button or link which allows the user to request a skin evaluation. An initial skin evaluation may comprise an image review of a skin lesion, mole, or other skin anomaly by a healthcare professional.

If the patient has not used the system before, she/he may be prompted to register by inputting their information. In some embodiments, registration information may comprise the name, address, email, and phone number of the patient. In some embodiments, registration comprises consenting to one or more user agreements, which may include information data sharing agreements, privacy agreements, and the like. In some embodiments, the user consents to allowing the system to access one or more hardware devices of the patient-facing device 1515.

In some embodiments, upon registering and requesting a skin evaluation the system provides a cost estimate for the image review. In some embodiments, the user is provided with a list of selectable insurance providers, and the system requests that the user selects the patient's insurance provider. In some embodiments, the user inputs the patient's insurance information and the system connect to a database 1530 of the selected insurance provider to retrieve the insurance information for the patient. In some embodiments, the user captures one or more images of the insurance information or card and uploads the images to the network 1505 using the patient-facing device 1510. In some embodiments, based on the information retrieved from the insurance database 1530, the network 1505 provides a cost of the image review to the user.

In some embodiments, network 1505 requests that a patient or user provides payment information. The user may input payment information into the patient user interface 1515 to be received by the network. In some embodiments, the payment information is stored in the network for future use. Payment information may comprise credit card information, debit card information, bank information, or the like.

In some embodiments, after the payment information is received, the user is asked if they are ready to capture images of the lesion, mole, or other skin anomaly. In some embodiments, the user inputs a mobile phone number, and the network sends a link to the mobile phone associated with the number. The link may be selected by the patient to begin the image collecting process.

In some embodiments, the selected link loads a cloud based software application onto the patient user interface 1515. In some embodiments, the cloud based software application mirrors a software application which is loaded onto a patient-facing device, as disclosed herein. In some embodiments, the software application guides the user in capturing one or more images of the affected skin area or skin anomaly. Steps for capturing one or more images of the affected skin area or skin anomaly may be the same or similar to the steps utilized by the software application disclosed herein. In some embodiments, the user is prompted to select the area of the body on which the skin anomaly is located on an avatar (e.g., avatar 632 depicted in FIGS. 6F to 6J). In some embodiments, the user is prompted to upload one or more images of the skin anomaly using the patient-facing device 1515. In some embodiments, the user is prompted to capture one or more images of the skin anomaly using a camera of the patient-facing device 1515. In some embodiments, the user is prompted hold the camera away from lesion and press and hold anywhere on screen. In some embodiments, the user is prompted to hold the camera approximately 8 inches away from the skin anomaly. In some embodiments, the system captures two images simultaneously (normal and close up views).

In some embodiments, the user is asked if the captured images appear blurry. If the user selects that the images appear blurry, the user may be prompted to retake the images. In some embodiments, the user is asked to highlight the area of concern for the captured images. In some embodiments, the user creates circle around the area of concern.

After the images are collected and the area of concern is highlighted, the user may be asked if the images are ready to upload. If yes, patient may upload the images onto the network. In some embodiments, one or more fields are provided for the user to input information corresponding to each uploaded image of an area concern. In some embodiments, the user is asked how long the lesion has been there. In some embodiments, the user is asked to input the size of the lesion. In some embodiments, the user is prompted to provide a list of concerns about the area of concern. In some embodiments, an open field is provided for the user to input any additional comments. In some embodiments, the user is asked if the images are ready for submission. In some embodiments, the user consents being billed using the submitted payment information.

In some embodiments, if a user begins a submission for an image review process, but does not complete and submit the images, then a reminder email is automatically sent to the user. In some embodiments, the reminder email comprises a link to direct the user the incomplete submission.

In some embodiments, the images are sent to an image review queue, and the user receives confirmation that the images have been successfully submitted. In some embodiments, the confirmation is an email confirmation. In some embodiments, the confirmation in includes an approximation of when a response should be received.

In some embodiments, the system comprises a plurality of health care providers which have opted in or registered to review images uploaded to the network by users of the system. In some embodiments, a health care provider enters their information as part of the opt-in process. In some embodiments, a healthcare provider registers a financial account for receiving payment. In some embodiments, payment is sent to the healthcare provider based on the number of images reviewed. Payments may be made and deposited into the financial accounts of the registered healthcare providers on a regular basis, such as a weekly, bi-weekly, or monthly basis. In some embodiments, the healthcare provider selects how often they wish to receive a payment.

In some embodiments, the healthcare provider registration includes registering into a network referral system. In some embodiments, the network referral system connects patients with local healthcare providers. In some embodiments, the network only allows healthcare providers within proximity of the user location to review the images uploaded by the user. In some embodiments, a user or patient may user the network referral system to find a healthcare provider close to their location. In some embodiments, a user sets a range which they are willing to travel for an appointment with a healthcare provider. In some embodiments, the user location is an address provided by the user. In some embodiments, the user location is provided by a global positioning system of a user or patient-facing device.

In some embodiments, a healthcare provider is notified when an image evaluation has been requested by a patient within proximity to the location of the healthcare provider. In some embodiments, requests from patients within proximity to the location of the healthcare provider are provided in a queue. In some embodiments, a healthcare provider is able to select a user uploaded submission, including images of the area of concern. In some embodiments, the uploaded images and user submitted fields are displayed on a healthcare provider UI 1525 loaded onto a healthcare provider-facing device 1520 provided by the network 1505. In some embodiments, the healthcare provider UI 1525 comprises, by way of examples, virtual visit review features (provided, for example, by a visit review module 1526), skin sample kit results review features (provided, for example, by a results module 1527), skin sample kit ordering features (provided, for example, by an ordering module 1528), and the like.

In some embodiments, if the healthcare provider deems the images are too blurry the healthcare provider submits a request for the user to retake images of the area of concern. The request to retake the images may be sent to the patient or user in an email. The email may contain a link to load a patient UI and retake images of the area of concern using the foregoing process.

In some embodiments, a healthcare provider is able to submit that the area of concern is clearly benign, and no follow-up is required. The system may generate an email to the patient to confirm that the area of concern is benign.

If the healthcare provider determines that the area of concern is questionable, the healthcare provider may prescribe a pigmented lesion assay (PLA) test, as disclosed herein. An email may be generated and sent to the patient to inform the patient that a PLA test has been ordered and include a description of the PLA sampling process, as described herein. An email may include an estimate of how long the PLA sampling and evaluation process will take. The patient may also be provided a cost estimate of conducting the PLA test. In some embodiments, the network connects to an insurance database and utilizes the patient's insurance information to provide the cost estimate of the PLA test. In some embodiments, the network is notified of the request for a PLA test submitted by the healthcare provider.

In some embodiments, the system includes a plurality of testing facilities who have registered to be part of a PLA testing network 1540. Upon requesting a PLA test, a testing facility of the network 1540 may be contacted and requested to handle the testing of the PLA test. A testing facility may be selected based on proximity to the patient. In some embodiments, payment submitted by the patient and/or the patient's insurance provider is transferred to the testing facility via the network.

In some embodiments, the system generates an email to inform the patient that that a PLA test has been ordered. In some embodiments, the email includes a description of the process for performing the test. In some embodiments, the email may include a link to a document (i.e., an electronic document, such as a PDF) describing the test, what it measures, and how the collection process will work. In some embodiments, the link also includes access to a sample collection video providing instruction on how to utilize a PLA testing kit. The email may contain a link, selectable by the user or patient, to confirm that they agree to have the test performed and that they are liable for cost if the cost is not covered by their insurance. Upon agreeing, a PLA testing kit, as described herein, may be sent to the patient.

In some embodiments, the system connects to a healthcare provider to schedule a supervised sample collection via a video teleconference. In some embodiments, the healthcare provider which conducts the supervised sample collection is the same healthcare provider who reviewed the images submitted by the patient. In some embodiments, the healthcare provider inputs their schedule to allow a patient to select an available time for a supervised sample collection. In some embodiments, the confirms the scheduled time for the supervised sample collection. The system may populate a patient and/or healthcare provider calendar with the confirmed time for the supervised sample collection.

In some embodiments, a patient-facing device 1510 is connected directly to a device facing the healthcare provider 1520. In some embodiments, the healthcare provider initiates video call with patient and supervises sample collection process.

The healthcare provider may then confirm that sample collection is complete, via an email to the system. The completed sample kit may then be shipped by the patient to a testing facility. In some embodiments, the testing facility is the same facility of the healthcare provider who supervises the sample collection.

In some embodiments, the testing facility reports the results of the PLA test to the network 1505. If the PLA test results are negative, the network may send the results via email to the prescribing healthcare provider. The results may include a PDF report. In some embodiments, the prescribing healthcare provider reviews the email and clicks a link to “send to patient.” System may automatically generate an email to patient with test results including a PDF report and language about a recommended period of follow up for any concerns.

If the PLA test is positive, the system may send the results of the test to the prescribing healthcare provider. The prescribing healthcare provider may recommend the patient should be seen immediately by a healthcare provider in-person. The healthcare provider may click on a link to “recommend an office visit immediately.” In some embodiments, a list of healthcare providers within proximity of the patient are provided to the prescribing healthcare provider. In some embodiments, the prescribing healthcare provider schedules an appointment with the patient if they are within proximity of the patient's location. In some embodiments, the prescribing healthcare provider selects a healthcare provider within proximity of the patient to refer the patient to for an in-person or follow-up consultation. In some embodiments, an email to the patient is generated with the referral to a local healthcare provider for the patient. The email may include the healthcare provider the patient is being referred to, the reason for the referral, and images of the area of concern.

In some embodiments, the system generates an email to the referred healthcare provider with patient information, images of the area of concern, and the PLA test results. In some embodiments, a follow-up consultation comprises a video call with the patient, allowing the healthcare provider to discuss the PLA test results. The healthcare provider may recommend an in-person appointment for further evaluation or biopsy. In some embodiments, the referred healthcare provider registers with the system, such that payment for follow-up consultations or procedures is transferred from the patient or insurance company to the referred healthcare provider.

In some embodiments, as depicted by FIG. 15, a teledermatology platform described herein includes a back-end (e.g., cloud-based) system 1545 accessible via the network 1505. In further embodiments, the back-end system 1545 includes one or more software programs or modules configured to provide cloud-based operations to facilitate skin and skin condition analysis, such as, by way of examples, a computer vision module 1546, a skin condition evaluation module 1547, a scoring module 1548, and the like. In some embodiments, the output of such module(s) is provided to the healthcare provider at the HCP UI 1525 at, for example, the visit review module 1526 and/or the skin sample results module 1527.

### V. Components of the Skin Collection Kit

Provided herein are methods and systems of skin or tissue sample collection that may be used in combination with the teledermatology system and methods disclosed. Systems and methods may reflect the disclosure of WO/2016/179043, the entire disclosure of which is incorporated herein by reference.

C. Non-Invasive Tape Striping and Analysis

The adhesive skin sample collection kit for use with tape stripping methods is provided as a non-invasive means to collect skin samples with minimal discomfort. Cellular material is isolated from the skin sample and can be utilized in tests that can determine the stage of disease, the risk of disease progression and a patient's likelihood of responding to a particular treatment. Treatments include drug therapies and biopsy. Skin sample cellular materials include nucleic acids, polypeptides, lipids, carbohydrates and small molecules. Nucleic acids include DNA and RNA.

In some embodiments, isolated RNA from a collected skin sample is reverse transcribed into cDNA for amplification by PCR to enrich for target genes. The expression levels of these target genes are quantified by quantitative PCR in a gene expression test. A gene expression test provides information on a gene expression signature associated with a disease. A pigmented lesion assay is an exemplary gene expression test which measures the expression levels of target genes from RNA isolated using the adhesive skin sample collection kit.

For example, in some embodiments, the pigmented lesion assay provides objective information on a gene expression signature associated with melanoma. This information can be used to help support a histopathologic diagnosis or to determine the need for a biopsy, thereby reducing unnecessary biopsy procedures. The development of invasive tumor properties is also controlled by gene expression; therefore, the pigmented lesion assay may also differentiate invasive melanoma from melanoma in situ as well as provide staging information. The identification of invasive melanoma with metastatic potential will direct treatments to only those who need it. Another gene expression assay may determine if a melanoma tumor has spread to the lymph nodes. This test can reduce the need for a sentinel lymph node surgery, which can be extensive, cause morbidity and has significant medical costs.

Gene expression analyses facilitate drug development by identifying drug targets and stratifying patients into groups that will maximize a drug response. In an exemplary embodiment, a skin sample collected from the face of a subject with lupus is isolated and utilized in a gene expression test to assess the expression of target genes indicated in lupus drug effects. This gene expression test can identify responders to therapy and identify new drug targets. The use of the adhesive tape allows for skin sample collection without the scarring that can occur with a biopsy.

In some embodiments, one or more polypeptides isolated from the used adhesive tape are detected and/or quantified. For example, in some embodiments, one or more polypeptides isolated from the used adhesive tape are detected and/or quantified using ELISA, immunohistochemistry, mass spectrometry, and/or absorbance measurement. In some embodiments, the sequence of DNA isolated from the used adhesive tape is determined using gene sequencing methods known to one of skill in the art.

In some instances, the skin sample collected using the tape stripping method is used in combination with other clinical assays including immunohistochemistry, immunophenotyping, fluorescent in situ hybridization (FISH), and/or any combination thereof. The skin sample does not necessarily need to be removed from the adhesive tape to prove useful as an assay component. Cellular material from the skin samples can be detected from the surface of the adhesive tape matrix. Detection methods include the use of probes configured to bind to cellular material adhered to the adhesive tape matrix. Probes include, but are not limited to, primers configured to bind to nucleic acids, and antibodies configured to bind to polypeptides, nucleic acids, small molecules, lipids, and/or carbohydrates.

In some embodiments, the tape stripping method is part of the work up for a variety of suspected skin conditions including, but not limited to, lupus, rubeola, acne, hemangioma, psoriasis, eczema, candidiasis, impetigo, shingles, leprosy and Chron's disease. Skin conditions also include inflammatory dermatoses, bullous diseases, infections and cancers. Skin cancers include, but are not limited to, basal cell carcinoma, actinic keratoses, Merkel cell carcinoma, sebaceous carcinoma, squamous cell carcinoma, melanoma and dermatofibrosarcoma protuberans.

In some embodiments, the tape stripping method is performed using a plurality of adhesive tapes. Between 1 and 8 adhesive tapes can be sequentially applied and removed to collect a skin sample. The number of adhesive tapes used per skin sample may include, but is not limited to, 12, 11, 10, 9, 8, 7, 6, 5, 4, 3, 2, 1, from about 2 to about 7, from about 3 to about 6, and from about 4 to about 5. In certain instances, an adhesive tape is applied to the skin and removed from the skin about 1 to about 8 times.

D. Components of the Collection Kit

The adhesive tape of the adhesive skin sample collection kit typically comprises a first collection area comprising an adhesive matrix and a second area extending from the periphery of the first collection area. The adhesive matrix is located on a skin-facing surface of the first collection area. The second area functions as a tab, suitable for applying and removing the adhesive tape. The tab is sufficient in size so that while applying the adhesive tape to a skin surface, the applicant does not come in contact with the matrix material of the first collection area. In some embodiments, the adhesive tape does not contain a second area tab. In some instances, the adhesive tape is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive tape does not comprise latex, silicone, or both. In some instances, the adhesive tape is manufactured by applying an adhesive material as a liquid-solvent mixture to the first collection area and subsequently removing the solvent.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive tape comprises a flexible material, enabling the tape to conform to the shape of the skin surface upon application. In some instances, at least the first collection area is flexible. In some instances, the tab is plastic. In an illustrative example, the adhesive tape does not contain latex, silicone, or both. In some embodiments, the adhesive tape is made of a transparent material, so that the skin sampling area of the subject is visible after application of the adhesive tape to the skin surface. The transparency ensures that the adhesive tape is applied on the desired area of skin comprising the skin area to be sampled. In some embodiments, the adhesive tape is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive tape of this invention is provided on a peelable release sheet in the adhesive skin sample collection kit. In some embodiments, the adhesive tape provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

The peelable release sheet is configured to hold a plurality of adhesive tapes, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The peelable release sheet is configured to hold about 12 adhesive tapes. The peelable release sheet is configured to hold about 11 adhesive tapes. The peelable release sheet is configured to hold about 10 adhesive tapes. The peelable release sheet is configured to hold about 9 adhesive tapes. The peelable release sheet is configured to hold about 8 adhesive tapes. The peelable release sheet is configured to hold about 7 adhesive tapes. The peelable release sheet is configured to hold about 6 adhesive tapes. The peelable release sheet is configured to hold about 5 adhesive tapes. The peelable release sheet is configured to hold about 4 adhesive tapes. The peelable release sheet is configured to hold about 3 adhesive tapes. The peelable release sheet is configured to hold about 2 adhesive tapes. The peelable release sheet is configured to hold about 1 adhesive tape.

The adhesive tape is applied to the skin and removed from the skin. After removing the used adhesive tape from the skin surface, the tape stripping method further comprises storing the used tape on a placement area sheet, where the tape remains until the skin sample is isolated or otherwise utilized. The used tape is configured to be stored on the placement area sheet for at least 1 week at temperatures between −80° C. and 30° C. In some embodiments, the used tape is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used tape on the placement area sheet, the removable liner is removed. The placement area sheet is configured to hold a plurality of adhesive tapes, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The placement area sheet is configured to hold about 12 adhesive tapes. The placement area sheet is configured to hold about 11 adhesive tapes. The placement area sheet is configured to hold about 10 adhesive tapes. The placement area sheet is configured to hold about 9 adhesive tapes. The placement area sheet is configured to hold about 8 adhesive tapes. The placement area sheet is configured to hold about 7 adhesive tapes. The placement area sheet is configured to hold about 6 adhesive tapes. The placement area sheet is configured to hold about 5 adhesive tapes. The placement area sheet is configured to hold about 4 adhesive tapes. The placement area sheet is configured to hold about 3 adhesive tapes. The placement area sheet is configured to hold about 2 adhesive tapes. The placement area sheet is configured to hold about 1 adhesive tape.

The used tape is stored so that the matrix containing, skin-facing surface of the used tape is in contact with the placement area sheet. In some instances, the placement area sheet is a panel of the tri-fold skin sample collector. In some instances, the tri-fold skin sample collector may further comprise a clear panel. The tri-fold skin sample collector may be labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive tapes stored on a peelable release panel. In some instances, the tri-fold skin sample collector further comprises a placement area panel with a removable liner. The tape stripping method involves removing an adhesive tape from the tri-fold skin sample collector peelable release panel, applying the adhesive tape to a skin sample, removing the used adhesive tape containing a skin sample and placing the used tape on the placement area sheet. In some instances, the placement area panel is a single placement area panel sheet. The identity of the skin sample collected is indexed to the tri-fold skin sample collector or placement area panel sheet by using a barcode or printing patient information on the collector or panel sheet. The indexed tri-fold skin sample collector or placement sheet is sent to a diagnostic lab for processing. The used tape is configured to be stored on the placement panel for at least 1 week at temperatures between −80° C. and 25° C. In some embodiments, the used tape is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments the indexed tri-fold skin sample collector or placement sheet is sent to a diagnostic lab using UPS or FedEx.

In an exemplary embodiment, the tape stripping method further comprises preparing the skin sample prior to application of the adhesive tape. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the tape stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The tape stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit comprises at least one adhesive tape, a sample collector, and an instructions for use sheet. In an exemplary embodiment, the sample collector is a tri-fold skin sample collector comprising a peelable release panel comprising at least one adhesive tape, a placement area panel comprising a removable liner, and a clear panel. The tri-fold skin sample collector may further comprise a barcode and/or an area for transcribing patient information. The adhesive skin sample collection kit is configured to include a plurality of adhesive tapes, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the tape stripping method. The instructions for use sheet preferably include diagrams to illustrate the tape stripping method.

In some instances, the adhesive skin sample collection kit provides all the necessary components for performing the tape stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. The components of the skin sample collection kit may be provided in a cardboard box.

### VI. Cellular Material and Sample Process

In some embodiments of the methods described herein, a skin sample is obtained from the subject by applying an adhesive patch to a skin region of the subject. In some embodiments, the skin sample is obtained using an adhesive patch. In some embodiments, the adhesive patch comprises tape. In some embodiments, the skin sample is not obtained with an adhesive patch. In some instances, the skin sample is obtained using a brush. In some instances, the skin sample is obtained using a swab, for example a cotton swab. In some cases, the skin sample is obtained using a probe. In some cases, the skin sample is obtained using a hook. In some instances, the skin sample is obtained using a medical applicator. In some instances, the skin sample is obtained by scraping a skin surface of the subject. In some cases, the skin sample is obtained through excision. In some instances, the skin sample is biopsied. In some embodiments, the skin sample is a biopsy. In some instances, the skin sample is obtained using one or more needles. For example, the needles may be microneedles. In some instances, the biopsy is a needle biopsy, or a microneedle biopsy. In some instances, the skin sample is obtained invasively. In some instances, the skin sample is obtained non-invasively.

In some embodiments, the skin sample comprises cells of the stratum corneum. In some embodiments, the skin sample consists of cells of the stratum corneum. In some embodiments, the skin sample does not include the basal layer of the skin. In some embodiments, the skin sample comprises or consists of a skin depth of 10 μm, 50 μm, 100 μm, 150 μm, 200 μm, 250 μm, 300 μm, 350 μm, 400 μm, 450 μm, 500 μm, or a range of skin depths defined by any two of the aforementioned skin depths. In some embodiments, the skin sample comprises or consists of a skin depth of 50-100 μm. In some embodiments, the skin sample comprises or consists of a skin depth of 100-200 μm. In some embodiments, the skin sample comprises or consists of a skin depth of 200-300 μm. In some embodiments, the skin sample comprises or consists of a skin depth of 300-400 μm. In some embodiments, the skin sample comprises or consists of a skin depth of 400-500 μm.

In some embodiments, the skin sample is no more than 10 μm thick. In some embodiments, the skin sample is no more than 50 μm thick. In some embodiments, the skin sample is no more than 100 μm thick. In some embodiments, the skin sample is no more than 150 μm thick. In some embodiments, the skin sample is no more than 200 μm thick. In some embodiments, the skin sample is no more than 250 μm thick. In some embodiments, the skin sample is no more than 300 μm thick. In some embodiments, the skin sample is no more than 350 μm thick. In some embodiments, the skin sample is no more than 400 μm thick. In some embodiments, the skin sample is no more than 450 μm thick. In some embodiments, the skin sample is no more than 500 μm thick.

In some embodiments, the skin sample is at least 10 μm thick. In some embodiments, the skin sample is at least 50 μm thick. In some embodiments, the skin sample is at least 100 μm thick. In some embodiments, the skin sample is at least 150 μm thick. In some embodiments, the skin sample is at least 200 μm thick. In some embodiments, the skin sample is at least 250 μm thick. In some embodiments, the skin sample is at least 300 μm thick. In some embodiments, the skin sample is at least 350 μm thick. In some embodiments, the skin sample is at least 400 μm thick. In some embodiments, the skin sample is at least 450 μm thick. In some embodiments, the skin sample is at least 500 μm thick.

In some embodiments, the adhesive patch removes a skin sample from the subject at a depth no greater than 10 μm. In some embodiments, the adhesive patch removes a skin sample from the subject at a depth no greater than 50 μm. In some embodiments, the adhesive patch removes a skin sample from the subject at a depth no greater than 100 μm. In some embodiments, the adhesive patch removes a skin sample from the subject at a depth no greater than 150 μm. In some embodiments, the adhesive patch removes a skin sample from the subject at a depth no greater than 200 μm. In some embodiments, the adhesive patch removes a skin sample from the subject at a depth no greater than 250 μm. In some embodiments, the adhesive patch removes a skin sample from the subject at a depth no greater than 300 μm. In some embodiments, the adhesive patch removes a skin sample from the subject at a depth no greater than 350 μm. In some embodiments, the adhesive patch removes a skin sample from the subject at a depth no greater than 400 μm. In some embodiments, the adhesive patch removes a skin sample from the subject at a depth no greater than 450 μm. In some embodiments, the adhesive patch removes a skin sample from the subject at a depth no greater than 500 μm.

In some embodiments, the adhesive patch removes 1, 2, 3, 4, or 5 layers of stratum corneum from a skin surface of the subject. In some embodiments, the adhesive patch removes a range of layers of stratum corneum from a skin surface of the subject, for example a range defined by any two of the following integers: 1, 2, 3, 4, or 5. In some embodiments, the adhesive patch removes 1-5 layers of stratum corneum from a skin surface of the subject. In some embodiments, the adhesive patch removes 2-3 layers of stratum corneum from a skin surface of the subject. In some embodiments, the adhesive patch removes 2-4 layers of stratum corneum from a skin surface of the subject. In some embodiments, the adhesive patch removes no more than the basal layer of a skin surface from the subject.

Some embodiments include collecting cells from the stratum corneum of a subject, for instance, by using an adhesive tape with an adhesive matrix to adhere the cells from the stratum corneum to the adhesive matrix. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, a skin sample is obtained by applying a plurality of adhesive patches to a skin region of a subject in a manner sufficient to adhere skin sample cells to each of the adhesive patches and removing each of the plurality of adhesive patches from the skin region in a manner sufficient to retain the adhered skin sample cells to each of the adhesive patches. In some embodiments, the skin region comprises a skin lesion.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. In some cases, the skin sample adhered to the adhesive matrix comprises or consists of cells from the stratum corneum of a subject. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. In some instances, the nucleic acid is RNA (e.g., mRNA). An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA include, but not limited to, picogram, nanogram, and microgram quantities. In some embodiments, the RNA includes mRNA. In some embodiments, the RNA includes microRNAs. In some embodiments, the RNA includes mRNA and microRNAs.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA include, but not are limited to, picogram, nanogram, and microgram quantities.

In some instances, the nucleic acid is an RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

In additional embodiments, the adhered skin sample comprises cellular material including nucleic acids such as RNA or DNA, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer.

Referring to FIG. 15, in some embodiments, a back-end (e.g., cloud-based) system accessible via the network 1505 includes a software program or module (see, e.g., FIG. 15, 1548 (scoring module)) configured to score a sample based on, for example, gene expression levels. In some embodiments, the sample score is compared with a reference sample score to determine if there is a statistical significance between the gene expression signature and a disease.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis.

In some instances, cells from the stratum corneum layer are obtained, which comprises keratinocytes. In some instances, cells from the stratum corneum layer comprise T cells or components of T cells. In some cases, melanocytes are not obtained from the skin sample.

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, or more than 10 μm, including increments therein.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher, including increments therein.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 500 nanograms, or higher, including increments therein.

In some cases, methods described herein provide less than less than 10%, less than 8%, less than 5%, less than 2%, less than 1%, or less than 0.5% product yield variations between samples, including increments therein.

In some embodiments, a number of cells is obtained for use in a method described herein. Some embodiments include use of an adhesive patch comprising an adhesive comprising a tackiness that is based on the number of cells to be obtained. Some embodiments include use of a number of adhesive patches based on the number of cells to be obtained. Some embodiments include use of an adhesive patch sized based on the number of cells to be obtained. The size and/or tackiness may be based on the type of skin to be obtained. For example, normal looking skin generally provides less cells and RNA yield than flaky skin. In some embodiments, a skin sample is used comprising skin from a subject's temple, forehead, cheek, or nose. In some embodiments, only one patch is used. In some embodiments, only one patch is used per skin area (e.g., skin area on a subject's temple, forehead, cheek, or nose).

In some cases, methods described herein provide a substantially homogenous population of a nucleic acid product. In some cases, methods described herein provide less than 30%, less than 25%, less than 20%, less than 15%, less than 10%, less than 8%, less than 5%, less than 2%, less than 1%, or less than 0.5% contaminants, including increments therein.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods for detecting an expression level of one or more genes of interest from nucleic acids isolated from a biological sample. In some instances, the expression level is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the expression level is determined using PCR. In some instances, the expression level is determined using qPCR. In some instances, the expression level is determined using a microarray. In some instances, the expression level is determined by sequencing.

Some embodiments include measuring a microRNA. In some embodiments, the measurement includes use of a stem-loop primer. Some embodiments include the use of poly-A tailing. Some embodiments include a pre-amplification of microRNAs.

### VII. Additional Imaging Apparatus

In some embodiments, an imaging apparatus is used in conjunction with any of a variety of mobile devices, such as a mobile device having a digital camera and a light source to produce digital images of an object. The apparatus may include an optical system for illuminating an object to be imaged with uniform light. In some embodiments the apparatus further includes light conduit for delivering light reflected from the object to the mobile device digital camera. In combination with the digital camera of the mobile device, the apparatus may be used to produce digital images that are magnified and/or substantially unobstructed by reflected ambient and/or stray light. The apparatus may include a connector for attaching the apparatus to the mobile device and/or a mobile device case.

In some embodiments, an imaging apparatus includes a contact lens. The contact lens may be used, for example, to modify the optics of a camera viewing a subject's skin and/or to press three-dimensional features of skin to be imaged into one plane by pressing the contact lens against the skin. When a contact lens is being used, a liquid (such as a gel) may optionally be applied to the surface of skin to be imaged. In some embodiments, the contact lens does not modify the camera optics and simply provides a window through which skin or other objects may be imaged. Such a window may provide a clean or sterile surface for contact with a subject's skin and/or prevent a gel or liquid applied to the subject's skin from contaminating the rest of imaging apparatus.

The optical system of the imaging apparatus may include a light guide for collecting light from a mobile device light source and uniformly distributing the light to illuminate an object to be imaged. The optical system may further include a light cap for mixing light emitted by the light guide.

In some embodiments, the optical system includes a first light guide arranged to collect light from the light source and a second light guide arranged to uniformly distribute the light from the first light guide to illuminate an object (e.g., a skin lesion). The optical system may further include a light cap arranged to mix light emitted by the second light guide.

### VIII. Applications

In some embodiments, the systems and methods herein are utilized to conduct telehealth visits. The telehealth visits may include teledermatology visits, specializing in skin based healthcare. The treating physician or medical provider may be a dermatologist. The methods herein may be utilized to evaluate a skin condition by capturing images of a region of skin affected by the skin condition.

Skin conditions to be analyzed may include skin cancers, UV damage, acneiform eruptions, autoinflammatory syndromes, chronic blistering, acneiform eruptions, autoinflammatory syndromes, chronic blistering, conditions of the mucous membranes, conditions of the skin appendages, conditions of the subcutaneous fat, congenital anomalies, connective tissue diseases, abnormalities of dermal fibrous and elastic tissue, dermal and subcutaneous growths, dermatitis, disturbances of pigmentation, drug eruptions, endocrine-related skin conditions, eosinophilic skin conditions, epidermal nevi, neoplasms, cysts, erythemas, genodermatoses, infection-related skin conditions, bacterium-related skin conditions, mycobacterium-related skin conditions, mycosis-related skin conditions, parasitic infestations, stings, bites, virus-related skin conditions, lichenoid eruptions, lymphoid-related skin conditions, melanocytic nevi and neoplasms, lmelanoma, monocyte- and macrophage-related skin conditions, mucinoses, neurocutaneous skin conditions, noninfectious immunodeficiency-related skin conditions, nutrition-related skin conditions, papulosquamous hyperkeratotic, palmoplantar keratodermas, pregnancy-related skin conditions, pruritic skin conditions, psoriasis, reactive neutrophilic, recalcitrant palmoplantar eruptions, skin conditions resulting from errors in metabolism, skin conditions resulting from physical factors, ionizing radiation-induced skin conditions, urticaria, angioedema, and vascular-related skin conditions.

Remote dermatology visits may be enabled by image capture systems as described herein. Image capture systems may be camera systems integrated within a personal electronic device, such as a mobile phone or tablet. Use of integrated camera systems paired with the software applications or modules (see, e.g., FIG. 15, 1516) described herein may allow greater accessibility to remote teledermatology evaluations.

In some embodiments, teledermatology evaluations may be utilized for preliminary screening to determine if a patient requires further analysis and/or testing. In some embodiments, testing is carried out by adhesive stripping systems, as described herein. In some embodiments, genetic information is considered when evaluations. The systems and methods herein may be utilized to diagnose skin conditions, prescribe treatments, and develop therapies. In some embodiments, information from genetic and/or epigenetic testing is considered when diagnosing skin conditions and prescribing treatments.

A. Cancer Screening and Evaluation

In some embodiments, the system and methods disclosed herein are utilized to screen for potential skin cancers. The systems and methods herein may be instrumental in catching skin cancer in the early stages. A streamlined and simple user experience can facilitate routine monitoring and tracking of lesions, abnormalities, or moles. Routine tracking can catch changes or signs of developing skin cancer or high risk lesions and moles. Remote teledermatology can facilitate expert diagnosis is a timely manner.

In some embodiments, adhesive stripping systems or other non-invasive sampling methods can be used in conjunction with remote teledermatology for detecting gene expression levels of a gene of interest which may correspond to a skin cancer. In some embodiments, the skin cancer is a non-melanoma skin cancer (NMSC). In some embodiments, the NMSC is basal cell carcinoma (BCC), or squamous cell carcinoma (SCC). In some embodiments, the skin cancer is melanoma. In some embodiments, methods for diagnosing melanoma and/or solar lentigo in a subject by analyzing nucleic acid molecules obtained from the subject may be combined with the teledermatology methods and systems as disclosed herein. The methods may be carried out at the evaluation and testing steps, as disclosed herein. Methods may reflect the disclosure of WO/2009/140550, the entire disclosure of which is incorporated herein by reference.

B. UV Damage Evaluation

In some embodiments, the need for a UV skin damage assessment may be carried out by the systems and methods described herein. In some embodiments, computer systems may be utilized to assess and/or prescribe treatment for regions of skin damaged by UV exposure. In some embodiments, photography or images are used to demonstrate sun damage, and a need for the subject to have a UV skin damage assessment.

In some embodiments, methods and systems disclosed herein relate to a method of detecting, assessing, measuring, or determining the presence of a skin damage such as UV skin damage based on a presence or expression level of the target gene. Some embodiments relate to a method of identifying a subject with UV skin damage based on a presence or expression level of the target gene. Some embodiments relate to a method of identifying a subject with an amount or extent of UV skin damage based on a presence or expression level of the target gene. Some embodiments include detecting, assessing, measuring, or determining the presence of UV skin damage based on a presence or expression level of the target gene. Some embodiments include the use of multiple target genes. In some embodiments, the target genes described herein are used in any method described herein. In some embodiments, the target genes are used to rule out a skin damage other than UV skin damage. In some embodiments, the UV skin damage is caused by the sun. In some embodiments, the UV skin damage is not caused by the sun. Some embodiments include use of one or more target genes in a method described herein.

In some embodiments, the method includes one or more target genes (e.g., 1 target gene, or multiple target genes). In some embodiments, the one or more target genes include a gene encoding a Vitamin A gene family or family member, a gene encoding a Programmed Cell Death Protein gene family or family member, a gene encoding a Small Proline Rich Protein gene family or family member, a gene encoding an Interleukin 1/2 gene family member, a gene encoding a cystatin gene family or family member, or a combination thereof.

In some embodiments, the one or more target genes include a gene encoding a retinoid response gene. In some embodiments, the retinoid response gene encodes a Vitamin A gene family member. In some embodiments, the one or more target genes include a gene encoding a Vitamin A gene family member. An example of a gene encoding a Vitamin A gene family member includes but is not limited to a gene encoding cellular retinoic acid binding protein 2 (CRABP2).

Disclosed herein, in some embodiments, are methods of determining the presence of UV skin damage in a skin sample. Some embodiments include identifying a subject suspected of having UV skin damage. Some embodiments include isolating nucleic acids from a skin sample obtained from the subject. In some embodiments, the skin sample is obtained by applying an adhesive patch to a skin region of the subject. In some embodiments, the adhesive patch is applied in a manner sufficient to adhere skin sample cells to the adhesive patch. In some embodiments, the skin sample is further obtained by removing the adhesive patch from the skin sample in a manner sufficient to retain the adhered skin sample cells to the adhesive patch. In some embodiments, the skin sample cells comprise cells from the stratum corneum. In some embodiments, the skin sample cells consist of cells from the stratum corneum. Some embodiments include isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise or consist of cells from the stratum corneum. Some embodiments include measuring or detecting an expression level of at least one target gene. In some embodiments, the at least one target gene is known to be upregulated or downregulated in subjects with UV skin damage. Some embodiments include contacting the isolated nucleic acids with a set of probes that recognize the target gene. Some embodiments include detecting binding between the at least one target gene and the set of probes.

C. UV Exposure Scores

Disclosed herein, in some embodiments, the evaluation comprises assessment of a UV exposure score. Referring to FIG. 15, in some embodiments, a back-end (e.g., cloud-based) system accessible via the network 1505 includes a software program or module (see, e.g., FIG. 15, 1548 (scoring module)) configured to score a sample based on, for example, UV exposure levels. In some embodiments, the UV exposure score is used in a method described herein. For example, the UV exposure score may be used in a method of detecting, assessing, measuring, or determining the presence of a skin damage such as UV skin damage. In some embodiments, the UV exposure score incorporates the expression level of one or more target genes described herein. Based on a patient's UV exposure score, they may be treated with, or recommended treatment with a skin treatment described herein. In some embodiments, the UV exposure score is generated with a computer or processor. In some embodiments, the UV exposure score is provided to a medical practitioner. In some embodiments, the UV exposure score is provided to a patient or subject.

In some embodiments, the UV exposure score comprises an integer indicative of UV exposure. In some embodiments, the UV exposure, or the UV exposure score, is indicative of sun damage. In some embodiments, the UV exposure score is indicative of UV skin damage. In some cases, a higher UV exposure score indicates more UV exposure or more UV skin damage than a lower score. In some cases, a lower UV exposure score indicates less UV exposure or less UV skin damage than a higher score. Examples of UV exposure scores include integers from 1 to 10. In some embodiments, the UV exposure score is 1, 2, 3, 4, 5, 6, 7, 8, 9, or 10. In some embodiments, the UV exposure score is in a range defined by any two of: 1, 2, 3, 4, 5, 6, 7, 8, 9, or 10.

The UV exposure score may quantitative (e.g., numeric or alphanumeric), with higher or lower resolution (e.g., 1-10 or high/medium/low), or qualitative (e.g., significant increase/decrease relative to a cohort), or the like. In some embodiments, the UV exposure score is quantitative. In some embodiments, the quantitative UV exposure score is numeric. In some embodiments, the quantitative UV exposure score is alphanumeric. In some embodiments, the quantitative UV exposure score is alphabetic. In some embodiments, the quantitative UV exposure score is a value or a range of values such as 1-10 or A-Z. In some embodiments, the quantitative UV exposure score is relative or general, for example: “low,” “medium,” or “high.” In some embodiments, the quantitative UV exposure score is relative to a control UV exposure score, or relative to a baseline (e.g., pre-exposure) UV exposure score.

In some embodiments, the UV exposure score is qualitative. In some embodiments, the qualitative UV exposure score is numeric. In some embodiments, the qualitative UV exposure score is “yes” or “no.” In some embodiments, the qualitative UV exposure score is “significant” or “insignificant.” In some embodiments, the qualitative UV exposure score is a significant increase or decrease relative to a control such as a cohort. In some embodiments, the qualitative UV exposure score is relative to a control UV exposure score, or relative to a baseline (e.g., pre-exposure) UV exposure score.

In some embodiments, the UV exposure score incorporates the expression level of one or more target genes. In some embodiments, an algorithm evaluates the various expression levels and make assumptions or recommendations. In some embodiments, the algorithm uses gene expression data, and/or patient parameters such as age, gender, skin type, history of sun damage, tanning bed use, smoking, sunburns, and the like.

In some embodiments, the UV exposure score incorporates an assessment of a subject's skin condition. In some embodiments, the skin condition is visually assessed and/or scored. In some embodiments, the UV exposure score is increased based on the subject's skin condition, such as a poor skin condition and/or erythema. In some embodiments, the UV exposure score is decreased based on the subject's skin condition, such as a good skin condition and/or lack of erythema.

D. Psoriasis Evaluation

Methods of detecting the presence of an autoimmune disease based on molecular risk factors may be used in combination with the evaluation and testing steps of the teledermatology system and methods disclosed herein. In some instances, a method of detecting the presence of psoriasis, lupus, or atopic dermatitis based on the molecular risk factors is utilized. In some instances, a method of monitoring the progression of an autoimmune disease, e.g., psoriasis, lupus, or atopic dermatitis, based on the molecular risk factors is utilized. Methods may reflect the disclosure of WO/2019/217478, the entire disclosure of which is incorporated herein by reference.

E. Risk Based on Location

In some embodiments, a user or patient associated with a location having a high average UV index may be prompted to frequently submit images lesions, moles, or skin regions which have been damaged by the sun. The software application disclosed herein may utilize the location features of the device which it is loaded on to determine and/or track a user's geographical location. In some embodiments, as depicted in FIG. 6AA, the software application requests access to a device's location data. If a user travels to a region associated with a high average UV index, the software application may generate a notification for the user to consider conducting a skin evaluation. In some embodiments, the system may generate a notification to the user to take precautions to protect their skin from sun exposure by covering up or applying sunscreen upon determining that the user has traveled to an area known for high UV exposure.

In some embodiments, the system notifies a user when the UV index surpasses a given threshold, at their location, on any given day. In some embodiments, the system sends a notification when the UV index is greater than about 6 to about 11. In some embodiments, the system sends a notification when the UV index is greater than about 6 to about 7, about 6 to about 8, about 6 to about 9, about 6 to about 10, about 6 to about 11, about 7 to about 8, about 7 to about 9, about 7 to about 10, about 7 to about 11, about 8 to about 9, about 8 to about 10, about 8 to about 11, about 9 to about 10, about 9 to about 11, or about 10 to about 11. In some embodiments, the system sends a notification when the UV index is greater than about 6, about 7, about 8, about 9, about 10, or about 11, including increments therein. In some embodiments, the system sends a notification when the UV index is greater than at least about 6, about 7, about 8, about 9, or about 10, including increments therein. In some embodiments, the system sends a notification when the UV index is greater than at most about 7, about 8, about 9, about 10, or about 11, including increments therein. In some embodiments, notification of a high UV index is preemptive and the notification is sent 1, 2, 3, 4, 5, 6, 7, 8, 9, 10, 11, 12, 13, or 14 days ahead of a predicted instance of a high UV index in the region which the user is located.

F. Other Applications

While the systems and methods described herein relate to monitoring and assessing various skin conditions which may be associated with UV exposure, melanoma, and other skin conditions, the systems and methods disclosed herein may be applied to applications outside of dermatology. For example, the methods and systems disclosed herein may be utilized to track bruising, healing, parasitic abnormalities, damage to surfaces, viral lesions, and the like.

In some embodiments, the system and methods herein are applied to veterinary practices. For example, the methods and systems herein may be utilized to evaluate and track skin cancer, dermatitis, mange infestations, flea infestations, ringworm, or other health conditions which may cause an abnormal appearance in the skin of an animal.

### IX. Computing System

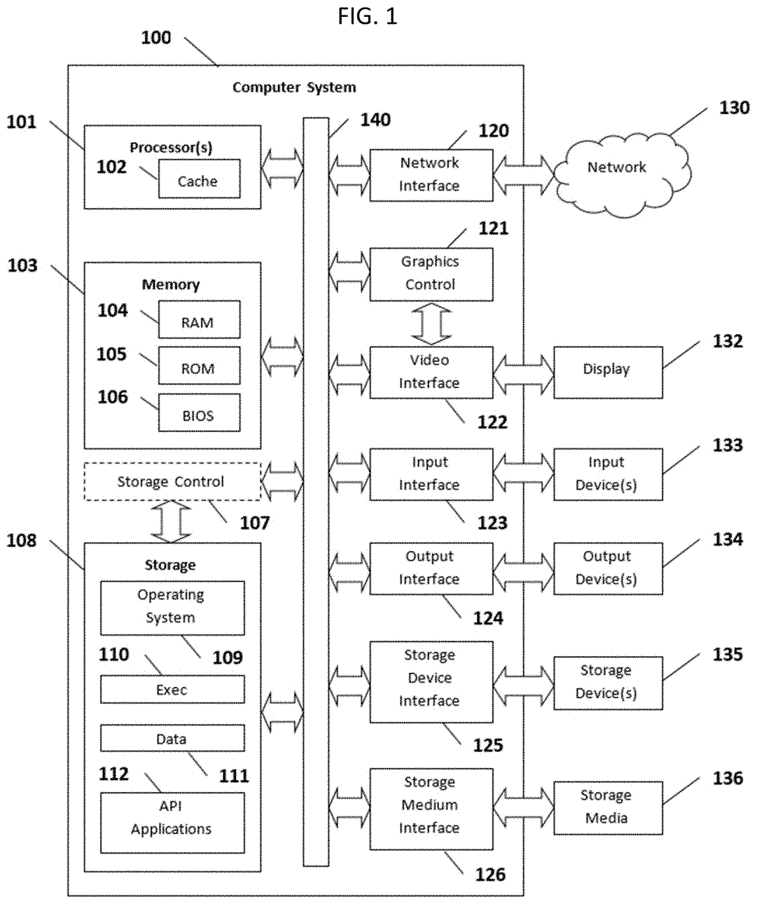

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In some embodiments, the computer system 100 comprises a video or still image capture system. In some embodiments, the image capture system comprises at least two cameras. In some embodiments, the image capture system comprises a single camera system comprising at least two lenses. In some embodiments, the at least two lenses are fixed focal lenses. In some embodiments, the camera system comprises a first lens and a second lens, wherein the first lens and the second lens comprise different focal lengths. A lens of the system may have an optical power of a 2×, 4×, 10×, 25×, 50×, 100×, 1000×, or 2000×, including increments therein. In some embodiments, the lenses of the system allow for simultaneous capture of multiple images. In some embodiments, the lenses comprise different focal lengths and allow for simultaneous capture of images at different zoom levels. In some embodiments, the camera system captures multiple images of a lesion or skin area affected by a skin condition.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition, or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, and personal digital assistants. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetB SD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

Computer systems described herein may be utilized as part of the telemedical systems and methods of the present invention. In some embodiments, a computer system may be utilized as a device configured for use by a patient, partner, caretaker, healthcare provider, or dermatologist (or other physician). In some embodiments, a computer system may be utilized as a device to face a physician, telemedical operator, or other healthcare professional.

### X. Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### XI. Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules or features. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, one or more web applications, one or more mobile applications, one or more standalone applications, one or more web browser plug-ins, extensions, add-ins, or add-ons, or combinations thereof are utilized to perform the telemedical methods as described herein. In some embodiments, one or more web applications, one or more mobile applications, one or more standalone applications, one or more web browser plug-ins, extensions, add-ins, or add-ons, or combinations thereof are utilized as part of the telemedical systems as described herein. In some embodiments, one or more web applications, one or more mobile applications, one or more standalone applications, one or more web browser plug-ins, extensions, add-ins, or add-ons, or combinations thereof are utilized to fully or partially automate the telemedical systems and methods as described herein. In some embodiments, automation allows methods to be carried out which are beyond the limits of what can be processed by a human.

### XII. Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

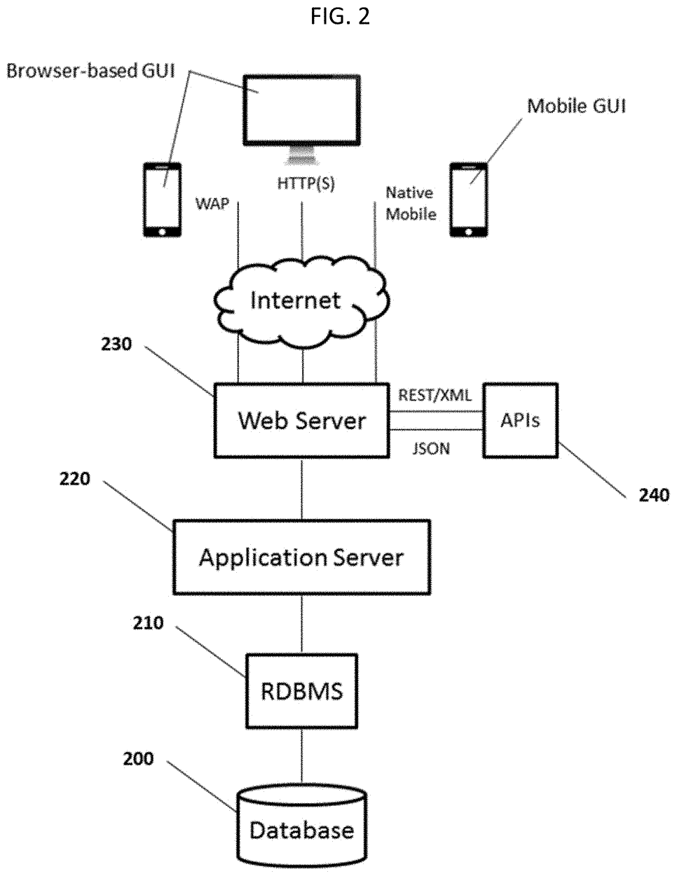

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

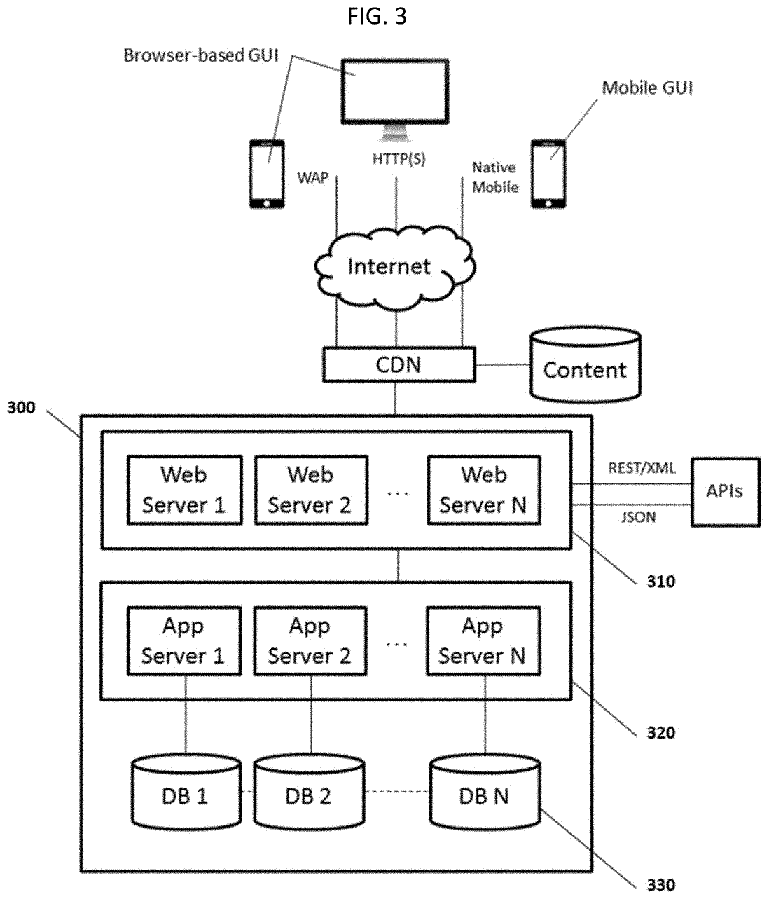

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

The web applications may be utilized as part of the telemedical systems as described herein. The web applications may be utilized to perform the telemedical systems as described herein. In some application, web applications are utilized to provide features or modules of the telemedical systems described herein. In some application, web applications are utilized to fully or partially automate telemedical systems and methods described herein. In some embodiments, automation allows methods to be carried out which are beyond the limits of what can be processed by a human.

### XIII. Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, and Samsung® Apps.

The mobile applications may be utilized as part of the telemedical systems as described herein. The mobile applications may be utilized to perform the telemedical systems as described herein. In some application, mobile applications are utilized to provide features or modules of the telemedical systems described herein. In some application, mobile applications are utilized to fully or partially automate telemedical systems and methods described herein. In some embodiments, automation allows methods to be carried out which are beyond the limits of what can be processed by a human.

### XIV. Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

The standalone applications may be utilized as part of the telemedical systems as described herein. The standalone applications may be utilized to perform the telemedical systems as described herein. In some application, standalone applications are utilized to provide features or modules of the telemedical systems described herein. In some application, standalone applications are utilized to fully or partially automate telemedical systems and methods described herein. In some embodiments, automation allows methods to be carried out which are beyond the limits of what can be processed by a human.

### XV. Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### XVI. Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, software modules are utilized to carry out the telemedical methods as described herein. Software modules may provide features which automate steps of initiating a skin evaluation, evaluating a skin condition, ordering a sample collection kit, reporting results of a sample test, and other steps of the telemedical methods as disclosed herein. In some embodiments, the software modules comprise components of the teledermatology systems as described herein.

### XVII. Databases

In some embodiments, the platforms, systems, media, and methods disclosed herein include one or more databases, or use of the same. In view of the disclosure provided herein, those of skill in the art will recognize that many databases are suitable for storage and retrieval of, for example, patient, photo, video, skin condition, visit, physician, and insurance information. In various embodiments, suitable databases include, by way of non-limiting examples, relational databases, non-relational databases, object oriented databases, object databases, entity-relationship model databases, associative databases, XML, databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### XVIII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

In some embodiments, the systems and methods herein comprise telehealth systems and methods. In some embodiments, the telehealth, telemedicine, or teledermatological applications provided herein comprise the delivery and facilitation of health and health-related services via telecommunications and digital communication technologies. In some embodiments, the telehealth, telemedicine, or teledermatological applications provided herein comprise three main categories including teleconsultation, telementoring, and telemonitoring. In some embodiments of the subject matter described herein, the telehealth, telemedicine, or teledermatological modalities provided herein include store-and-forward, live video, mobile health, remote monitoring, and/or real-time interactive services.

In some embodiments, the telehealth, telemedicine, or teledermatological applications provided herein comprise the exchange of medical information from one location to another using electronic communication. In some embodiments, the telehealth, telemedicine, or teledermatological applications provided herein provide an electronic and/or telecommunications platform and services for use to provide care and/or services at-a-distance.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein and can refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. The term “user” may refer to the patient or subject whose skin is being evaluated in self-administered applications. The term “user” may also refer to a caretaker or individual acting on the patient's behalf. Any steps of the processes to be carried out by a patient-facing or user-facing system, as disclosed herein, may be carried out by the subject whose skin is being evaluated or the individual acting on the patient's behalf.

The term “in vivo” can describe an event that takes place in a subject's body.

The term “ex vivo” can describe an event that takes place outside of a subject's body. An ex vivo assay is not performed on a subject. Rather, it is performed upon a sample separate from a subject. An example of an ex vivo assays performed on a sample is an “in vitro” assay.

The term “in vitro” can describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number can refer to that number plus or minus 10% of that number. The term “about” a range refers to that range minus 10% of its lowest value and plus 10% of its greatest value.

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, the terms “sample” and “biological sample” can refer to any sample suitable for the methods provided by the present invention. A sample of cells can be any sample, including, for example, a skin sample obtained by non-invasive tape stripping or biopsy of a subject, a skin sample obtained by minimally-invasive procedures, or a sample of the subject's bodily fluid. Thus, in one embodiment, the biological sample of the present invention is a tissue sample, e.g., a biopsy specimen such as samples from needle biopsy. In one embodiment, the term “sample” refers to any preparation derived from skin of a subject. For example, a sample of cells obtained using the non-invasive method described herein can be used to isolate nucleic acid molecules or proteins for the methods of the present invention. Samples for the present invention typically are taken from a skin lesion, which is suspected of being the result of a disease or a pathological or physiological state, such as psoriasis or dermatitis, or the surrounding margin or tissue. As used herein, “surrounding margin” or “surrounding tissue” refers to tissue of the subject that is adjacent to the skin lesion, but otherwise appears to be normal or free from lesion. In some embodiments, an “affected area” refers to an area or region of skin affected by a skin condition.

As used herein “corresponding normal cells” or “corresponding normal sample” can refer to cells or a sample from a subject that is from the same organ and of the same type as the cells being examined. In one aspect, the corresponding normal cells comprise a sample of cells obtained from a healthy individual that does not have a skin lesion or skin cancer. Such corresponding normal cells can, but need not be, from an individual that is age-matched and/or of the same gender as the individual providing the cells being examined. Thus, the term “normal sample” or “control sample” refers to any sample taken from a subject of similar species that is considered healthy or otherwise not suffering from the particular disease, pathological or physiological state, or from the same subject in an area free from skin lesions. As such, a normal/standard level of RNA denotes the level of RNA present in a sample from the normal sample. A normal level of RNA can be established by combining skin samples or cell extracts taken from normal healthy subjects and determining the level of one or more RNAs present. In addition, a normal level of RNA also can be determined as an average value taken from a population of subjects that is considered to be healthy, or is at least free of a particular disease, pathological or physiological state. Accordingly, levels of RNA in subject, control, and disease samples can be compared with the standard values. Deviation between standard and subject values establishes the parameters for diagnosing or characterizing disease.

The term “skin” can refer to the outer protective covering of the body, including the epidermis (including the stratum corneum) and the underlying dermis, and is understood to include sweat and sebaceous glands, as well as hair follicle structures. Throughout the present application, the adjective “cutaneous” can be used, and should be understood to refer generally to attributes of the skin, as appropriate to the context in which they are used. The epidermis of the human skin comprises several distinct layers of skin tissue. The deepest layer is the stratum basalis layer, which consists of columnar cells. The overlying layer is the stratum spinosum, which is composed of polyhedral cells. Cells pushed up from the stratum spinosum are flattened and synthesize keratohyalin granules to form the stratum granulosum layer. As these cells move outward, they lose their nuclei, and the keratohyalin granules fuse and mingle with tonofibrils. This forms a clear layer called the stratum lucidum. The cells of the stratum lucidum are closely packed. As the cells move up from the stratum lucidum, they become compressed into many layers of opaque squamae. These cells are all flattened remnants of cells that have become completely filled with keratin and have lost all other internal structure, including nuclei. These squamae constitute the outer layer of the epidermis, the stratum corneum. At the bottom of the stratum corneum, the cells are closely compacted and adhere to each other strongly, but higher in the stratum they become loosely packed, and eventually flake away at the surface.

As used herein, the term “skin lesion” can refer to a change in the color or texture in an area of skin. As such, “skin lesions suspected of being melanoma” can be skin lesions with characteristics of malignant melanoma, which are well known to those of skill in the art, such as dermatologists and oncologists. Such lesions are sometimes raised and can have a color that is different from the color of normal skin of an individual (e.g., brown, black, red, or blue). Lesions suspected of being melanoma sometimes include a mixture of colors, are often asymmetrical, can change in appearance over time, and may bleed. A skin lesion suspected of being melanoma may be a mole or nevus. Melanoma lesions are usually, but not always, larger than 6 mm in diameter. Melanoma includes superficial spreading melanoma, nodular melanoma, acral lentiginous melanoma, and lentigo maligna melanoma. The term “lentigo maligna” refers to a precancerous lesion on the skin, especially in areas exposed to the sun, that is flat, mottled, and brownish with an irregular outline and grows slowly over a period of years. Melanoma can occur on skin that has been overexposed to the sun. Therefore, in one embodiment the skin sample is taken from an area of skin that has been overexposed to the sun.

The term “dysplastic nevus” can refer to an atypical mole or a mole whose appearance is different from that of common moles. Dysplastic nevi are generally larger than ordinary moles and have irregular and indistinct borders. Their color frequently is not uniform and ranges from pink to dark brown; they usually are flat, but parts may be raised above the skin surface. Dysplastic naevus can be found anywhere, but are most common on the trunk of a subject.

The term “cancer” as used herein, can include any malignant tumor including, but not limited to, carcinoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. The term “carcinoma” refers to a malignant new growth made up of epithelial cells tending to infiltrate surrounding tissues, and to give rise to metastases. The term “melanoma” refers to a malignant tumor of melanocytes which are found predominantly in skin but also in bowel and the eye. “Melanocytes” refer to cells located in the bottom layer, the basal lamina, of the skin's epidermis and in the middle layer of the eye. Thus, “melanoma metastasis” refers to the spread of melanoma cells to regional lymph nodes and/or distant organs (e.g., liver, brain, breast, prostate, etc.).

The term “basal cell carcinoma” or “BCC” can refer to a slow-growing neoplasm that is locally invasive but rarely metastasizes. It is derived from basal cells, the deepest layer of epithelial cells of the epidermis or hair follicles. BCC is a common skin cancer that is often associated with overexposure to sunlight.

The term “solar lentigo” or “solar lentigines,” also known as a sun-induced freckle or senile lentigo, can be a dark (hyperpigmented) lesion caused by natural or artificial ultraviolet (UV) light. Solar lentigines may be single or multiple. Solar lentigines are benign, but they do indicate excessive sun exposure, a risk factor for the development of skin cancer. The lesions tend to increase in number with age, making them common among the middle age and older population. They can vary in size from about 0.2 to 2.0 cm. These flat lesions usually have discrete borders, are dark in color, and have an irregular shape.

As used herein, the term “gene” can refer to a linear sequence of nucleotides along a segment of DNA that provides the coded instructions for synthesis of RNA, which, when translated into protein, leads to the expression of hereditary character. As such, the term “skin marker” or “biomarker” refers to a gene whose expression level is different between skin surface samples at the site of malignant melanoma and skin surface samples of normal skin or a lesion, which is benign, such as a benign nevus. Therefore, expression of a melanoma skin marker of the invention is related to, or indicative of, melanoma. Many statistical techniques are known in the art, which can be used to determine whether a statistically significant difference in expression is observed at a high (e.g., 90% or 95%) confidence level. As such, an increase or decrease in expression of these genes is related to and can characterize malignant melanoma. In one embodiment, there is at least a two-fold difference in levels between skin sample near the site of malignant melanoma and skin samples from normal skin.

As used herein, the term “nucleic acid molecule” can mean DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, including increments therein, up to a full-length chromosomal DNA molecule. For methods that analyze expression of a gene, the nucleic acid isolated from a sample is typically RNA.

As used herein, the term “protein” can refer to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

A “probe” or “probe nucleic acid molecule” can refer to a nucleic acid molecule that is at least partially single-stranded, and that is at least partially complementary, or at least partially substantially complementary, to a sequence of interest. A probe can be RNA, DNA, or a combination of both RNA and DNA. It is also within the scope of the present invention to have probe nucleic acid molecules comprising nucleic acids in which the backbone sugar is other that ribose or deoxyribose. Probe nucleic acids can also be peptide nucleic acids. A probe can comprise nucleolytic-activity resistant linkages or detectable labels, and can be operably linked to other moieties, for example a peptide.

A single-stranded nucleic acid molecule is “complementary” to another single-stranded nucleic acid molecule, in certain embodiments of the subject matter described herein, when it can base-pair (hybridize) with all or a portion of the other nucleic acid molecule to form a double helix (double-stranded nucleic acid molecule), based on the ability of guanine (G) to base pair with cytosine (C) and adenine (A) to base pair with thymine (T) or uridine (U). For example, the nucleotide sequence 5′-TATAC-3′ is complementary to the nucleotide sequence 5′-GTATA-3′.

The term “antibody” as used in this invention can include intact molecules of polyclonal or monoclonal antibodies, as well as fragments thereof, such as Fab and F(ab)2, Fv and SCA fragments which are capable of binding an epitopic determinant. The term “specifically binds” or “specifically interacts,” when used in reference to an antibody means that an interaction of the antibody and a particular epitope has a dissociation constant of at least about 1×10-6, generally at least about 1×10-7, usually at least about 1×10-8, and particularly at least about 1×10-9 or 1×10-10 or less.

As used herein “hybridization” can refer to the process by which a nucleic acid strand joins with a complementary strand through base pairing. Hybridization reactions can be sensitive and selective so that a particular sequence of interest can be identified even in samples in which it is present at low concentrations. In an in vitro situation, suitably stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. Hybridization could occur under reduced stringency conditions in about 35% to 25% formamide at about 30° C. to 35° C. In particular, hybridization could occur under high stringency conditions at 42° C. in 50% formamide, 5×SSPE, 0.3% SDS, and 200 mg/ml sheared and denatured salmon sperm DNA. Hybridization could occur under reduced stringency conditions as described above, but in 35% formamide at a reduced temperature of 35° C. The temperature range corresponding to a particular level of stringency can be further narrowed by calculating the purine to pyrimidine ratio of the nucleic acid of interest and adjusting the temperature accordingly. Variations on the above ranges and conditions are well known in the art.

As used herein, the term “mutation” can refer to a change in the genome with respect to the standard wild-type sequence. Mutations can be deletions, insertions, or rearrangements of nucleic acid sequences at a position in the genome, or they can be single base changes at a position in the genome, referred to as “point mutations.” Mutations can be inherited, or they can occur in one or more cells during the lifespan of an individual.

As used herein, the term “kit” or “research kit” can refer to a collection of products that are used to perform a biological research reaction, procedure, or synthesis, such as, for example, a detection, assay, separation, purification, etc., which are typically shipped together, usually within a common packaging, to an end user.

As used herein, the term “ameliorating” or “treating” can mean that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. Thus, a “treatment regimen” refers to any systematic plan or course for treating a disease or cancer in a subject.

The section headings used herein are for organizational purposes only and are not to be construed as limiting the subject matter described.

### XIX. Examples

The following examples are included for illustrative purposes only and are not intended to limit the scope of the invention.

A. Exemplary Method for Providing Evaluation and Sample Collection of a Suspected Skin Lesion for Melanoma

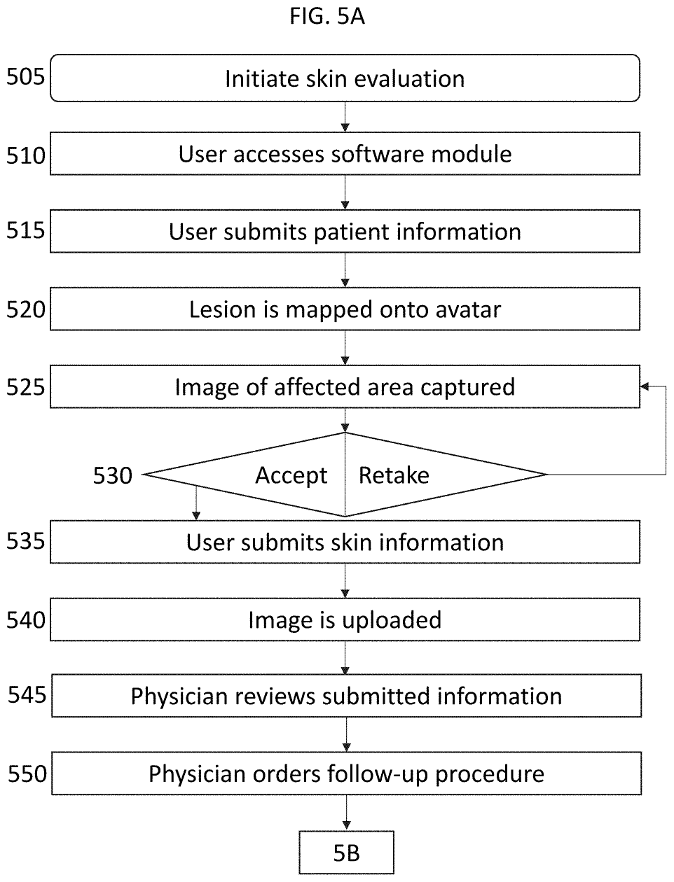

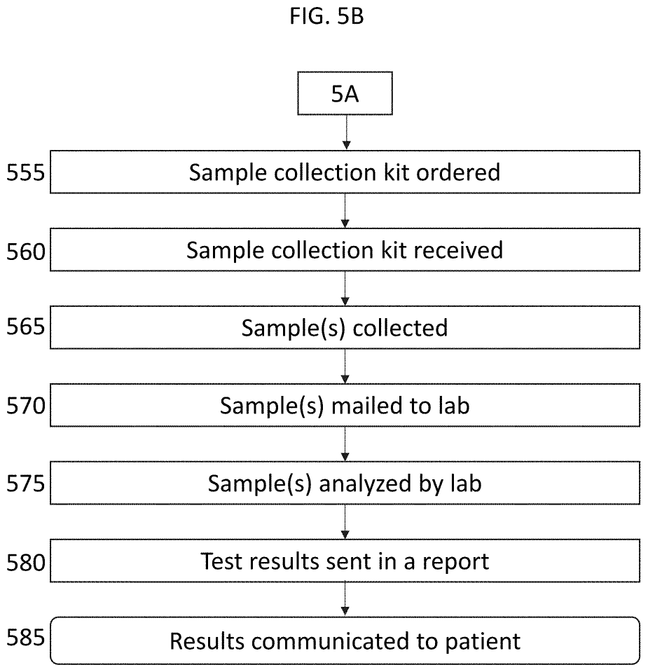

According to some embodiments, an exemplary method of providing evaluation and sample collection of a suspected lesion for melanoma is provided herein. With reference to FIGS. 5A and 5B, an embodiment of an exemplary workflow is depicted.

In some embodiments, at step 505, the skin evaluation is initiated. In some embodiments, to initiate the process, a physician, telemedical, operator, or other profession (simply referred to as a physician herein) will send their patient/s an email with a link to download a mobile application and schedule a virtual visit to capture images mole with the patient. In some embodiments, a caretaker or partner of the patient receives the email and downloads the software application on behalf of a patient.

In some embodiments, a patient, partner, caretaker, or other user will receive an email providing information about the application. The user may then download the software application or be directed to a software module via a mobile or other computing device. At step 510, the user may access the software module or application.

In some embodiments, the user submits patient information at step 515. The user may register or confirm their pre-loaded patient information, such as Name, Age, Mobile Phone, Address, Physician Information, etc. In some embodiments, the user will complete a questionnaire to provide the patient information.

In some embodiments, the application will begin with a greeting and a brief tutorial on how the app works. Upon completing the tutorial, the patient may select a “Got It” or “Continue” button and is taken to the avatar to map a location of a lesion on the body of the patient at step 520.

Again referencing FIG. 5A, once the mole is mapped on the avatar, the user may select “Next” and is taken to the image capture feature. In some embodiments, tips on how to capture a usable image will come on screen for review. The instructions and tips may also be accessed while a in camera mode by pressing a “Help” button. To close the tips window, user may select a “Got It” button or banner.

In some embodiments, the user then points the camera at the affected area, and with one touch on the phone screen the user captures an overview and simultaneously a cropped close-up image of the affected area at step 525. In some embodiments, a programmed feature of the software application extrapolates the cropped up image from raw image captured by the camera.

In some embodiments, when the image is captured, the user will have the ability to frame the close-up image of the mole. In some embodiments, a user will manipulate a yellow frame across the overview image to crop the affected area of the image. In some embodiments, the image of the mole or other skin condition is automatically framed by the application.

In some embodiments, at step 530, the user will have the option to Retake or Accept the captured images. When the images are accepted, the user may be given the option to provide the physician with information about why the mole by selecting the pre-written statements that apply at step 535. In some embodiments, the user will be given the opportunity to Skip the “provide information” step 535. After the user completes the information on the suspicious mole or skips the step, the user may Upload the image and any additional information at step 540.

In some embodiments, when the data uploads, an email notification will be sent to the patient's physician and the images and information will be saved in their patient profile. In some embodiments, the application automatically sends the notification to the physician after the patient uploads the images and corresponding information of one or more areas affected by a skin condition. In some embodiments, the physician opens the email notification and is taken to the portal for review at step 545. In some embodiments, the physician selects the patient name and reviews the images, determining if a home collection kit needs to be sent out or not. In some embodiments, the collection kit is a pigmented lesion assay (PLA) test.

In some embodiments, if a physician determines the affected skin area, lesion, or mole is not a risk, the physician selects “No” and is given the option to email the patient. In some embodiments, an email is automatically drafted and includes details of a risk evaluation or assessment. In some embodiments, the automated email is automatically sent to the patient. In some embodiments, if the physician determines the affected skin area, lesion, or mole may be a risk, the physician selects “Yes.” If the physician selects “Yes′” the physician may be taken to an order form, pre-loaded with the patient information. In some embodiments, the physician then orders a follow-up procedure at step 550. The follow-up procedure may be a request for additional images, a request for an in person consultation, or a request to have a sample collection kit be delivered to a patient.

With reference to FIG. 5B, in some embodiments, a test requisition form is completed and uploaded prior to placing the order to have the sample collection kit sent to the patient. In some embodiments, at step 555, the physician or medical assistant confirms the information, presses Order, and is sent a confirmation and provided the option to send an email to the patient with delivery information, patient-focused instructions for use, a brief video explaining the self-sampling procedure, and clinician contact information.

In some embodiments, at step 560, the patient receives the home collection kit. At step 565, the patient, a partner, or caretaker collects tissue samples from the patient using the home collection kit. In some embodiments, collection of tissue samples is done with the help of the physician (through a teleconference and/or guidance offered within the app). In some embodiments, after collection, at step 570, the patient, partner or caretaker mails or couriers the obtained pigmented lesion sample to a lab to be analyzed. In some embodiments, the sample kit is sent with supervised remote collection.

In some embodiments, the lab assesses the sample, at step 575, and provides the clinician/physician with the test report, at step 580. In some embodiments, the sample test may determine if a suspicious mole is benign or if it has genomic risk factors of melanoma and needs to be biopsied. In some embodiments, the sample test is a DermTech PLA, which is highly validated with proven clinical utility. It has a 91% sensitivity and less than a 1% chance of missing a melanoma (99% NPV).

In some embodiments, at step 585, the physician communicates the test results to the patient within the app/phone/telehealth channels. In some embodiments, communication may be integrated directly with electronic medical accounting (EMA) software for patient billing and filing. Steps of the exemplary method may be automated by various features or modules of a software or web-based application, as disclosed herein.

B. Exemplary Method of a Patient Utilizing Embodiments the Software Applications Disclosed Herein

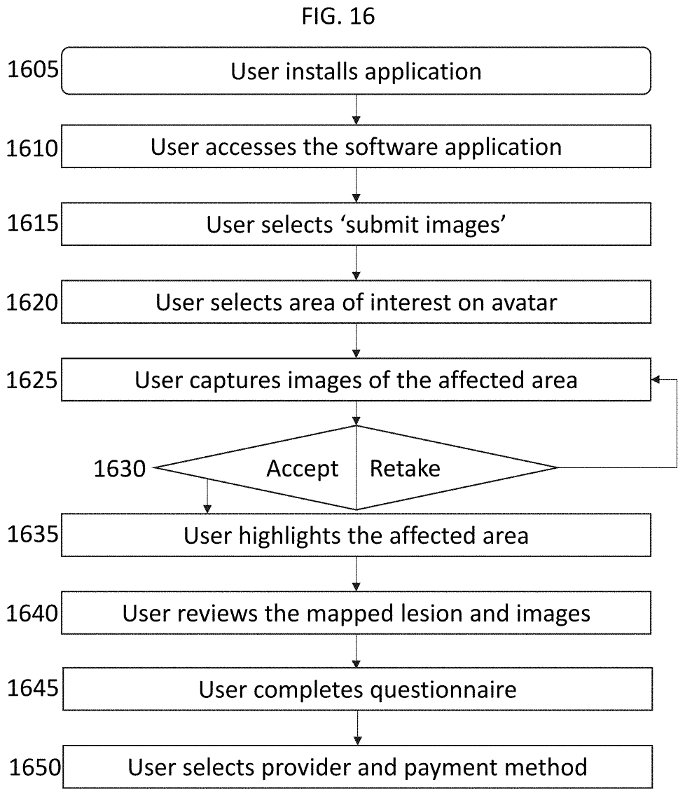

With reference to FIG. 16, an embodiment of an exemplary process for capturing images and submitting data depicted corresponding to an affected region of skin is depicted. In some embodiments, at step 1605, a patient or user downloads the software application for capturing and submitting images of a skin condition to a physician or dermatologist. At step 1610, the user may access the software application by registering credentials. In some embodiments, the user accesses a login user interface 690. The user may select the “register” button 687 to create credentials or select the login button 686 to utilize existing credentials to access the software module or application. In some embodiments, when a user selects the “register” button 687 the registration user interface 690 is loaded onto the device. The user may create custom credentials by selecting the “create your own” custom registration button 692 or use existing credentials from another database by selecting a linked registration button 691.

Once the user gains access to the software application, they are provided with the home screen user interface 601 and provided the option to submit new images by selecting the “submit images” button 602 or review results of an evaluation or lab test by selecting the “check results” button 603. In some embodiments, the software application provides one or more navigation buttons 605 to facilitate navigation of the software application.

When providing images and information for a new skin evaluation, the user may select the “submit images” button 602 at step 1615. In some embodiments, the application then loads the avatar user interface 630. In some embodiments, a gesture information interface or window 629 to present the user with applicable gestures to control the avatar user interface.

In some embodiments, at step 1620, the user manipulates the avatar 632 of the avatar user interface 630 to locate an select the location representing the lesion or area of interest on the avatar.

In some embodiments, the area of interest is mapped on the avatar, the user may the application loads the image capturing user interface 640. In some embodiments, an instruction window 641 with tips on how to capture a usable image will come on screen for review. In some embodiments, instructions and tips may also be accessed while a in camera mode by pressing a “Help” button.

In some embodiments, at step 1625 user captures images of the affected area by pointing the camera at the affected area then pressing and holding down on a touch screen interface of the device. In some embodiments, the device captures an overview and simultaneously a cropped close-up image of the affected area at step. In some embodiments, a programmed feature of the software application extrapolates the cropped up image from raw image captured by the camera.

In some embodiments, the application will load the confirmation user interface 650 and display the captured images. At step 1630, the user will have the option to retake the images by selecting the “retake” button 651 or accept the captured images by selecting the “image not blurry” button 653. If the user selects the “retake” button 651 the image capturing user interface 640 will be reloaded. If the images are accepted, then the user will be able to highlight the lesion within the captured image by taping on the screen, at step 1635 to create an outline or border 654 about the region or area of interest. Once the affected area is outline, the user may select the “next” button 652.

In some embodiments, the software application will then load the image review user interface 660. In some embodiments, at step 1640, the user reviews the captured images and confirms the marked lesion location 662 mapped out on the avatar 632. If the user approves of the images and marked lesion location, the user then selects the “continue with submission” button and the application loads the questionnaire user interface 670.

In some embodiments, the user then answers the provided question by selecting the radial buttons 671 and checkboxes 672 to complete at least the required questions of the questionnaire. When the questionnaire is complete, the user may select the “continue” button 673 to load the provider selection user interface 680.

In some embodiments, at step 1650 the user selects a payment method and eligible provider using the provider selection user interface 680. The user then confirms their selections by interacting with the “submit and pay” button 683. The captured images and provided information are then uploaded and transmitted to the selected healthcare provider.

C. Exemplary Point of Care Skin Sample Collection

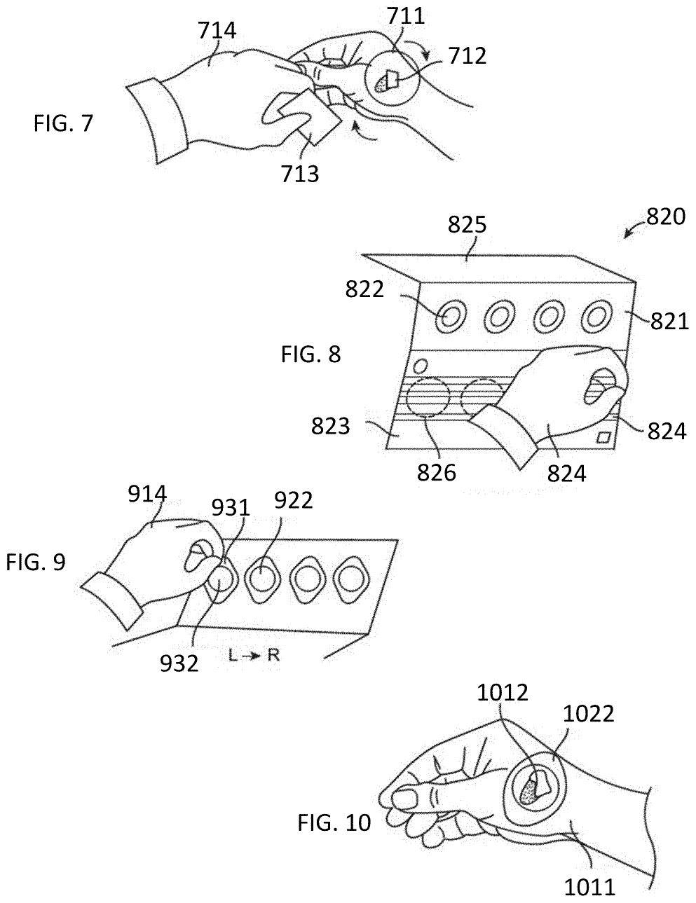

With reference to FIGS. 7-13, an embodiment of a skin sample collection system and method is depicted. In some embodiments, a pigmented lesion located on the hand of a subject is selected for skin sampling. The skin sampling area contains a minimal amount of hair, is not irritated and has not been previously biopsied. The lesion is about 8 mm in size. As exemplified in FIG. 7, the skin sampling area 711 comprising the skin lesion 712 is cleansed with an alcohol pad 713 by a practitioner 714 wearing gloves, and the skin is allowed to air dry for 5 minutes. The practitioner may be a patient of a self-administered sampling kit or a partner, caretaker, physician, or other professional assisting in the sampling of the patient.

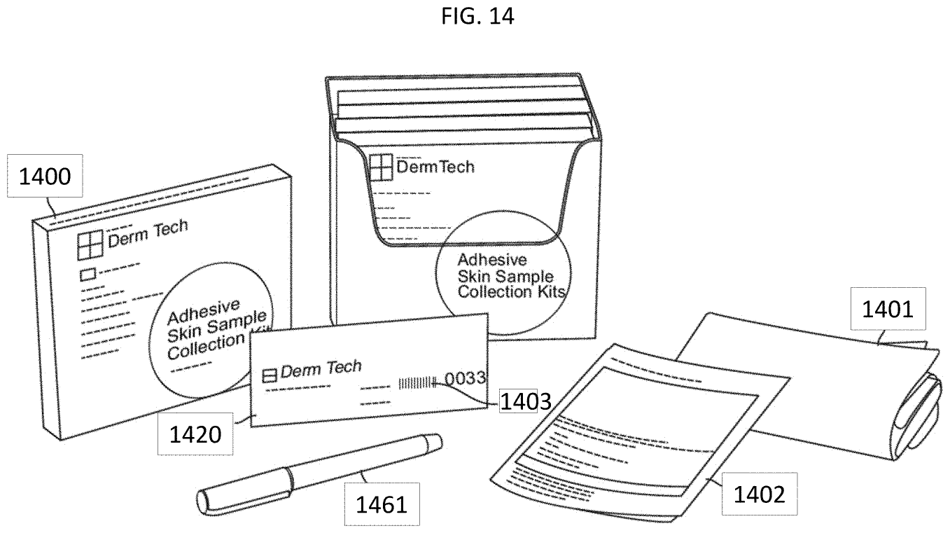

A tri-fold skin sample collector is removed from an adhesive skin sample collection kit exemplified by FIG. 14. FIG. 8 exemplifies the tri-fold skin sample collector 820 comprising a peelable release panel 821 comprising four adhesive tapes 822, a placement area panel 823 comprising a removable liner 824, and a clear panel 825. The tri-fold skin sample collector has a barcode specific for the subject. The removable liner is removed from the placement area panel 823, exposing four regions 826 designated for the placement of up to four used adhesive tapes. The four regions of the placement area panel are not exposed to any skin prior to application of a used tape.

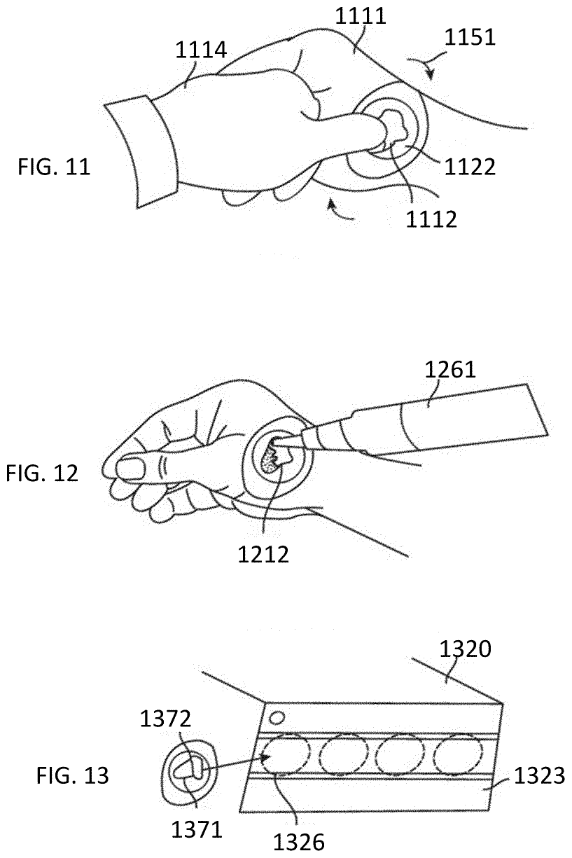

An adhesive tape is removed from the top left side of the peelable release panel as exemplified in FIG. 9. The practitioner 914 handles the adhesive tape 922 by the tab region 931 so that the matrix material of the central collection area 932 does not come in contact with a surface prior to skin application. The skin sampling area is held taut while the adhesive tape is applied onto the skin sampling area. An adhesive tape 1022 positioned on the cleansed skin sampling area 1011 comprising a skin lesion 1012 is exemplified in FIG. 10. The adhesive tape is pressed firmly on the skin while making circular motions. FIG. 11 exemplifies the practitioner 1114 pressing on the skin comprising a skin lesion 1112 while making a circular motion 1151. As exemplified in FIG. 12, the lesion area 1212 is demarcated on the adhesive tape 1222 using a marker 1261 provided in the skin sample collection kit as exemplified by FIG. 14. The practitioner slowly removes the used adhesive tape from the skin sampling area by holding the tab and pulling in one direction. The used tape 1371 comprising a skin sample 1372 is placed on the first unoccupied skin collection region 1326 of the placement area panel 1323 on the tri-fold skin sample collector 1320 as exemplified in FIG. 13. The procedure is repeated with three additional tapes on the same lesion.

The tri-fold skin sample collector is folded and placed in a package provided with the skin sample collection kit. The package contains pre-paid postage and is self-addressed to a processing facility.

D. Exemplary Skin Sample Collection

A pigmented lesion located on the upper back of a subject is selected for skin sampling. The skin sampling area contains a minimal amount of hair, is not irritated and has not been previously biopsied. The lesion is about 15 mm in size. The lesion is sampled utilizing an adhesive skin sample collection kit. The skin sample collection kit includes an instructions for use sheet (or an instruction manual). The lesion is sampled by a capable person who has read and understood the skin sample collection kit instructions for use sheet.

A pair of gloves is removed from the skin sample collection kit and the fitted onto the person performing the skin sampling procedure. The skin sampling area comprising the pigmented lesion is cleansed with an alcohol pad provided in the adhesive skin sample collection kit and the skin is allowed to air dry.

A tri-fold skin sample collector is removed from the adhesive skin sample collection kit. The tri-fold skin sample collector comprises a peelable release panel comprising four adhesive tapes, a placement area panel comprising a removable liner, and a clear panel. The tri-fold skin sample collector has a barcode specific for the subject. The tri-fold skin sample collector further comprises an area configured for providing patient information. The tri-fold skin sample collector is labeled with the subject's name and identifying information. The removable liner is removed from the placement area panel, exposing four regions designated for the placement of up to four used adhesive tapes. The four regions of the placement area panel are not exposed to any skin prior to application of a used tape.

An adhesive tape is removed from the top left side of the peelable release panel. The adhesive tape is handled by the tab region so that the matrix material does not come in contact with a surface prior to skin application. The skin is held taut while the adhesive tape is applied onto the skin sampling area. The adhesive tape is pressed firmly on the skin while making 10 circular motions. The lesion area is demarcated on the adhesive tape using a marker provided in the adhesive skin sample collection kit. The used tape is slowly removed in one direction by pulling the tab away from the skin. The used tape is placed on the first unoccupied skin collection region of the tri-fold skin sample collector. The skin sample procedure is repeated with three additional tapes on the same skin lesion.

The tri-fold skin sample collector comprising 4 used adhesive tapes is folded and placed in the package provided with the adhesive skin sample collection kit. The package contains pre-paid postage and is self-addressed to a diagnostics facility.

E. Exemplary Collection System

The adhesive skin sample collection kit components are stored in a cardboard box (1400) as exemplified in FIG. 14. The kit contains a tri-fold skin sample collector 1420 comprising four adhesive tapes, instructions for use sheet, a marking pen, a pre-paid, self-addressed shipping package 1401, and a shipping label 1402. The tri-fold skin sample collector comprises three panels including a peelable release panel comprising the four adhesive tapes, a placement area panel comprising a removable liner and a clear panel. The tri-fold skin sample collector further comprises a unique barcode 1403 configured to identify a subject. The adhesive tapes stored on the peelable release panel have an expiry date of 2 years from the date of manufacture. The skin sample collection kit is stored between 10° C. and 30° C. The instructions for use sheet (or instruction manual) include all information necessary to enable a person to understand and perform the method. The instructions for use sheet (or instruction manual) include diagrams describing steps of the skin sample collection method.

F. Exemplary Method for Connecting a Patient with a Dermatologist

In some embodiments, a system is provided to connect a patient with a healthcare professional for purposes of evaluating lesions, moles, or other skin anomalies. The healthcare professional may be a general physician, nurse practitioner, or dermatology specialist.

In an embodiment, as depicted by FIG. 15, a user connects to a network 1505 using with a patient-facing device 1510. If the patient has not used the system before, she/he registers by inputting their information. In some embodiments, registration information may comprise the name, address, email, and phone number of the patient. In some embodiments, registration comprises consenting to one or more user agreements, which may include information data sharing agreements, privacy agreements, and the like. In some embodiments, the user consents to allowing the system to access one or more hardware devices of the patient-facing device 1515.

The user may be provided with a list of selectable insurance providers. The user may input the patient's insurance information. In some embodiments, the network 1505 provides a cost of the image review to the user. If insurance will not cover the image review process, the user may provide payment information to the system.

In some embodiments, the network sends a link a mobile phone or device of the patient. The link may be selected by the patient to begin the image collecting process. The user is prompted to capture one or more images of the skin anomaly using a camera of the patient-facing device 1515. In some embodiments, the user is asked if the captured images appear blurry. If the user selects that the images appear blurry, the user may be prompted to retake the images. In some embodiments, the user is asked to highlight the area of concern for the captured images. The user may create a circle around the area of concern using a touchscreen interface of their device.

The user may then upload the images onto the network and inputs information corresponding to each uploaded image of an area concern. The images and corresponding are compiled as a request and sent to an image review queue. The user may receive confirmation that the images have been successfully submitted. In some embodiments, the confirmation is an email confirmation. In some embodiments, the confirmation in includes an approximation of when a response should be received.

In some embodiments, a plurality of health care providers which have opted in or registered to review images uploaded to the network by users of the system are notified of the request uploaded by the patient/user. A healthcare provider may then choose to review the submitted request and the request will be removed from the queue as it has been taken by the healthcare provider.

In some embodiments, the uploaded images and user submitted fields are displayed on a healthcare provider UI 1525 (e.g., a UI configured for use by a healthcare provider, dermatologist or other physician, caretaker, etc.) loaded onto a healthcare provider-facing device 1520, including a visit review module 1526, provided by the network 1505. If the healthcare provider deems the images are too blurry the healthcare provider submits a request for the user to retake images of the area of concern.

If the healthcare provider is able to determine that the area of concern is clearly benign, they may submit their evaluation and communicate that no follow-up is required. The system may generate an email to the patient to confirm that the area of concern is benign.

If the healthcare provider determines that the area of concern is questionable, the healthcare provider may prescribe a pigmented lesion assay (PLA) test, as disclosed herein. An email may be generated and sent to the patient to inform the patient that a PLA test has been ordered and include a description of the PLA sampling process, as described herein.

In some embodiments, the system generates an email to inform the patient that that a PLA test has been ordered. The email may contain a link, selectable by the user or patient, to confirm that they agree to have the test performed and that they are liable for cost if the cost is not covered by their insurance. Upon agreeing, a PLA testing kit, as described herein, may be sent to the patient.

The patient may schedule an appointment through the system for a supervised sample collection with the healthcare provider via a video teleconference. In some embodiments, the healthcare provider inputs their schedule to allow a patient to select an available time for a supervised sample collection. In some embodiments, the confirms the scheduled time for the supervised sample collection. The system may populate a patient and/or healthcare provider calendar with the confirmed time for the supervised sample collection.

In some embodiments, a patient-facing device 1510 is connected directly to a device facing the healthcare provider 1520. In some embodiments, the healthcare provider initiates video call with patient and supervises sample collection process. The completed sample kit may then be shipped by the patient to a testing facility.

In some embodiments, the testing facility reports the results of the PLA test to the network 1505. If the PLA test results are negative, the network may send the results via email to the prescribing healthcare provider. In some embodiments, the prescribing healthcare provider reviews the email and clicks a link to “send to patient.” System may automatically generate an email to patient with test results including a PDF report and language about a recommended period of follow up for any concerns.

If the PLA test is positive, the system may send the results of the test to the prescribing healthcare provider. The healthcare provider may click on a link to “recommend an office visit immediately.” In some embodiments, a list of healthcare providers within proximity of the patient are provided to the prescribing healthcare provider. In some embodiments, the prescribing healthcare provider schedules an appointment with the patient if they are within proximity of the patient's location. In some embodiments, the prescribing healthcare provider selects a healthcare provider within proximity of the patient to refer the patient to for an in-person or follow-up consultation. In some embodiments, an email to the patient is generated with the referral to a local healthcare provider for the patient. The email may include the healthcare provider the patient is being referred to, the reason for the referral, and images of the area of concern.

In some embodiments, the system generates an email to the referred healthcare provider with patient information, images of the area of concern, and the PLA test results. In some embodiments, a follow-up consultation comprises a video call with the patient, allowing the healthcare provider to discuss the PLA test results. The healthcare provider may recommend an in-person appointment for further evaluation or biopsy. In some embodiments, the referred healthcare provider registers with the system, such that payment for follow-up consultations or procedures is transferred from the patient or insurance company to the referred healthcare provider.

The examples and embodiments described herein are for illustrative purposes only and various modifications or changes suggested to persons skilled in the art are to be included within the spirit and purview of this application and scope of the appended claims.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-29. (canceled)

30. A teledermatology system comprising a device comprising at least one processor and instructions executable by the at least one processor to provide a teledermatology application configured to perform operations comprising:
a) receiving a request for teledermatology visit for a skin condition;
b) mapping a location of the skin condition;
c) capturing one or more images of the skin condition;
d) providing information about the skin condition;
e) uploading the location, the one or more images, and the information to a back-end system;
f) receiving a notification of provision of a non-invasive skin tissue sample kit; and
g) receiving results of the skin tissue sample.

31. The system of claim 30, wherein the skin condition comprises melanoma and non-melanoma skin cancer, psoriasis, atopic dermatitis, or sun damage.

32. The system of claim 30, wherein the teledermatology visit is asynchronous.

33. The system of claim 30, wherein the teledermatology application is configured to perform receiving a link from a healthcare provider to initiate the teledermatology visit.

34. The system of claim 30, wherein mapping the location of the skin condition comprises mapping the location onto an avatar.

35. The system of claim 30, wherein capturing the one or more images of the skin condition comprises adjusting illumination, focus, or framing.

36. The system of claim 30, wherein the one or more images comprise a plurality of images and the images are captured substantially simultaneously using more than one camera of the device.

37. The system of claim 30, wherein the one or more images comprise a photo, a video, or a combination thereof.

38. The system of claim 30, wherein providing information about the skin condition comprises responding to a survey by selecting responses from pre-determined options.

39. The system of claim 30, wherein the non-invasive skin tissue sample kit comprises an adhesive sampling material.

40. The system of claim 30, wherein the results of the skin tissue sample comprise one or more genetic or epigenetic risk factors.

41. The system of claim 30, wherein the teledermatology application is configured to perform receiving a request for a follow-up visit.

42. The system of claim 30, wherein the teledermatology application is configured to perform receiving notification of a treatment in response to the results of the skin tissue sample, the teledermatology visit, or a combination thereof.

43. A teledermatology method comprising:
a) receiving a request for teledermatology visit for a skin condition;
b) accessing a mobile teledermatology app via a mobile device;
c) using the mobile teledermatology app to map a location of the skin condition;
d) using the mobile teledermatology app to capture one or more images of the skin condition;
e) using the mobile teledermatology app to provide information about the skin condition;
f) using the mobile teledermatology app to upload the location, the images, and the information to a back-end system;
g) receiving a non-invasive skin tissue sample kit and perform the sampling; and
h) receiving results of the skin tissue sample.

44. The method of claim 43, wherein the skin condition comprises melanoma and non-melanoma skin cancer, psoriasis, atopic dermatitis, or sun damage.

45. The method of claim 43, wherein the teledermatology visit is asynchronous.

46. The method of claim 43, wherein the method further comprises receiving a link from a healthcare provider to access a mobile teledermatology application.

47. The method of claim 43, wherein mapping the location of the skin condition comprises mapping the location onto an avatar.

48. The method of claim 43, wherein capturing the one or more images of the skin condition comprises adjusting illumination, focus, or framing.

49. The method of claim 43, wherein the one or more images comprises a plurality of images and the images are captured substantially simultaneously using more than one camera of the mobile device.

50. The method of claim 43, wherein the one or more images comprise a photo, a video, or a combination thereof.

51. The method of claim 43, wherein providing information about the skin condition comprises responding to a survey by selecting responses from pre-determined options.

52. The method of claim 43, wherein the non-invasive skin tissue sample kit comprises an adhesive sampling material.

53. The method of claim 43, wherein the results comprise one or more genetic or epigenetic risk factors.

54. The method of claim 43, wherein the method further comprises receiving a request for a follow-up visit.

55. The method of claim 43, wherein the method further comprises receiving a treatment in response to the results of the skin tissue sample, the teledermatology visit, or a combination thereof.

56.-85. (canceled)
